(19) United States
(12) Patent Application Publication
(10) Pub. No.: US 2021/0285940 A1
(43) Pub. Date: Sep. 16, 2021

(54) Improved Fluorescent Resonance Energy Transfer-Based Biosensor Proteins And Their Methods of Use Thereof

(71) Applicant: Pebble Labs Inc., Los Alamos, NM (US)

(72) Inventors: Richard Sayre, Los Alamos, NM (US); Mikhail Sinev, Los Alamos, NM (US); Elena Sineva, Los Alamos, NM (US); Tim Travers, Los Alamos, NM (US)

(21) Appl. No.: 17/275,580

(22) PCT Filed: Sep. 12, 2019
(86) PCT No.: PCT/US2019/050813

§ 371 Date: Mar. 11, 2021

Related U.S. Application Data
(60) Provisional application No. 62/730,424, filed on Sep. 12, 2018.

(51) Int. Cl. G01N 33/542; C07K 14/28; C07K 14/435; G01N 21/64

(52) U.S. Cl. CPC G01N 33/542; C07K 14/28; G01N 2021/6432; G01N 21/6428; C07K 2319/60; C07K 14/43595

(57) ABSTRACT

The inventive technology may include a tripartite fluorescent resonance energy transfer (FRET) based fusion protein complex having a ligand binding domain capable of binding to a target ligand and coupled with a donor-acceptor pair of chromophores moieties. The ligand binding domain may be positioned between the donor and acceptor chromophore moieties and may further bind to another ligand binding domain forming a homodimer complex. Ligand binding may cause dissociation of the homodimer complex leading to enhanced donor fluorescence emission and simultaneous quenching of the acceptor fluorescence emission, yielding significant decreases in the acceptor-to-donor fluorescence emission ratio. The tripartite FRET based fusion protein may be used as a biosensor, preferably for molecules responsible for bacterial quorum sensing.

### CROSS-REFERENCE TO RELATED APPLICATIONS

This International PCT application claims the benefit of and priority to U.S. Provisional Patent Application No. 62/730,424, filed Sep. 12, 2018, the disclosure of which is incorporated herein by reference.

### TECHNICAL FIELD

The present invention generally relates to improved fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

### BACKGROUND

Bacteria communicate with each other in a population-dependent manner using a variety of species-specific chemical signal molecules called autoinducers. The process is known as quorum sensing (QS). Autoinducers are synthetized inside bacterial cells, exported into bacterial surroundings, and accumulated there in increasing concentrations. Autoinducers may bind to the protein receptors located in the bacterial membranes. Once the autoinducer concentration exceeds a threshold limit, receptors become saturated with signal molecules. In response to autoinducer binding, their cognate receptors trigger a cascade of regulatory events that change bacterial gene expression patterns, followed by changes in bacterial metabolism and operational mode. QS signal molecules may regulate a diverse array of functions, including antibiotic production, virulence, bio-film formation, stress and defense responses, motility, metabolism, and activities involved in interactions with eukaryotic hosts.

Constitutive expression of pathogenicity genes is costly for bacteria and may lead to decreased adaptation to the environment. Instead, opportunistic bacterial pathogens use multiple signaling systems to manage timely and tightly regulated expression of bacterial toxins and the related virulence factors. In many cases, bacterial pathogenicity traits are regulated by quorum sensing. As generally outlined in FIG. 14, Autoinducer 2, ((AI-2, aka BAI-2 ((2S,4S)-2-methyl-2,3,3,4-tetrahydroxytetrahydrofuran-borate)) is one of the universal chemical signals in the bacterial world. BAI-2 is synthesized by the bacterial cells by LuxS synthase and pumped out into the environment. At sufficiently high concentrations, BAI-2 binds to the receptor LuxP. First found in Vibrio ssp, LuxP and LuxS, protein homologues are widespread in the bacterial kingdom. They regulate expression of pathogenicity factors in many human, animal and food-borne pathogens (See Table 1). Modulation of BAI-2 concentrations is one of the promising approaches to control bacterial microflora without use of antibiotics.

Determination of the concentration of BAI-2 and other signal molecules in environment may predict changes in bacteria collective behavior and forecast potential disease outbreaks. Currently, there is no fast, reliable and quantitative way to measure the concentration of BAI-2 in environmental and industrial samples as well as in the biological liquids. Currently, traditional bioassays for the detection of quorum sensing (QS) molecules, such as BAI-2, take several hours to complete and are subject to substantial environmental and biological perturbations. One attempt to resolve this issue may include the development of more effective biosensors for the detection of QS molecules.

In general, biosensors may operate through catalytic and/or binding principles. In one example, a biosensor for small ligand molecules may include biological constructs that translate a ligand-binding event into a suitable experimental observable event. Fluorescence is one of the most sensitive and, at the same time, relatively simple and convenient ways to detect the binding of biologically important signaling molecules. Fluorescence Resonance Energy Transfer (FRET) is very sensitive to changes in the distances between two fluorescent compounds. Here, one of these compounds, called the Donor (D), is capable of transferring its excitation energy to the other compound, called the Acceptor (A), through a non-radiative resonance mechanism. Thus, FRET may convert ligand binding induced receptor structural transitions into measurable changes in fluorescence. In earlier attempts to apply FRET (or environment sensitive fluorescent probes) to BAI-2 concentration measurements, fluorescence probes were chemically attached to a receptor molecule. One obvious flaw of the above biosensor design is the hydrolytic instability of the probes linkers, which seriously limits their practical implementations.

An alternative to chemical labeling is to use the appropriately matched fluorescent proteins as donor-acceptor FRET pairs fused with the receptor through its N- and C-termini. The great advantage of the fluorescent protein—based biosensors is a long-term chemical stability of the linked polypeptides and the fact they require no post-translational modifications. A FRET-based biosensor employing GFP variants mCFP and mYFP as the donor-acceptor pair fused with the LuxP receptor of Vibrio harveyi demonstrated the applicability of this biosensor design for the quantification of BAI-2 (Rajamani et al., 2007; see also U.S. patent application Ser. No. 11/789,479). However, such FRET-based biosensors are not without their own set of technical difficulties. For example, such constructs must be structurally and functionally optimized to produce a sufficiently clear and diagnostically relevant signal. Notably, U.S. patent application Ser. No. 11/789,479, (by the same listed inventor of the instant application; the specification, biosensor protein and its nucleotide and amino acid sequences and figures are incorporated herein in their entirety by reference), demonstrated a rudimentary FRET-based BAI-2 biosensor employing GFP variants mCFP and mYFP as FRET donor-acceptor pair fused with an intermediate receptor. This system lacked certain technical aspects that significantly limited its functionality and ability to be commercialized.

Indeed, as demonstrated below, the biosensor protein described in the '479 application is limited in its ability to operate as an effective biosensor, in particular of QS molecules. Specifically, the biosensor protein described in the '479 is not practicable to be incorporated into a biosensor device that may allow precise application of the system in a medical or environmental environment. Specifically, this system lacked the ability to generate a diagnostically relevant signal that could be differentiated from the background interference that is present in any FRET or other light-based biosensor detection system. Such deficiencies in the biosensor protein described in the '479 make the results achieved by the current inventive biosensor protein all the more surprising and noteworthy.

Moreover, biosensor protein described in the '479 application was further limited as it was shown to be prone to have a salt induced FRET effect that could mimic binding of a target ligand, such as QS molecules, in environments containing even low amounts of salt thereby decreasing it's reliability and overall utility. Such deficiencies have been overcome in the current invention.

Importantly, the improvements that have been incorporated into the invention's improved biosensor protein were not known, and could not have been considered part of the relevant art at the time of the '479 application. In particular, as will be shown below, the use of the novel Tq and NG as the donor (D) and the acceptor (A) FRET pair to be coupled with LuxP periplasmic receptor (LP) forming one embodiment of the inventive biosensor protein was based on the following enhanced properties of these fluorescent proteins (see FIG. 3 for the Tq and NG spectral data).

These improved features surpass the functionality of the biosensor protein described in the '479 application including, but not limited to: (i) as compared with the previously used mCFP-mYFP donor-acceptor pair in the mCLPY biosensor described in the '479 application, the Tq-NG D-A pair generates a significantly brighter signal (which is due to ˜2.5 fold higher value for the quantum yield of the donor, and 1.3-1.4 fold increase in the acceptor molar extinction and the acceptor quantum yield); (ii) the Tq-NG D-A pair exhibits a larger value for the donor-acceptor Förster distance, i.e., 61 Å vs 51-52 Å for the mCFP-mYFP pair described in the '479 application—providing higher sensitivity for the small changes in transfer efficiency at large D-to-A distances; (iii) Tq may include greater photostability and lower sensitivity to variations in environment conditions (i.e., pH and salt concentration, which as generally used herein may preferably mean NaCl) than CFP allowing its application in a broader range of environmental samples; (iv) Tq and NG moieties has faster folding/maturation time than CFP or YFP which facilitates biosensor production; and (v) finally, the current biosensor protein claimed herein has more than 10-fold higher sensitivity towards apparent dissociation constant of ˜10 nM for BAI-2 binding to TqLPNG biosensor versus 270 nM in the case of the previous mCLPY variant) and adjustable fluorescence response upon autoinducer-2 binding (i.e., up to 48% decrease in the acceptor to donor emission ratio versus 15% decrease in mCLPY). In sum, the biosensor protein configuration claimed herein demonstrates better maturation properties of the Tq- and NG-moieties (as compared with the biosensor protein described in the '479), and demonstrates the improved affinity for BAI-2 binding.

The current technology has overcome the limitations of prior FRET-based biosensors.

Indeed, the current inventive technology includes an improved FRET-based biosensor, TqLPNG, which employs a donor-acceptor FRET pair composed of the newly developed fluorescent proteins with improved properties—monomeric Turquoise2 (Tq, the donor) and monomeric NeonGreen (NG, the acceptor). The advantage of the new TqLPNG biosensor over the previous mCLPY biosensor variant, as noted above specifically includes a higher sensitivity towards BAI-2 concentration, a larger change in fluorescence signal (i.e., acceptor-to-donor emission ratio) for BAI-2 binding, and therefore, smaller background and better accuracy in BAI-2 quantification.

### SUMMARY OF THE INVENTION

The present invention generally relates to improved fluorescent resonance energy transfer protein compounds and methods for using such compounds as biosensors. The present invention also relates to one or more nucleic acids for encoding the protein compounds, vectors containing the nucleic acids, cells transformed by the vectors, and methods for making and using the foregoing compositions.

Another aim of the present invention relates to improved biosensor proteins that may be configured to quantitatively detect QS compounds in a sample or environment. In one preferred embodiment, the present invention relates to improved biosensor proteins for quantitatively detecting the concentration of autoinducer-2 (BAI-2) QS compounds using fluorescent resonant energy transfer (FRET).

One aim of the current invention may include a novel FRET-based biosensor protein which employs an improved donor-acceptor FRET pair. In one preferred embodiment, this improved donor-acceptor FRET pair may be composed of the newly developed fluorescent proteins with improved properties—monomeric Turquoise2 (Tq, the donor) and monomeric NeonGreen (NG, the acceptor).

Yet another aim of the current invention may include a novel FRET-based biosensor protein that may further include a tripartite fluorescent ratiometric sensor protein. In this preferred embodiment, all or part of a ligand binding domain may be fused with a fluorescent protein acceptor and a fluorescent protein donor, such that binding of a ligand to the binding domain may cause a conformational change in the complex and thereby cause a characteristic change in resonance energy transfer. In a preferred embodiment, the biosensor protein may be a fusion protein comprising a LuxP binding moiety, wherein the LuxP moiety is disposed between a donor and acceptor moiety. In this preferred embodiment, the donor moiety may be a fluorescent protein donor moiety connected to the LuxP moiety; and a fluorescent protein acceptor moiety connected to the LuxP moiety, and wherein the donor and acceptor moieties are configured so that they are capable of fluorescent energy transfer when no ligand is bound to the LuxP moiety, which may also be referred generally referred to as a domain, and exhibit diminished fluorescent energy transfer when ligand binds to LuxP. In another improved embodiment, this novel FRET-based biosensor protein may operate in a low salt environment.

For example, in one preferred embodiment, the novel FRET-based biosensor protein of the invention may be used in a sensor device. In this embodiment, the FRET-based biosensor protein may be maintained in a low salt environment, preferably a low NaCl environment. In a preferred embodiment, this low salt or NaCl environment may be between 0.13-0.16 M NaCl, and preferably 0.15 M NaCl.

In another preferred embodiment, the invention may include the improved biosensor protein TqLPNG identified as SEQ ID NO. 2. In this preferred embodiment, improved biosensor protein TqLPNG may include a ligand binding moiety comprising a truncated BAI-2 receptor (LuxP) from Vibrio harveyi, identified as SEQ ID NO. 5, fused to monomeric Turquoise2 (Tq) and monomeric NeonGreen (NG) fluorescent proteins, identified as SEQ ID NOs: 9 and 7 respectively, through the N- and C-terminus of LuxP. In this embodiment, an AI-2 ligand may bind in a concentration dependent manner to the LuxP binding moiety of the TqLPNG biosensor protein. The binding of AI-2 causes alterations in fluorescence resonance energy transfer (FRET) between the fluorescent Tq-moiety (the donor) and the fluorescent NG-moiety (the acceptor). These alterations are attributed to protein structural changes in the LuxP receptor upon BAI-2 binding causing dissociation of the biosensor dimers present, and leading to enhanced Tq-donor fluorescence emission and simultaneous quenching of the NG-acceptor fluorescence emission, yielding significant decreases in the Tq-NG acceptor-to-donor fluorescence emission ratio.

Additional aims of the invention may include isolated nucleic acids coding for the one or more improved biosensor proteins. In one preferred embodiment, the invention may include an isolated nucleic acid coding improved biosensor protein TqLPNG identified as SEQ ID NO: 1.

Another aim of the invention may include protocols for TqLPNGh protein expression and purification. For example, in one embodiment a TqLPNGh protein may be expressed in a genetically modified microorganism, such as a bacterium, yeast or algal cell. Additional embodiment may include generation of a TqLPNGh protein through chemical synthesis.

Another aim of the invention may include expression vectors that express SEQ ID NO: 1. For example, in a preferred embodiment, biosensor protein comprising SEQ ID NO: 1 may be operably linked to a promotor and further part of a plasmid expression vector. In one exemplary embodiment, this plasmid expression vector may include pET-21a(+)-TqLPNGh, identified as SEQ ID NO: 10.

Additional aims of the invention may include a host cell that may further be genetically modified or transformed by one or more expression vectors that express an improved biosensor protein. In one preferred embodiment, a bacteria transformed and expressing the plasmid expression vector pET-21a(+)-TqLPNGh, identified as SEQ ID NO: 10.

Another aim of the invention may include the use of the novel biosensor protein to quantify QS molecules, such as BAI-2 levels in fluid and tissue extracts so as to monitor pathogen population densities as an indicator of the disease state and to better manage disease control strategies in animals, and in particular, humans.

Additional aims of the invention may include the improved biosensor protein TqLPNG identified as SEQ ID NO. 2 to determine the presence of BAI-2 molecules present in environmental and industrial samples as well as in the biological liquids. Additional aims of the invention may include the improved biosensor protein TqLPNG identified as SEQ ID NO. 2 to determine the concentration of BAI-2 molecules present in environmental and industrial samples as well as in the biological liquids. Additional aims of the invention may include the improved biosensor protein TqLPNG identified as SEQ ID NO. 2 in a low salt-environment.

Another aim of the invention may include the use of the novel biosensor protein to quantify BAI-2 levels in fluid and tissue extracts so as to monitor pathogen population densities as an indicator of the disease state and to better manage disease control strategies in aquaculture, and in particular shrimp populations grown in aquaculture.

Additional aspects of the invention may include one or more of the following preferred embodiments:

1. A biosensor for the detection of quorum sensing molecules comprising:
a LuxP binding domain disposed between a Tq protein donor moiety and a NG protein acceptor moiety forming a fusion biosensor protein wherein said LuxP binding domain binds to a LuxP binding domain of a second fusion biosensor protein forming a homodimer fusion biosensor protein;
wherein binding of a quorum sensing molecule to said LuxP binding domain causes dissociation of said homodimer fusion biosensor protein causing enhanced Tq-donor fluorescence emission and simultaneous quenching of NG-acceptor fluorescence emission.
   2. The biosensor protein of embodiment 1, wherein said fusion biosensor protein comprises a fusion biosensor protein selected from the group consisting of: amino acid SEQ ID NOs. 2-3.
   3. The biosensor protein of embodiment 1, wherein said wherein said LuxP binding domain comprises amino acid SEQ ID NO. 5.
   4. The biosensor protein of embodiment 3, wherein said wherein said Tq protein donor moiety comprises amino acid SEQ ID NO. 9.
   5. The biosensor protein of embodiment 3, wherein said, wherein said NG protein acceptor moiety comprises amino acid SEQ ID NO. 7.
   6. The biosensor protein of embodiment 4, wherein said Tq protein donor moiety is connected to the N-terminus of said LuxP binding domain.
   7. The biosensor protein of embodiment 5, wherein said NG protein acceptor moiety is connected to the C-terminus of said LuxP binding domain.
   8. The biosensor protein of embodiment 1, wherein said quorum sensing molecule comprises BAI-2.
   9. The biosensor protein of embodiment 1, wherein said homodimer fusion biosensor protein comprises a homodimer fusion biosensor protein formed in a low salt environment.
   10. The biosensor protein of embodiment 1, wherein said enhanced Tq-donor fluorescence emission and simultaneous quenching of NG-acceptor fluorescence emission comprises a decrease in the Tq-NG acceptor-to-donor fluorescence emission ratio.
   11. The biosensor protein of embodiments 1-6, wherein said fusion biosensor protein comprises a fusion biosensor protein used for one or more of the following: detect aquaculture pathogens; quantify aquaculture pathogens; detect aquaculture pathogen density; quantify aquaculture pathogen density; detect aquaculture pathogen population; quantify aquaculture pathogen population; detect aquaculture pathogen biofilm formation; quantify aquaculture pathogen biofilm formation; detect aquaculture pathogen pathogenicity; and quantify aquaculture pathogen pathogenicity.
   12. The biosensor protein of embodiments 1-6, wherein said fusion biosensor protein comprises a fusion biosensor protein used for one or more of the following: detect BAI-2 in an environmental sample; quantify BAI-2 levels in an environmental sample; detect BAI-2 in a cell sample; quantify BAI-2 levels in a cell sample; detect BAI-2 in a solution sample; quantify BAI-2 levels in a solution sample; detect BAI-2 in a water sample; quantify BAI-2 levels in a water sample; detect BAI-2 in a tissue sample; quantify BAI-2 levels in a tissue sample; detect BAI-2 in an aquaculture sample; quantify BAI-2 levels in an aquaculture sample; detect BAI-2 in a shrimp sample; and quantify BAI-2 levels in a shrimp sample.
   13. The biosensor protein of embodiments 1-6, wherein said fusion biosensor protein is expressed in a prokaryotic cell.
   14. The biosensor protein of embodiments 1-6, wherein said fusion biosensor protein is expressed in a eukaryotic cell.
   15. A tripartite fluorescent ratiometric sensor protein comprising:
a ligand binding domain disposed between a protein donor moiety and a protein acceptor moiety forming a fusion biosensor protein wherein said ligand binding domain binds to a ligand binding domain of a second fusion biosensor protein forming a homodimer fusion biosensor protein;
wherein binding of a ligand to said ligand binding domain causes dissociation of said homodimer fusion biosensor protein causing enhanced donor fluorescence emission and simultaneous quenching of acceptor fluorescence emission.
   16. The tripartite fluorescent ratiometric sensor protein of embodiment 15, wherein said ligand binding domain comprises a LuxP binding domain.
   17. The tripartite fluorescent ratiometric sensor protein of embodiment 16, wherein said LuxP binding domain comprises a truncated BAI-2 receptor (LuxP) from Vibrio harveyi.
   18. The tripartite fluorescent ratiometric sensor protein of embodiment 17, wherein said truncated BAI-2 receptor (LuxP) from Vibrio harveyi comprises amino acid SEQ ID NO. 5.
   19. The tripartite fluorescent ratiometric sensor protein of embodiment 15, wherein said donor protein moiety comprises a mTurquoise2 (Tq) monomeric subunit from Aequorea victoria fused to the N-terminus of said ligand binding domain.
   20. The tripartite fluorescent ratiometric sensor protein of embodiment 19, wherein said mTurquoise2 (Tq) monomeric subunit from Aequorea victoria comprises amino acid SEQ ID NO. 9.
   21. The tripartite fluorescent ratiometric sensor protein of embodiment 15, wherein said acceptor protein moiety comprises a mNeonGreen (NG) monomeric subunit from Aequorea victoria fused to the C-terminus of said ligand binding domain.
   22. The tripartite fluorescent ratiometric sensor protein of embodiment 21, wherein said mNeonGreen (NG) monomeric subunit from Aequorea victoria comprises amino acid SEQ ID NO. 7.
   23. The tripartite fluorescent ratiometric sensor protein of embodiment 15, wherein said fusion biosensor protein comprises a fusion biosensor protein selected from the group consisting of: amino acid SEQ ID NOs. 2-3.
   24. The tripartite fluorescent ratiometric sensor protein of embodiment 15, wherein said ligand comprises a quorum sensing molecule.
   25. The tripartite fluorescent ratiometric sensor protein of embodiment 24, wherein said quorum sensing molecule comprises BAI-2.
   26. The tripartite fluorescent ratiometric sensor protein of embodiment 15, wherein said homodimer fusion biosensor protein comprises a homodimer fusion biosensor protein formed in a low salt environment, which may preferably be 0.15 M NaCl.
   27. The tripartite fluorescent ratiometric sensor protein of embodiment 15, wherein said enhanced Tq-donor fluorescence emission and simultaneous quenching of NG-acceptor fluorescence emission comprises a decrease in the Tq-NG acceptor-to-donor fluorescence emission ratio.
   28. The tripartite fluorescent ratiometric sensor protein of embodiment 15 wherein said fusion biosensor protein comprises a fusion biosensor protein used for one or more of the following: detect aquaculture pathogens; quantify aquaculture pathogens; detect aquaculture pathogen density; quantify aquaculture pathogen density; detect aquaculture pathogen population; quantify aquaculture pathogen population; detect aquaculture pathogen biofilm formation; quantify aquaculture pathogen biofilm formation; detect aquaculture pathogen pathogenicity; quantify aquaculture pathogen pathogenicity; detect BAI-2 in an environmental sample; quantify BAI-2 levels in an environmental sample; detect BAI-2 in a cell sample; quantify BAI-2 levels in a cell sample; detect BAI-2 in a solution sample; quantify BAI-2 levels in a solution sample; detect BAI-2 in a water sample; quantify BAI-2 levels in a water sample; detect BAI-2 in a tissue sample; quantify BAI-2 levels in a tissue sample; detect BAI-2 in an aquaculture sample; quantify BAI-2 levels in an aquaculture sample; detect BAI-2 in a shrimp sample; and quantify BAI-2 levels in a shrimp sample.
   29. The tripartite fluorescent ratiometric sensor protein of embodiment 15, wherein said fusion biosensor protein comprises a fusion biosensor protein used for one or more of the following:
   30. The tripartite fluorescent ratiometric sensor protein of embodiment 15, wherein said fusion biosensor protein is expressed in a prokaryotic cell.
   31. The tripartite fluorescent ratiometric sensor protein of embodiment 15, wherein said fusion biosensor protein is expressed in a eukaryotic cell.
   32. A tripartite fluorescent ratiometric sensor protein comprising amino acid SEQ ID NO. 2, or a sequence having 90% sequence identity with amino acid SEQ ID NO. 2.
   33. A tripartite fluorescent ratiometric sensor protein comprising amino acid SEQ ID NO. 3, or a sequence having 90% sequence identity with amino acid SEQ ID NO. 3.
   34. An isolated a nucleic acid molecule encoding a tripartite fluorescent ratiometric sensor protein comprising SEQ ID NO. 1, or a sequence having 90% sequence identity with nucleotide SEQ ID NO. 1.
   35. An expression vector comprising a nucleic acid molecule encoding tripartite fluorescent ratiometric sensor protein comprising nucleotide SEQ ID NO. 1, or a sequence having 90% sequence identity with nucleotide SEQ ID NO. 1, operably linked to a promoter.
   36. An isolated a nucleic acid molecule encoding tripartite fluorescent ratiometric sensor protein comprising nucleotide SEQ ID NOs. 10 or 11, or a sequence having 90% sequence identity with nucleotide SEQ ID NOs. 10 or 11.

One aim of the invention may include the use of the biosensor, as generally described herein in an opto-electronic hardware device. In one preferred embodiment, such a device may be configured to detect QS molecules.

Additional aims of the inventive technology will be evident from the detailed description and figures presented below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: Structural response of the LuxP (LP) periplasmic receptor of Vibrio harveyi towards binding of the quorum sensing signaling molecule BAI-2 [(2S,4S)-2-methyl-2,3,3,4-tetrahydroxytetra-hydrofuran borate]. The structures of the ligand-free (24-385) LuxP (light gray-colored backbone) and the (24-385) LuxP•BAI-2 complex (black-colored backbone) (PDB entries 1ZHH (Neiditch et al., 2005) and 1JX6 (Chen et al., 2002), respectively) were superimposed using Chimera software (UCSF) (Pettersen et al., 2004).

FIG. 2: Design of the FRET-based TqLPNG biosensor for BAI-2. (2A) Model of TqLPNG biosensor employing mTurquoise2-mNeonGreen fluorescent proteins as the Donor-Acceptor FRET pair to translate the ligand-induced structural changes of LuxP receptor into fluorescence observables. The crystal structures of mTurquoise2 (3ZTF, (Goedhart et al., 2012)) and mNeonGreen (5LTR, (Clavel et al., 2016)) fluorescent proteins were assembled in Chimera to prepare the figure; (2B) Schematic drawing of the TqLPNG biosensor primary structure showing amino acids sequence of linker inserts.

FIG. 3: Spectroscopic properties of the Donor and the Acceptor moieties of the TqLPNGh biosensor. (3A) Absorbance (dotted lines) and fluorescence (solid lines) spectra of mTurquoise2 (the Donor) and mNeonGreen (the Acceptor) fluorescent proteins are shown in black and gray lines, respectively. Absorbance spectra are expressed in units of molar extinctions (M⁻¹ cm⁻¹). Emission spectra are normalized. (3B) Basic characteristics of mTurquoise2-mNeonGreen donor-acceptor FRET pair as compared to those of mCFP-mYFP donor-acceptor pair (used in the earlier biosensor variant by Rajamani et al., 2007).

FIG. 4: Maps of plasmids used in this work. Plasmid design is described in Materials and Methods section. Sequences of the genes and corresponding proteins used for construction of biosensor module are listed below under the section entitled SEQUENCE LISTING. (4A) pET21a(+)—general cloning vector; (4B) pET21-Tqh—plasmid used for expression of mTurquoise2 fluorescent protein (donor); (4C) pET21-NGh—plasmid used for expression of mNeonGreen fluorescent protein; (4D) pET21-TqLPNGh—plasmid used for expression of mTurquoise2-TAAG-(24-365)LuxP-GGAAA-mNeonGreen fusion—biosensor protein

FIG. 5: SDS-PAGE analysis of the TqLPNGh protein expression in E. coli BL21 (DE 3) (luxS⁻) transformed with pET21-TqLPNGh plasmid (A and B), and TqLPNGh purification through affinity chromatography on Talon resin (C and D). (5A) Equal amounts of cells (as based on OD600) were loaded into the lanes 1 and 2, containing the cell culture samples prior and following IPTG induction (at 21° C. for 20 hrs), respectively. (5B) To get the estimate for the total amount of the expressed TqLPNGh, the sample representing the cells (collected following the IPTG induction) from the known volume of the cell culture (1 μL, lane 1) and the sample with the known amount of the purified TqLPNGh (0.25 μG, lane 2) were loaded onto adjacent gel slots. (5C) Essential fractions in the course of the initial TqLPNGh purification. Lanes: 1. 0.5 μL total suspension of the disrupted cells; 2. 1.0 μL soluble fraction (from the suspension of the disrupted cells); 3. 1.0 μL flow-through fraction (i.e., unbound protein species) upon loading of the soluble fraction onto the 5 mL Talon Metal Affinity resin column; 4. 2 μL wash with the binding buffer containing 10 mM imidazole; 5. ˜2 μG TqLPNG from fraction 1 (of 0.45 mL total volume) of the 200 mM imidazole eluate (from the Talon column); 6. ˜2 μG TqLPNG from fraction 2 (of 3.1 mL volume) of the 200 mM imidazole eluate; 7. ˜2 μG TqLPNG from fraction 3 (of 2.1 mL volume) of the 200 mM imidazole eluate; 8. ˜2 μG TqLPNG from fraction 4 (of 1.6 mL volume) of the 200 mM imidazole eluate; 9. ˜1 μG TqLPNG from fraction 4 (of 1.6 mL volume) of the 200 mM imidazole eluate. Concentration of TqLPNGh within the eluted fractions were estimated from their absorption spectra (see panel D of the figure) using ε₅₀₅=116,000 M⁻¹ cm⁻¹ as the value of the protein molar extinction. Molecular weights of the protein ladder (L slots on both panels) are indicated in kDa. (5D) Absorption spectra of fractions eluted from the 5 mL Talon column with 200 mM imidazole containing buffer.

FIG. 6: Purification of TqLPNGh through hydrophobic interection chromatography (HIC) on Butyl Agarose. (6A) About 7 mG TqLPNG (corresponding to ⅓ from the protein collected following its purification on Talon Metal Affinity resin) in 50 mM Tris-HCl (pH 7.5)/0.7 M ammonium sulfate/0.1 mM EDTA/0.5 mM DTT buffer was loaded onto 5 mL HiTrap HP Butyl column equlibrated with the same buffer (i.e., the binding buffer). Following the column wash with ˜25 mL binding buffer, the bound protein species were elluted with a linear gradient of the sulfate-free buffer containing 0.3 M NaCl (the B buffer) using 1 mL/min flow rate. The eluate was fractionated in 2.5 mL portions. (6B) SDS-PAGE analysis of the fractions from the HIC. Lanes: 1. 2 μL of the loaded protein solution; 2. 2 μG TqLPNGh from the fraction 20; 3. 2 μG TqLPNGh from the fraction 21; 4. 2 μG TqLPNGh from the fraction 22; 5. 2 μG TqLPNGh from the fraction 23; 6. 1 μG TqLPNGh from the fraction 24; 7. 0.5 μG TqLPNGh from the fraction 25; 8. 1 μG TqLPNGh from the fraction 19; 9. 2 μG TqLPNGh from the fraction 19. Concentration of TqLPNGh within the eluted fractions were estimated from their absorption spectra. Molecular weights of the protein ladder (L slots on both panels) are indicated in kDa. (6C) Elution profile of the finally purified TqLPNGh preparation in size-exclusion chromatography (SEC) on Superdex 20010/300 column equilibrated with 50 mM HEPES (pH 7.5)/0.1 mM EDTA/0.3 M NaCl (HS300e). Elution profiles for LPNGh (66.3 kDa), NGh (27.5 kDa) and Tqh (27.7 kDa) are shown for comparison. All SEC chromatographies were performed as separate runs under the same conditions in HS300e buffer (50 mM Hepes, pH 7.5, 300 mM NaCl)), 4° C. and 0.5 mL/min flow rate) by using 0.5 mL of 4.5-10 μM protein solution (in HS300e) for injection.

FIG. 7: Spectroscopic properties of the purified TqLPNGh biosensor. Absorbance (ABS) and fluorescence emission (EM) spectra of TqLPNGh are shown as solid and dotted lines, respectively. The spectra refer to 50 mM HEPES (pH 7.5)/0.3 M NaCl/0.1 mM EDTA buffer.

FIG. 8 (A-B): Response of the fluorescence emission spuctrum of TqLPNGh towards BAI-2 binding at different concentrations.of sodium chloride. Emission spectra (solid lines) were deconvoluted into the donor (dotted lines) and the acceptor (dashed lines) emission components, and the evaluation for the FRET efficiency (E values) was performed through enhancement in the acceptor emission referenced to the directly excited acceptor emission (the grey lines) as described under Materials and Methods. The spectra were recorded by using an excitation wavelength of 440 nm. Measurements were performed in a 50 mM HEPES (pH 7.5)/0.1 mM EDTA buffer (containing the specified amount of NaCl) with 50 nM TLPNGh.

FIG. 9: An example of fluorescence emission spectrum of TqLPNGh and its response to BAI-2 binding. (9A) Emisson spectra of 50 nM TqLPNGh in 50 mM HEPES (pH 7.5)/0.1 mM EDTA buffer in the absence (the grey line) and in the presence of 10 μM BAI-2 (the black line). The spectra were recorded by using a 440 nm excitation wavelength. (9B) Dependence of the BAI-2 binding TqLPNGh emission response (expressed as decrease in acceptor-to-donor emission ratio induced by BAI-2 binding) on concentration of NaCl.

FIG. 10: Effect of salt concentration on TqLPNGh dimer formation. (10A) Fluorescence emission spectra of 50 nM ligand-free TqLPNGh in 50 mM HEPES (pH 7.5)/0.1 mM EDTA buffer at different concentration of NaCl: 7.5 (the grey line), 154 (the dotted line), 300 (the dashed line) and 594 mM (the black line) NaCl. The spectra were recorded by using a 440 nm excitation wavelength. (10B) Salt-induced emission response of TqLPNGh: acceptor-to-donor emission ratio and FRET efficiency in the ligand-free TqLPNGh at different concentrations of NaCl. (10C) Association of TqLPNGh monomers into dimers induced by the salt removal. Elution profiles of TqLPNGh in size-exclusion chromatography (SEC) on Superdex 20010/300 column in 50 mM HEPES (pH 7.5)/0.1 mM EDTA buffer containing 0.3 M NaCl (the black solid line) and in the salt-free buffer (the dotted line). For each SEC run, TqLPNGh (in 50 mM HEPES (pH 7.5)/0.1 mM EDTA/0.3 M NaCl) was ten-fold diluted into the HS300e or NaCl-free He buffer (till about 4 μM protein concentration) and injected into the column equilibrated with HS300e or salt-free He buffer, respectively.

FIG. 11: The proposed mechanism of TqLPNGh fluorescence emission response towards BAI-2 binding. Dimer-to-monomer equilibrium is under control of salt and the BAI-2 concentrations. Dissociation of the dimer into monomers, induced either by BAI-2 binding or a shift to high salt concentrations, is accompanied by a large decrease in FRET (due to elimination of inter-molecular FRET). Binding of the ligand to monomeric TqLPNG (the right equilibrium) is accompanied by a small change in FRET.

FIG. 12: Accumulation of high order associates of TqLPNGh during its prolonged storage in the salt-free 50 mM HEPES (pH 7.5)/0.1 mM EDTA buffer at 4° C., examined through size-exclusion chromatography. The purified TqLPNG preparation (at ˜ 9 μM protein concentration) was stored at 4° C. in plain He buffer (containing 0.25 mM DTT and 0.25 mM TCEP) at 4° C. for about 3 weeks. 0.25 mL of the TqLPNGh solution was then mixed the equal volume of the appropriate buffer to get 0, 0.15 or 0.3 M NaCl final concentration. Following 1-2 hrs incubation (at 4° C.), the diluted protein was injected into Superdex 20010/300 column equlibrated with He, He+0.15 M NaCl or He+0.3 M NaCl buffer, respectively. The protein elution was detected through absorbance at 280 nm. The flow rate was 0.5 mL/min. The large overlapping peaks at 9-12 elution volume correspond to high order TqLPNG associates. Positions of peakes corresponding to TqLPNG dimer or monomer are marked with the arrows.

FIG. 13: Titration of TqLPNGh biosensor with autoinducer BAI-2 monitored through acceptor-to-donor emission ratio (A-to-D ratio). Measurements of TqLPNGh fluorescence emission spectra were performed in 50 mM HEPES (pH 7.5)/0.1 mM EDTA/0.4 mM boric acid//0.15 M NaCl buffer using 50 nM TqLPNGh. Emission spectra of TqLPNG were recorded by using 440 nm excitation. The values for the A-to-D ratio for the BIA-2 titration (the filled circles) were fitted to the phenomenological Hill equation to get BAI-2 concentration corresponding to the half decrease in A-to-D ratio (C_{50%}). The values for A-to-D ratio corresponding to the control titration, i.e., titration of TqLPNGh with the ligand-free plain buffer, are shown as unfilled circles.

FIG. 14: Generalized schematic of quorum sensing signaling pathways.

FIG. 15: Expression and partial purification of hTqLPNG protein fusion. (A) SDS-PAGE analysis of the hTqLPNG protein expression in E. coli BL21 (DE 3) (luxS⁻) transformed with pET21-TqLPNGh plasmid, and essential fractions in the course of hTqLPNG purification. Equal amounts of cells (as based on OD600) were loaded into the lanes 1 and 2, containing the cell culture samples prior and following IPTG induction (at 21° C. for 20 hrs), respectively. Essential fractions in the course of the hTqLPNGh purification: total suspension of the disrupted cells, 10 μL (lane 3); supernatant from suspension of the disrupted cells, 10 μL (lane 4); flow-through fraction (i.e., unbound protein species) upon loading of the soluble fraction onto the 2.5 mL Talon Metal Affinity resin column, (lane 5); the main 3 mL fraction of the 200 mM imidazole eluate, 10 μL (lane 6). 2.5 μL of each fraction, 4-fold diluted with the sample buffer, was loaded into each slot (3-6). Molecular weights of the protein ladder (the L slot) are indicated in kDa. (B) Absorption spectrum of the eluate.

FIG. 16: The map of the plasmid pET21-hTqLPNG coding for hTqLPNG.

FIG. 17: Multiple sequence alignments of LuxP protein sequences found in V. harveyi (ViHar), V. parahaemolyticus (ViPar), V. vulrnficus (ViVul), V. cholerae (Vicho), V. anguillarum (ViAng), and V. fisheri (ViFis).

FIG. 18: Structural model of monomeric TqLPNG biosensor obtained from computational protein-protein docking of both fluorescent proteins (mTurquoise2 and mNeonGreen) to LuxP. Protein backbones are shown using cartoons, and side chains residues that are part of both complex interfaces are shown in sticks. The fluorophores from both fluorescent proteins are represented as large spheres. The distance between the centers of these spheres gives the center of mass (COM) distance between the two fluorophores (black dashed line), which corresponds here to a distance of 60.6 Å. Image rendered using PyMOL.

FIG. 19: Structural model of ligand-free LuxP homodimer obtained from computational protein-protein docking. Protein backbones are shown using cartoons. Black arrows highlight the homodimerization interface that mostly occurs between the N-terminal lobes of both LuxP molecules. Image rendered using PyMOL.

FIG. 20: Structural model of dimeric TqLPNG biosensor after combining the combining the models of TqLPNG monomer and LuxP homodimer that were obtained from computational protein-protein docking. Protein backbones are shown as cartoons, and the fluorophores from all four fluorescent proteins are represented as large spheres. Both mTurquoise2 domains are found on one side of the dimer and both mNeonGreen domains are found on the other side. The almost vertical black dashed line shows an instance of intramonomer FRET (for LuxP monomer 2 in this example), while the diagonal black dashed line shows an instance of intermonomer FRET (from the donor FP of LuxP monomer 1 to the acceptor FP of LuxP monomer 2 in this example). Image rendered using PyMOL.

FIG. 21: show an exemplary diagram of the operation of TqLPNG biosensor in one preferred embodiment thereof. Here, a TqLPNG biosensor can form dimers, through the homodimerization of LuxP, at low salt concentrations and with ligand-free LuxP. High salt conditions destabilize the highly polar LuxP-LuxP dimerization interface, leading to a prevalence of TqLPNG monomers. Binding of BAI-2 to LuxP also destabilizes this interface, leading to ligand-bound TqLPNG monomers even at low salt conditions. In this preferred embodiment, the observable FRET signal from the ligand-free TqLPNG dimer comprises both intramonomer and intermonomer FRET. Binding of BAI-2 shifts towards ligand-bound TqLPNG monomer that exhibits only intramonomer FRET; the loss of intermonomer FRET would correspond to the larger decrease in the measured FRET ratio at low salt conditions.

### DETAILED DESCRIPTION OF INVENTION

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

The present invention relates to systems, methods and compositions for the detection of target molecules. In one preferred embodiment, the inventive technology may include systems, methods and compositions for the detection of QS molecules, preferably produced by bacterial pathogens or mimics thereof, and biosensors embodying such method. In general, one method of the present invention involves binding a QS compound to a fluorescent protein complex, which results in a conformational change in the complex, which causes dissociation of the biosensor dimers, and thereby yields a characteristic change in resonance energy transfer.

A biosensor protein or compound within the scope of the inventive technology may include a tripartite FRET-based fusion protein complex comprising: (1) a ligand binding domain capable of binding to a target ligand or compound and a donor-acceptor pair of chromophores moieties. In this embodiment, the ligand binding domain may be positioned between the donor and acceptor chromophore moieties, enabling fluorescent resonance energy transfer (FRET) between them as generally described herein.

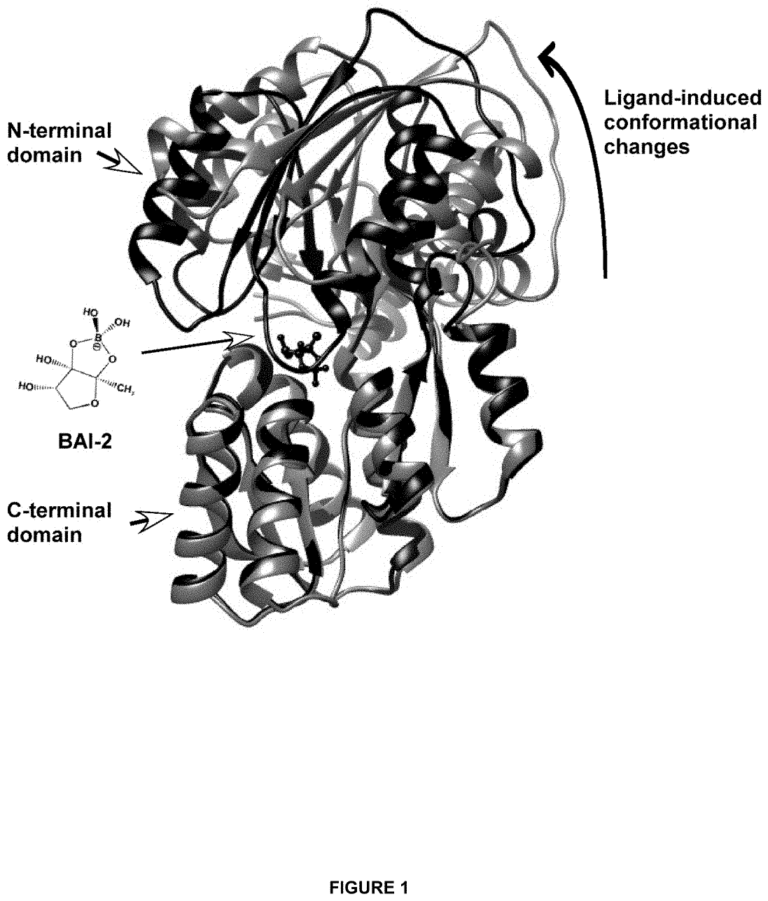

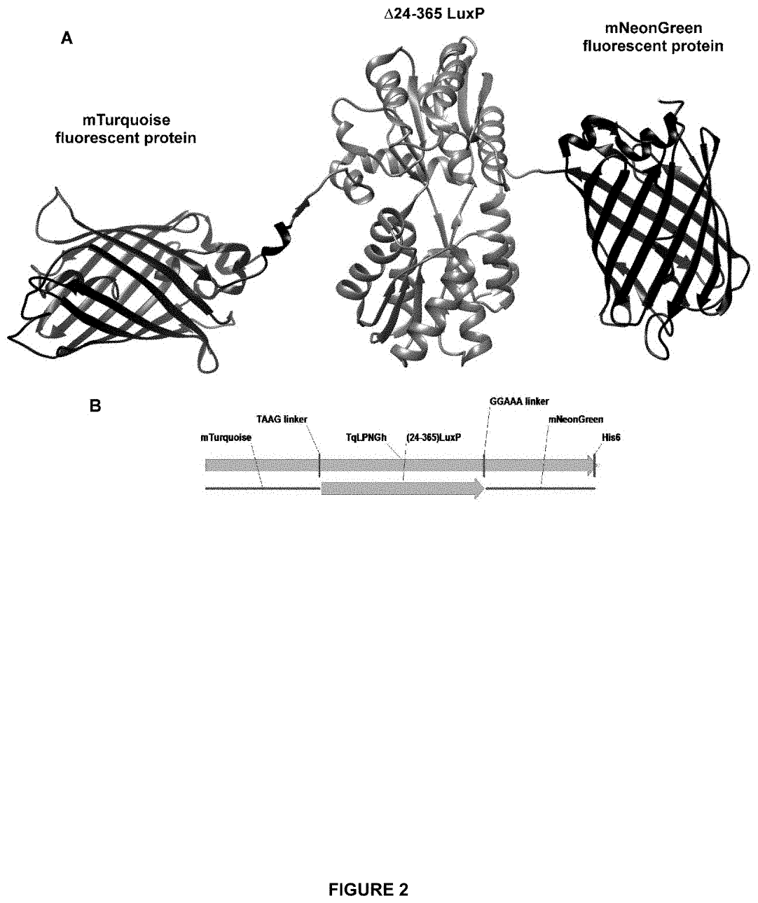

The invention may include a biosensor protein comprising: (1) a protein capable of binding to the class of QS compounds known as autoinducers, such as autoinducer 2 (AI-2) and/or derivatives thereof shown in FIG. 1; (2) a donor fluorescent chromophore; and (3) an acceptor fluorescent chromophore. Again, in this embodiment, the QS ligand binding domain or moiety is positioned between the donor and acceptor chromophore moieties, enabling fluorescent resonance energy transfer (FRET) between them as generally described herein. In one preferred embodiment shown in FIGS. 1-2, a biosensor protein may include a QS molecule ligand binding moiety, which may include a truncated (amino acids 24-365) LuxP periplasmic BAI-2 receptor from Vibrio harveyi, identified as SEQ ID No. 5. This ligand binding moiety undergoes a large structural change upon BAI-2 binding. The truncated LuxP (LP) may be fused to the mTurquoise2 (Tq, the ‘cyan emitting’ mutant variant of GFP from Aequorea victoria), identified as SEQ ID NO: 9, at its N-terminus and to the mNeonGreen (NG, the monomeric mutant variant of the green fluorescent protein derived from the Branchiostoma lanceolatum), identified as SEQ ID NO: 7 at its C-terminus. In this preferred embodiment, the above described tripartite biosensor protein may be identified as fusion protein TqLPNG or TqLPNGh, and further identified as amino acid SEQ ID NO. 2 or 3, and/or by nucleic acid SEQ ID NO. 1 generally, or any sequence having at least 70% homology with any of the above sequences.

In one embodiment, a fusion biosensor protein, such as TqLPNG, may include linker domains that tether the donor (Tq) and acceptor (NG) moieties to the QS—ligand binding moiety. In some embodiments, such linkages can be useful for positioning the Tq and NG moieties enabling FRET between their fluorophores. Typically, useful linkages can comprise relatively flexible and sterically unhindered moieties, such as glycine, alanine and polymers or copolymers thereof. Alternatively, other embodiments can include relatively inflexible linking moieties, such as amino acids having bulky side chains, e.g. phenylalanine, tyrosine, etc. Still further embodiments can comprise combinations of flexible and inflexible linking moieties, thereby achieving an intermediate degree of flexibility.

As noted above, the improved biosensor protein incorporates FRET. In general, FRET occurs when the emission spectrum of a donor moiety overlaps with the absorption spectrum of an acceptor moiety, and the donor and acceptor are close enough to electronically couple (Van der Meer et al., 1994). The spectral overlap (among the other factors like the donor radiative lifetime and the orientation factor) determines an important characteristic of the donor-acceptor FRET pair, the Forster distance (R₀), which in turn determines the dependence of the FRET efficiency (E) on the donor-acceptor distance (R):

E=R₀⁶/(R₀⁶+R⁶)

Preferably, the absorption spectra of the donor and acceptor should be well separated, so that the wavelength selected for exciting the donor (λex^{D}) minimally excites the acceptor. In other words, at λex^{D} excitation, the donor absorption should prevail over the acceptor absorption [ε_{D}(λex^{D})>ε_{A}(λex^{D})]. If the acceptor absorption at λex^{D} prevails over the donor absorption, then the directly excited acceptor emission (dirA) prevails over the acceptor emission excited through FRET mechanism (A_{FRET}), yielding a small enhancement in the acceptor emission and associated problems for the FRET evaluation.

In the case of FRET, fluorescence emission spectrum (at λex^{D} excitation) of donor-acceptor species, DA(λ) comprises the donor emission contribution, quenched (as compared with donor emission in the absence of FRET) due to the FRET reduced donor emission, and the acceptor emission contribution, enhanced (as compared with the acceptor emission directly excited at λex^{D}) due to the FRET excited acceptor:

where dirA(λ)+A_{FRET}(λ)=k[c_{DA}ε_{A}Q_{A}F_{A}(λ)+c_{DA}ε_{D}EQ_{A}F_{A}(λ)]; k is the geometric factor (which determines the fraction of the total emission collected); c_{DA}—is the molar concentration of DA-species; Q_{D}—is the donor emission quantum yield; Q_{A}—is the acceptor emission quantum yield; F_{D}(λ)—is the donor fluorescence emission spectrum; and F_{A}(λ)—is the acceptor emission spectrum.

The efficiency of FRET may be determined either from the extent of quenching of the donor emission, E=(Q_{DA}−Q_{D})/Q_{D}=1−D-DA(λ)/D-D(λ) [where D-DA(λ) is the donor emission intensity in DA-species (i.e., in the presence of acceptor); and D-D(λ)—is the donor emission in the D-species (i.e., in the absence of the acceptor)], or from the extent of enhancement in the acceptor emission, E=[A-DA(λ)/dirA(λ)−1]/[ε_{D}(λex^{D})/ε_{A}(λex^{D})] [where A-DA(λ)—is the acceptor emission component of the total emission in DA-species at λex^{D} excitation]. The latter one is preferable since the direct acceptor excitation can be determined using the same DA-species selectively excited at the acceptor excitation only, i.e., at the wavelength where the donor has no absorption (which is 505 nm in the case of TqLPNG biosensor).

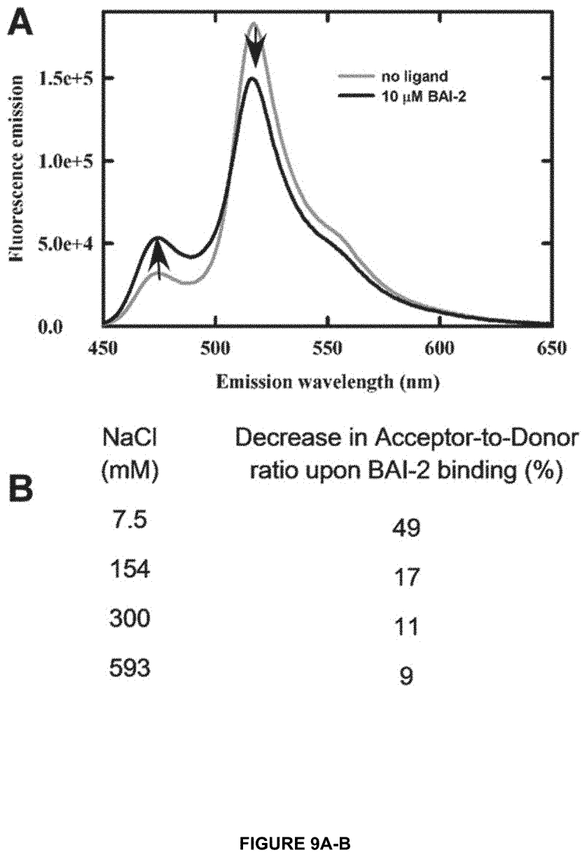

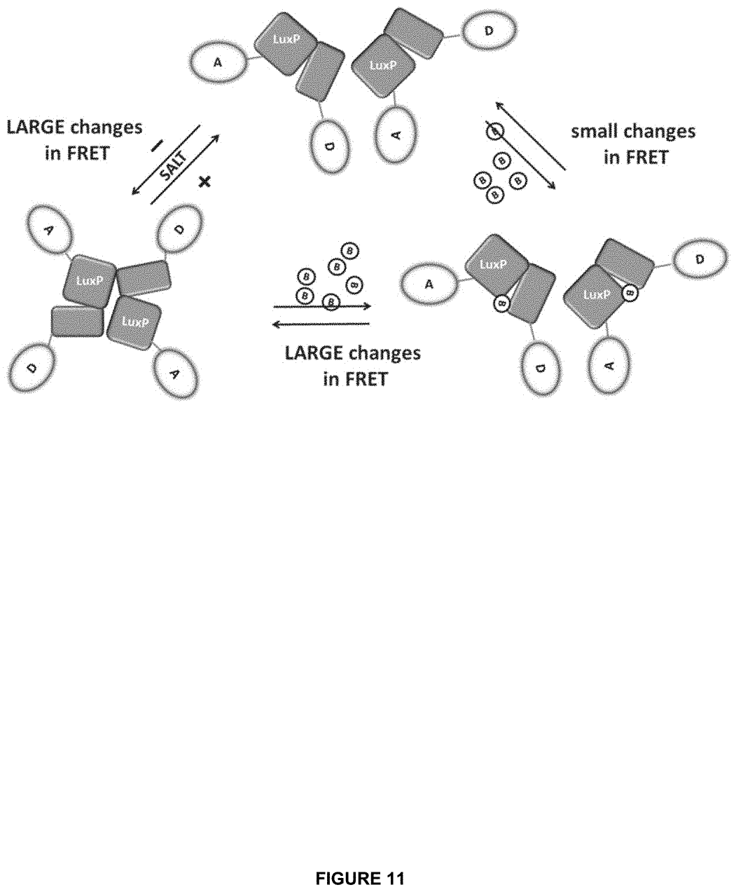

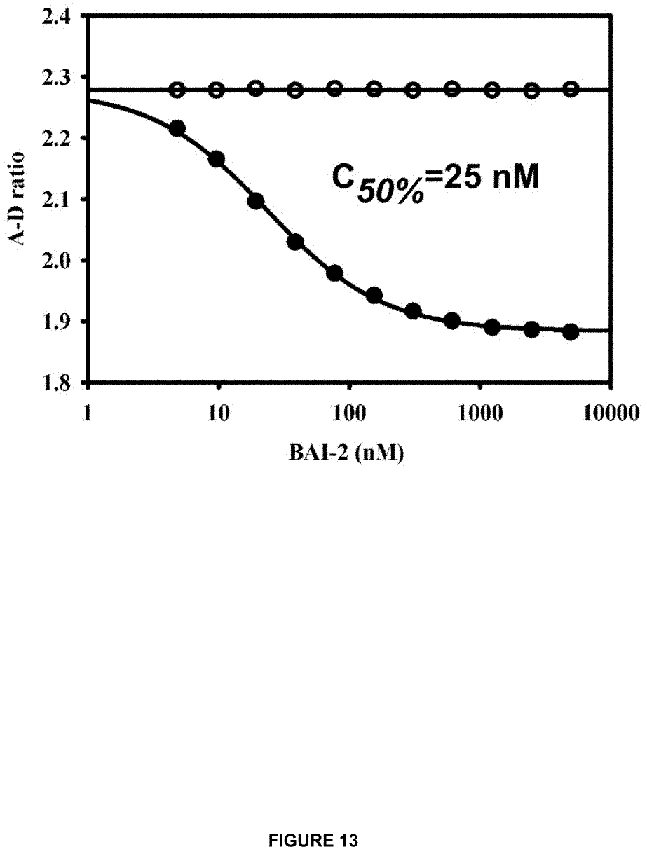

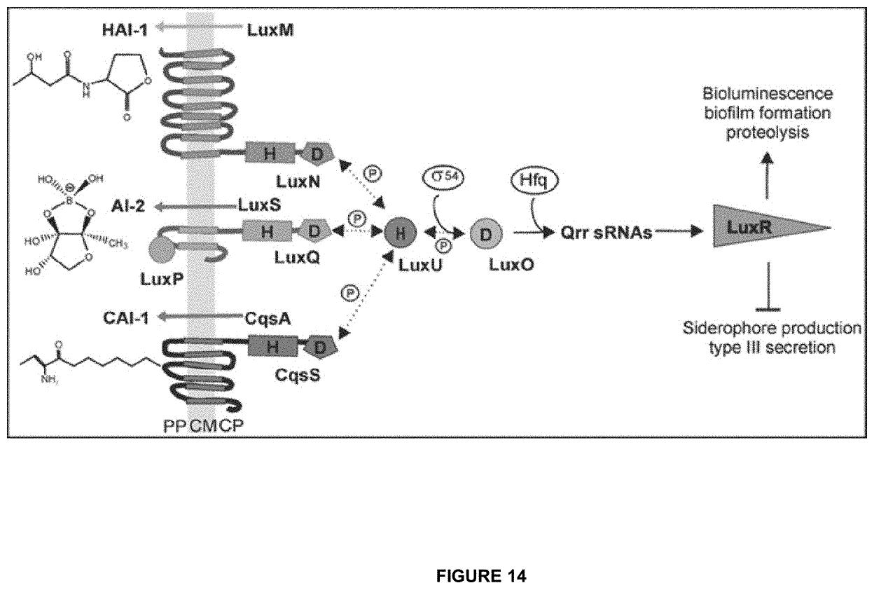

As shown in the spectra in FIG. 13, in one embodiment TqLPNG may be used as a FRET-based biosensor protein. Specifically, as shown in FIG. 11, TqLPNGh biosensor is at a salt-dependent equilibrium between monomers and dimers. The biosensor dimers feature enhanced FRET (as compared to monomeric TqLPNG) and therefore enhanced acceptor-to-donor emission ratio (again, as compared to monomeric TqLPNG). Binding of BAI-2 to TqLPNG dimers causes dissociation of TqLPNG into monomers yielding appreciable reduction in FRET (due to elimination of the intermolecular FRET component) while ligand binding to monomeric TqLPNG is accompanied by a small (if any) change in FRET within the individual biosensor. So, titration of TqLPNG solution with increasing BAI-2 concentrations is accompanied by an enhancement in donor fluorescence intensity and a corresponding decrease of the acceptor component of the TqLPNGh fluorescence emission spectrum (FIG. 9A). The above opposite changes in fluorescent intensity of D and A (which are characteristic ‘signatures’ for the decrease in FRET) result in decrease of the acceptor-to-donor emission ratio (see FIG. 13) in a concentration dependent manner, which can be used for BAI-2 quantification in biological samples.

In addition to the foregoing, FRET can be quantified by time-resolved fluorescence spectroscopy. Usually, the appropriate time-scale for such measurements falls in the nanosecond regime; however, others may fall in the pico or femtosecond parameters. In any case, an increase in FRET is indicated by a reduction in donor excited state life-time relative to an appropriate control sample. As will be apparent to one of ordinary skill in the art, there are a variety of alternative methods for quantifying FRET which may fall within the scope of the current invention.

In one embodiment, the biosensor of the present invention comprises at least the following three components: (1) a ligand binding domain protein or protein fragment, such as a full or truncated QS binding protein moiety; (2) a donor protein; and (3) a paired acceptor protein. In a preferred embodiment, a ligand binding domain protein or protein fragment ‘holds’ the donor and acceptor protein or fragments in close enough proximity for them to experience FRET. When a target ligand, such as QS molecule (or other compounds) binds the ligand binding domain, it results in a conformational change wherein the donor and acceptor move apart (due to the ligand-induced dimer dissociation) and experience less FRET. Thus, the amount of target can be quantitatively determined as a function of energy transfer associated with dimer-monomer equilibrium. Alternatively, fusion proteins of the present invention can be used to qualitatively determine the presence or absence of a target from a sample.

In one preferred embodiment, the biosensor of the present invention comprises at least the following three components: (1) a receptor protein such as a full or truncated LuxP; (2) a donor mTurquoise2 (Tq, the ‘cyan emitting’ mutant variant of GFP from Aequorea victoria); and (3) an acceptor mNeonGreen (NG, the monomeric mutant variant of the green fluorescent protein derived from the Branchiostoma lanceolatum). More particularly, the truncated LuxP binding moiety, which encompasses amino acids Δ24-365 of the LuxP protein, is bound to the Tq and NG components so that it holds Tq and NG in close proximity. This embodiment can also include one or more linkers that serve to tether Tq and NG to the truncated LuxP. Still more particularly, LuxP holds Tq and NG in close enough proximity for them to experience FRET. When the truncated LuxP protein binds to AI-2, it results in a conformational change causing dissociation of TqLPNG dimers present and yielding. Thus, the amount of AI-2 can be quantitatively determined as a function of energy transfer associated with a change in dimer-monomer equilibrium.

A fusion protein, read from the N to C terminus, may be made from Tq, truncated LuxP, and NG as shown in FIGS. 1-2, and the sequence is set forth in amino acid SEQ ID NO: 2. In this embodiment, the C-terminal tail of the biosensor fusion protein may include a His-6 tag, which may be used for purification of the fusion biosensor protein resulting from expression, for example in a genetically modified bacterium expressing a nucleic acid identified as SEQ ID NO: 1, encoding the fusion protein. In one embodiment, this expression vector may be a plasmid competent to be expressed in bacteria, such as E. coli, and may further be identified as SEQ ID NO: 10. In additional embodiments, a biosensor protein, identified as SEQ ID NO. 3, may lack a His-6 tag at its C-terminal tail.

According to well known methods, the foregoing concentration dependent FRET effect can be used to determine the amount of, in one preferred embodiment, a QS molecule such as a BAI-2 ligand, or other analyte. For instance, in one preferred embodiment, a calibration curve can be constructed by running a series of samples containing known amounts of BAI-2. Unknown concentrations can then be determined by comparison to the calibration curve.

In one embodiment the biosensor of the present invention may be used to monitor the state of an infection. In this embodiment, higher concentrations of BAI-2 generally infer the presence of larger the level of infectious pathogens. Thus, the state of infection is monitored as a function of the amount of infectious cells and/or the concentration of QS molecules in a sample, cell or a target environment. In another embodiment the present invention is used to monitor the level of QS compounds in various medical devices. According to this embodiment, higher bacterial levels result in higher QS compound levels, which can result in bacterial biofilm formation in the device and ultimately infection in the patient. Therefore, in this embodiment the present invention is used to detect the need for remedial measures, and/or check their effectiveness. In a still further embodiment, the present invention is used to identify molecular mimics of QS compounds. This embodiment can be useful in drug discovery screening protocols for drug candidates. For instance, some pharmaceutically relevant mimics of QS compounds may bind with the biosensor of the present invention.

“Fluorescent protein” refers to any protein capable of emitting light when excited with appropriate electromagnetic radiation. Fluorescent proteins include proteins having amino acid sequences that are either natural or engineered, such as the fluorescent proteins derived from Aequorea- or Branchiostoma-related fluorescent proteins.

As used herein when generally describing FRET, the “donor” or “donor moiety” or “donor protein” and the “acceptor” or “acceptor moiety” or “acceptor protein” are selected so that the donor and acceptor moieties exhibit fluorescence resonance energy transfer when the donor moiety is excited. One factor to be considered in choosing the donor/acceptor fluorescent protein moiety pair is the efficiency of FRET between the two moieties. Preferably, the efficiency of FRET between the donor and acceptor moieties is at least 10%, more preferably at least 50%, more preferably at least 80%, and more preferably at least 90% or more. The efficiency of FRET can be tested empirically using the methods described herein and known in the art, particularly, using the conditions set forth in the Examples.

“Binding protein” or “binding domain” or “binding moiety” refers to a protein or protein fragment capable of binding an analyte or ligand. Preferred binding proteins change conformation upon binding the analyte or ligand. “Analyte” or ligand refers to a molecule or ion that binds to the binding protein or domain, causing it to change conformation. Preferably, the analyte or ligand binds reversibly to the binding protein or domain.

“Moiety” refers to a radical of a molecule that is attached to another radical of the indicator. Thus, a “fluorescent protein moiety” is the radical of a fluorescent protein coupled to a binding protein moiety or a linker moiety, a “binding protein moiety” is a radical of a binding protein coupled to a fluorescent protein moiety, a “target peptide moiety” is a radical of a target peptide of the binding protein, and a “linker moiety” refers to the radical of a molecular linker that is ultimately coupled to both the donor and acceptor fluorescent protein moieties.

The term “sequence identity” or “identity,” as used herein in the context of two nucleic acid or polypeptide sequences, refers to the residues in the two sequences that are the same when aligned for maximum correspondence over a specified comparison window.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone are general examples (Schena et al. (1991) Proc. Natl. Acad. Sci. USA 88:0421).

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The term “vector” refers to some means by which DNA, RNA, a protein, or polypeptide can be introduced into a host, which may be a prokaryotic cell, such as bacteria, or a eukaryotic, such as a yeast or even animal cell. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The table below contains information about which nucleic acid codons encode which amino acids.

Amino acid Nucleic acid codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

“Peptide” refers to a polymer in which the monomers are amino acid residues which are joined together through amide bonds, alternatively referred to as a polypeptide. A “single polypeptide” is a continuous peptide that constitutes the protein. When the amino acids are alpha-amino acids, either the L-optical isomer or the D-optical isomer can be used, the L-isomers being preferred. Additionally, unnatural amino acids such as beta-alanine, phenylglycine, and homoarginine are meant to be included. Commonly encountered amino acids which are not gene-encoded can also be used in the present invention, although preferred amino acids are those that are encodable.

In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide.

As provided below, the table provides a list of exemplary conservative amino acid substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Amino Acids and Conservative Substitutes

| Amino Acid | Conservative Substitute |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Ile, Leu |
| Phe | His, Leu, Met, Trp, Tyr |
| Ser | Cys, Thr |
| Thr | Ser, Val |
| Trp | Phe, Tyr |
| Tyr | His, Phe, Trp |
| Val | Ile, Leu, Thr |

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is done using either native acrylamide gel electrophoresis or by anion-exchange HPLC as described in Pearson & Reanier, J. Chrom. 255:137-149 (1983). Additional methods are known by those of ordinary skill in the art.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

As used herein with respect to DNA, the term “coding sequence,” “structural nucleotide sequence,” or “structural nucleic acid molecule” refers to a nucleotide sequence that is ultimately translated into a polypeptide, via transcription and mRNA, when placed under the control of appropriate regulatory sequences. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences.

The term “sequence identity” or “identity,” as used herein in the context of two nucleic acid or polypeptide sequences, refers to the residues in the two sequences that are the same when aligned for maximum correspondence over a specified comparison window.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

As used herein, a compound is referred to as “isolated” when it has been separated from at least one component with which it is naturally associated. For example, a metabolite can be considered isolated if it is separated from contaminants including polypeptides, polynucleotides and other metabolites. Isolated molecules can be either prepared synthetically or purified from their natural environment. Standard quantification methodologies known in the art can be employed to obtain and isolate the molecules of the invention.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: FRET-Based TqLPNG Biosensor Protein Configuration and Signal Strategy

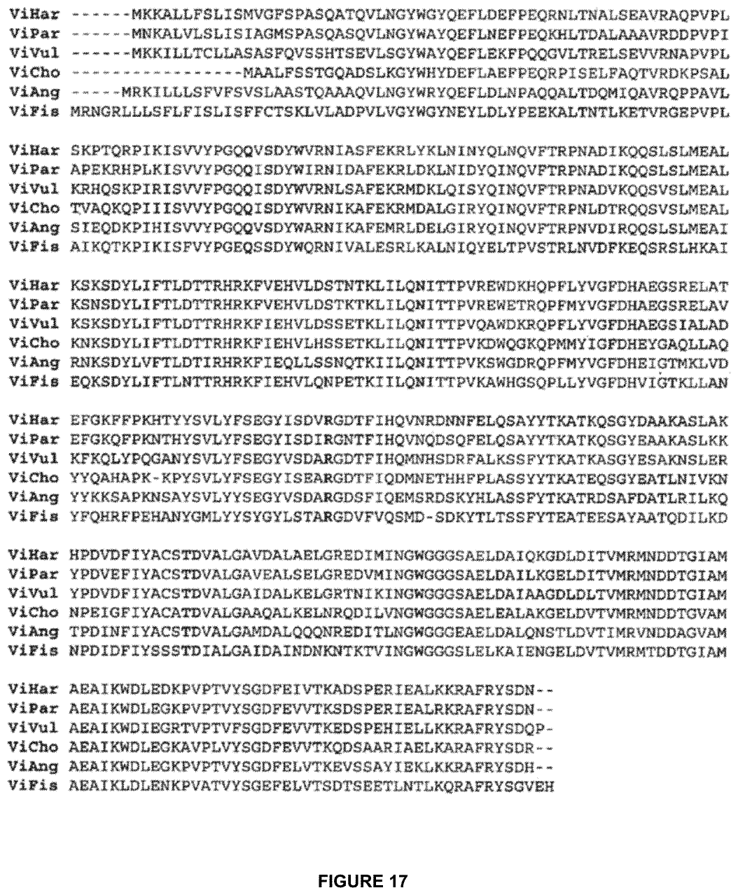

In one embodiment the biosensing fusion protein of the present invention is constructed according to the following. LuxP protein is conserved in several Vibrio species. A BLASTP search (www.ncbi.nlm.nih.gov/BLAST/) using the V harveyi LuxP protein revealed the presence of LuxP in a variety of related organisms, such as V harveyi, V. parahaemolyticus, V. vulnificus V. cholerae, and V. anguillarum. Multiple sequence alignment of LuxP sequences reveals a highly conserved amino acid sequences and BAI-2 binding residues as shown in FIG. 17. Generally, organisms with highly conserved LuxP proteins also have similarly conserved luxS genes, which are involved in production of DPD (a precursor of AI-2). Several of these organisms are known to use BAI-2 mediated QS gene regulation. In general, binding of BAI-2 with LuxP results in a structural change within the LuxP receptor and further signal transduction through a conformation selection mechanism.

In one preferred embodiment of the current inventive technology, the present inventors designed a novel TqLPNG biosensor as a tripartite fluorescent ratiometric sensor protein. The core of the TqLPNG biosensor is the N-terminus truncated (amino acids 24-365) LuxP periplasmic BAI-2 receptor from Vibrio harveyi, which undergoes large structural transition upon BAI-2 binding (See FIG. 1). As generally shown in FIG. 2A, the truncated LuxP (abbreviated further as LP) is fused to the mTurquoise2 (Tq, the ‘cyan emitting’ mutant variant of GFP from Aequorea victoria at its N-terminus and to the mNeonGreen (NG, the monomeric mutant variant of the green fluorescent protein derived from the Branchiostoma lanceolatum) at its C-terminus.

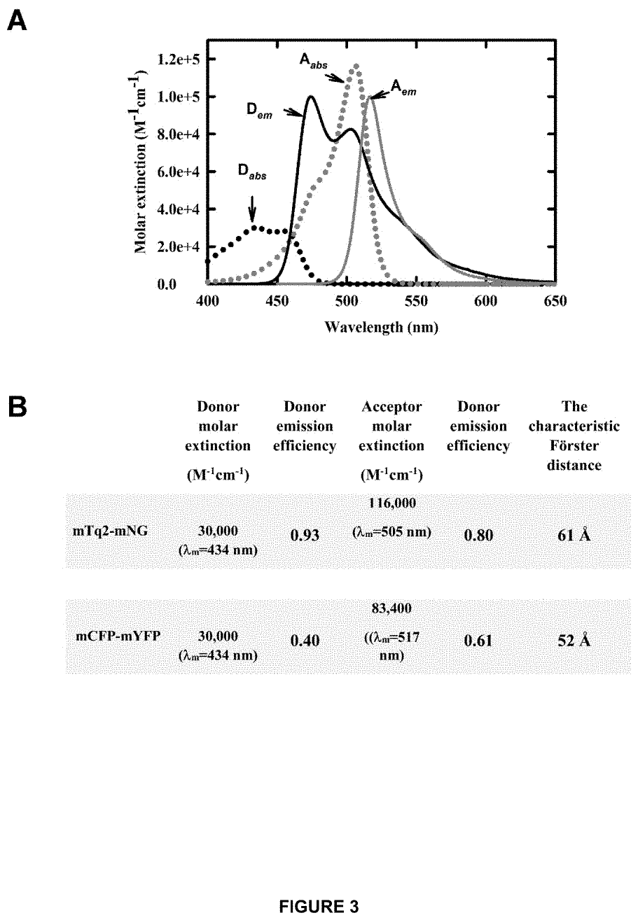

In this preferred embodiment, the present inventor's choice of Tq and NG as the donor (D) and the acceptor (A) FRET pair for the D-LP-A biosensor was based on the following enhanced properties of these fluorescent proteins (see generally FIG. 3 for the Tq and NG spectral data) including: (i) good separation of the Tq and NG emission spectra; (ii) small direct excitation of NG acceptor at the wavelength of the Tq-donor excitation (in the interval 430-440 nm), which facilitates FRET evaluation; (iii) large rotational time for both D- and A-moieties (i.e., 20-30 ns) as compared to their fluorescence lifetime (2-3 ns) increases the contribution of the D-A mutual orientation to FRET detection; (iv) as compared with the prior mCFP-mYFP donor-acceptor pair in the mCLPY biosensor described in U.S. patent application Ser. No. 11/789,479 referenced above, (see also Rajamani et al., 2007)), the Tq-NG D-A pair is much brighter that leads to significant improvements in signal clarity and detection and enables the use of the smaller biosensor concentrations; (v) Tq-NG D-A pair has a larger value for the donor-acceptor Förster distance (61 Å vs 51-52 Å for the mCFP-mYFP pair) providing higher sensitivity for the small changes in transfer efficiency at large D-to-A distances; (vi) Tq has greater photostability and lower sensitivity to variations in environment conditions (i.e., pH and salt concentration) than CFP allowing its application in a broader range of environmental samples; (vii) Tq and NG moieties have faster folding/maturation time than CFP or YFP which facilitates biosensor production.

Detection of BAI-2 by TqLPNG biosensor is based on the translation of the structural changes in LP receptor (upon BAI-2 binding) into the changes of the fluorescence spectra of its Tq and NG moieties. Specifically, under the solution conditions recommended by the inventors, a fraction of the biosensor is in the dimeric state. The changes in LP upon the BAI-2 binding causes dissociation of the TqLPNG into monomers, which is followed by the change in TqLPNG fluorescence emission spectrum. In more details, at the excited state, the fluorescent chromophore of Tq interacts with the non-excited chromophore of the NG moiety, resulting in non-radiative transfer of the excitation energy from the Tq chromophore (the donor) to the non-excited NG chromophore (the acceptor). The resulting FRET causes the distance-dependent quenching of the Tq-donor emission and concomitant enhancement of the NG-acceptor emission in the individual (monomeric) TqLPNG molecule. In the case of TqLPNG dimer, the Tq-donor features an extra quenching, and the NG-acceptor features an extra enhancement in their fluorescence emission (as compared to monomeric TqLPNG) due to the FRET from excited Tq-donor chromophore in one molecule to the non-excited NG-acceptor chromophore in the other dimer molecule. As a result, the acceptor-to-donor emission ratio of the TqLPNG dimer is markedly higher than that for TqLPNG monomer. Noteworthy, BAI-2 binding to the monomeric TqLPNG is not accompanied by significant changes in the donor-acceptor distance, thus yielding a small or no change in FRET, and an associated small change in the acceptor-to-donor emission ratio for the monomeric biosensor. Thus, the presence of a fraction of TqLPNG in the dimeric form may be especially useful for sensing of BAI-2 molecules. Furthermore, it is the fraction of dimeric TqLPNG that determines the biosensor emission response (or, in other words, amplitude) upon the BAI-2 binding (expressed as the decrease in the acceptor-to-donor emission ratio, corresponding to the fully saturated TqLPNG). In summary, the basis (modus operandi) of BAI-2 quantification is BAI-2 concentration dependent dissociation of TqLPNG dimers detected through the corresponding decrease in the biosensor acceptor-to-donor emission ratio (FIG. 11).

Accordingly, the present invention includes a biosensor fusion protein, wherein the protein is capable of producing a signal that is substantially time independent. Furthermore, time independence can be achieved in any of a wide variety of ways including, without limitation, aging, temperature treatment, sonication, absorption of electromagnetic radiation (e.g., infrared or microwave), and any combination thereof.

### Example 2: Biosensor Expression and Purification

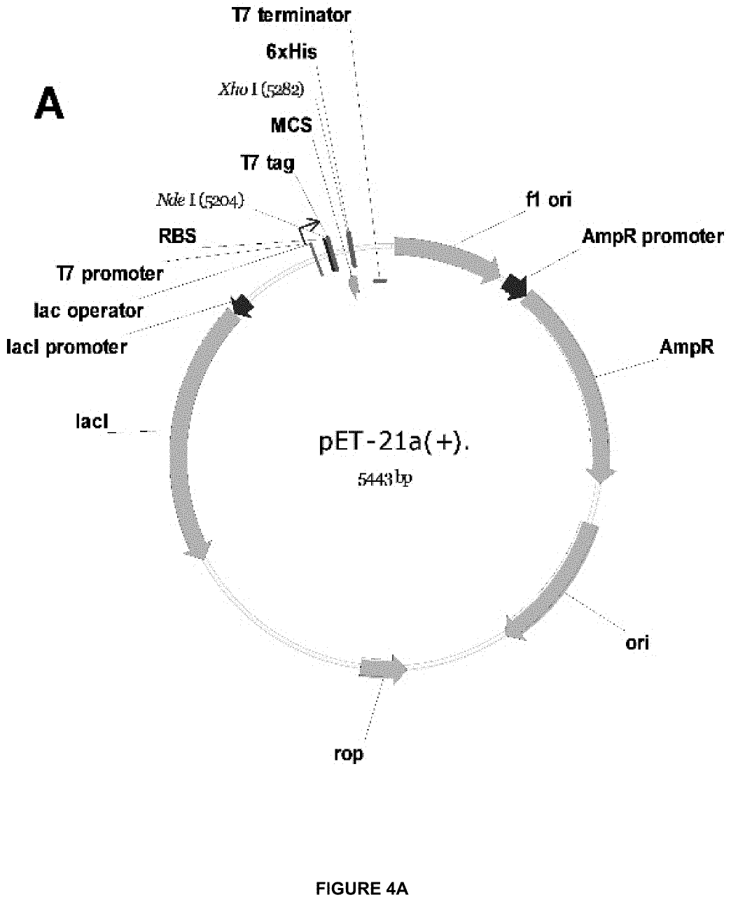

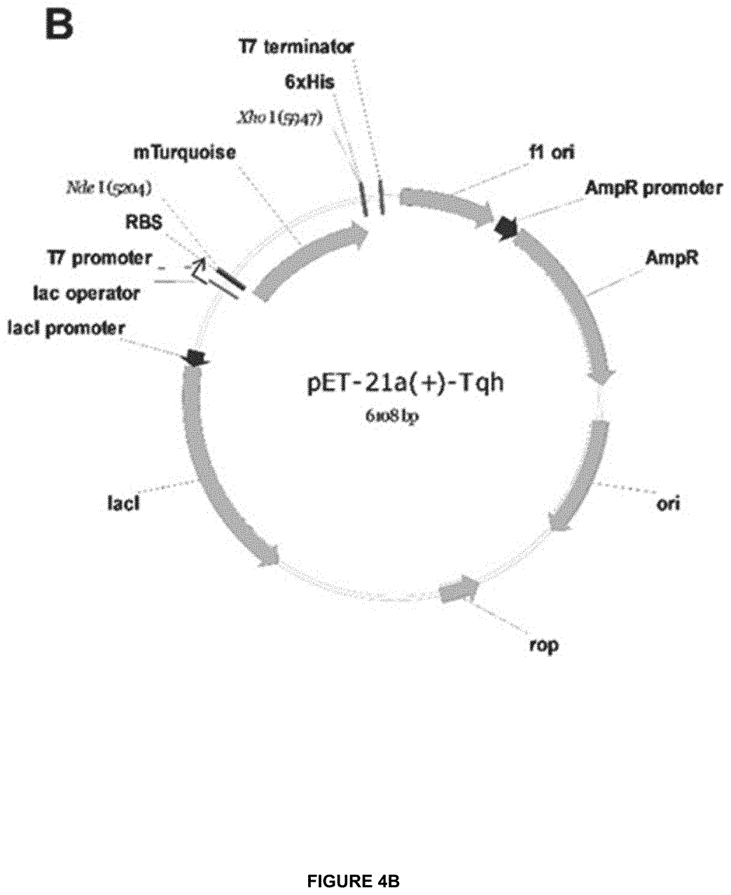

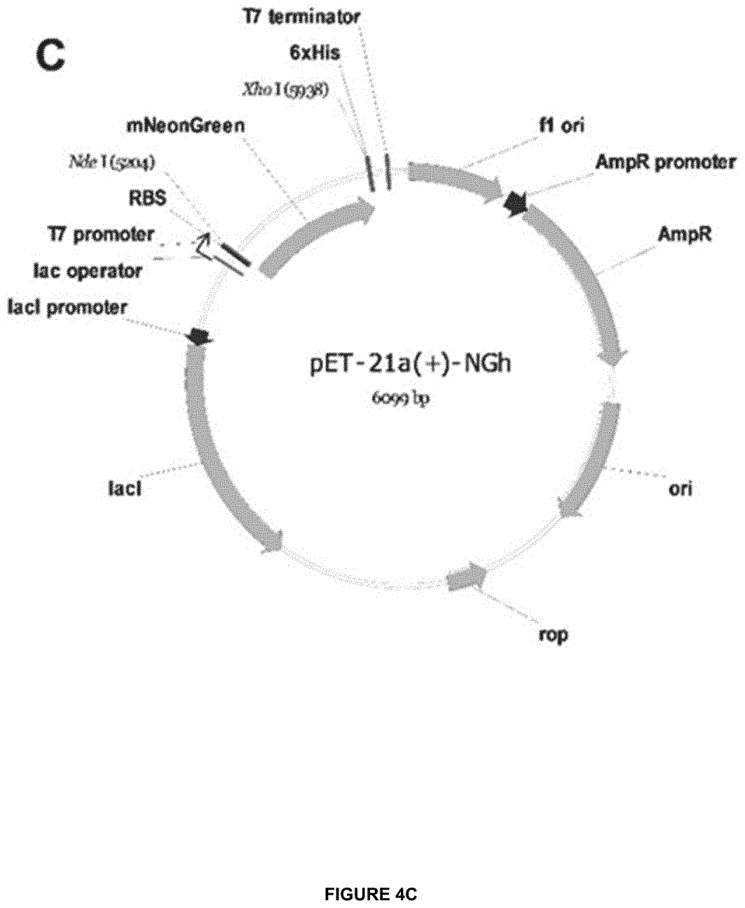

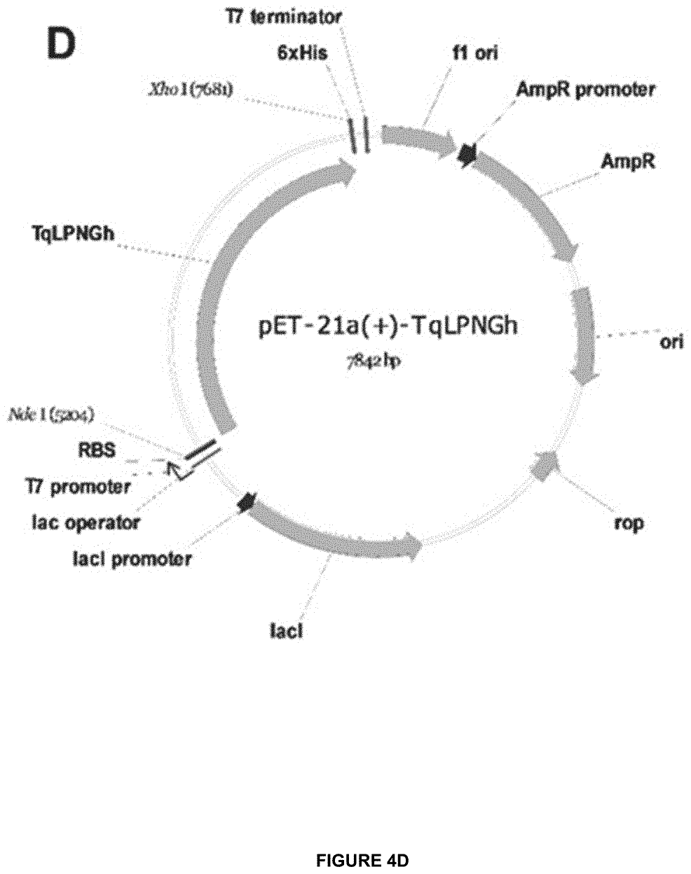

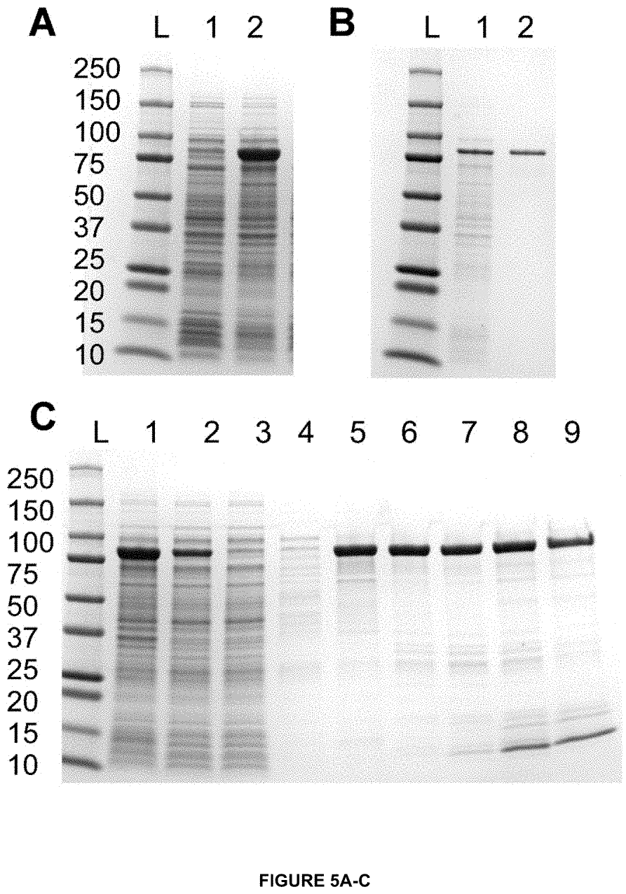

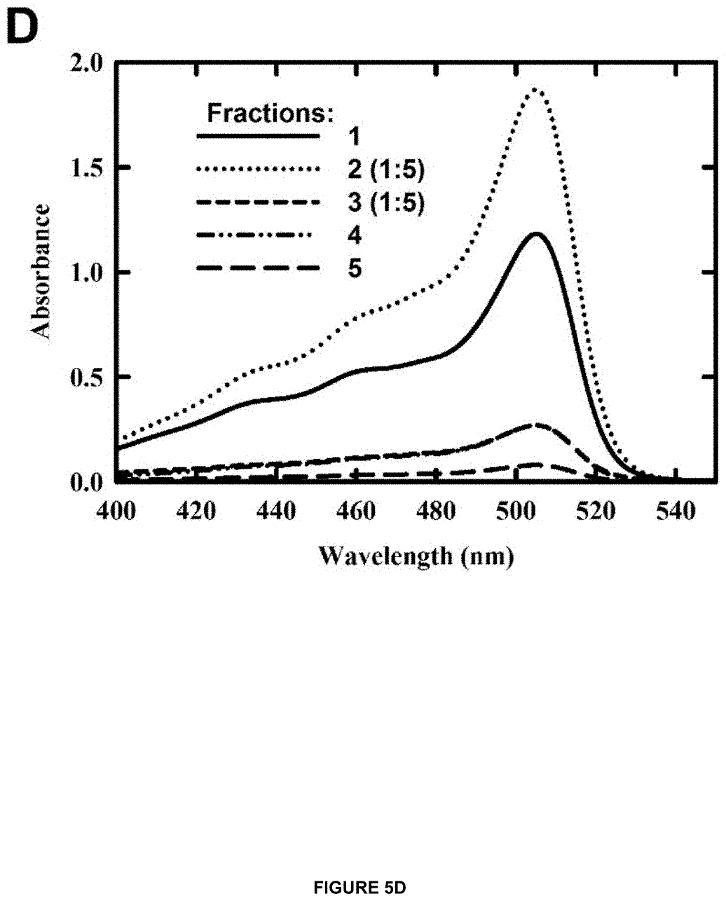

In one preferred embodiment, the TqLPNG biosensor was expressed in E. coli BL21 (DE 3) (luxS⁻) cells transformed with pET21-TqLPNGh, coding for the TqLPNG containing the His₆-affinity tag at the C-terminus of the protein fusion construct, TqLPNGh (FIG. 2A and FIG. 4D). As shown in FIGS. 5A and 5B, TqLPNGh protein expression yields approximately 200-250 mg TqLPNG from 1 L of the cell culture. As shown in FIG. 5C, although, a large fraction of the expressed biosensor was found in the insoluble fraction of the disrupted E. coli BL21 (DE 3) (luxS⁻)+pET21-TqLPNGh cells, the fraction of the biosensor in the soluble fraction was quite high (˜20% from the total TqLPNGh produced, corresponding to 50-55 mg TqLPNG from 1 L of the cell culture) and was not strongly contaminated with the cell proteins.

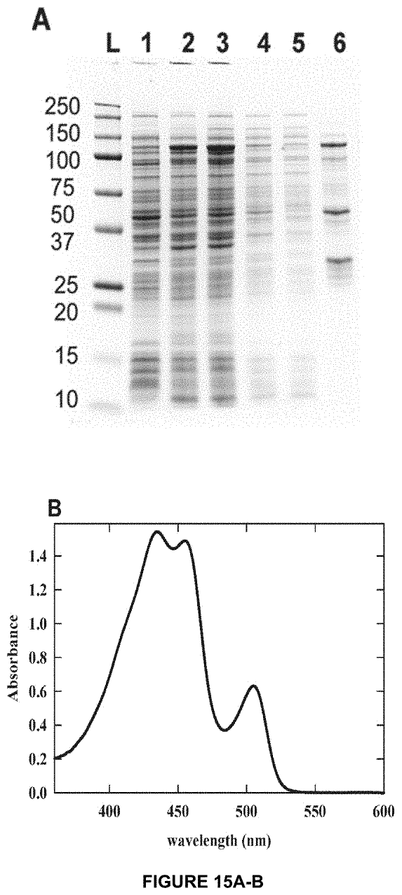

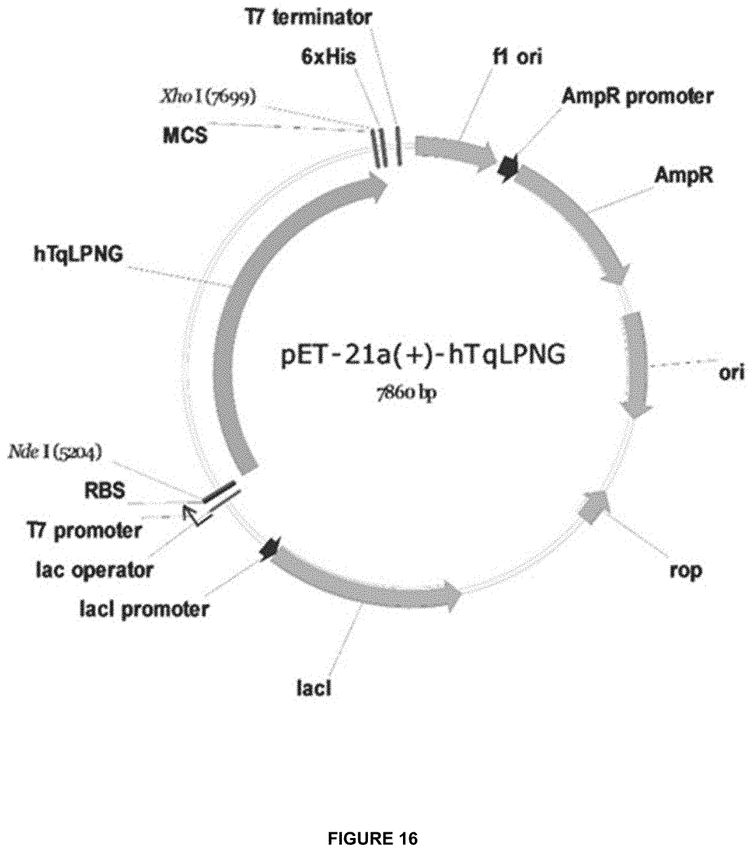

The choice of His₆-extension at the C-terminus of the TqLPNG facilitated TqLPNG purification and enabled to use specific affinity chromatography (on Talon Metal Affinity resin) which yielded efficient purification at the very first purification step (see FIGS. 5C and 5D). Of importance, the affinity purified TqLPNGh preparation was almost free from the incomplete and truncated biosensor by-products. In contrast, as demonstrated in FIG. 15, after similar purification, N-His tagged variant of hTqLPNG protein was heavily contaminated with truncated hTq and hTqLPNG fragments.

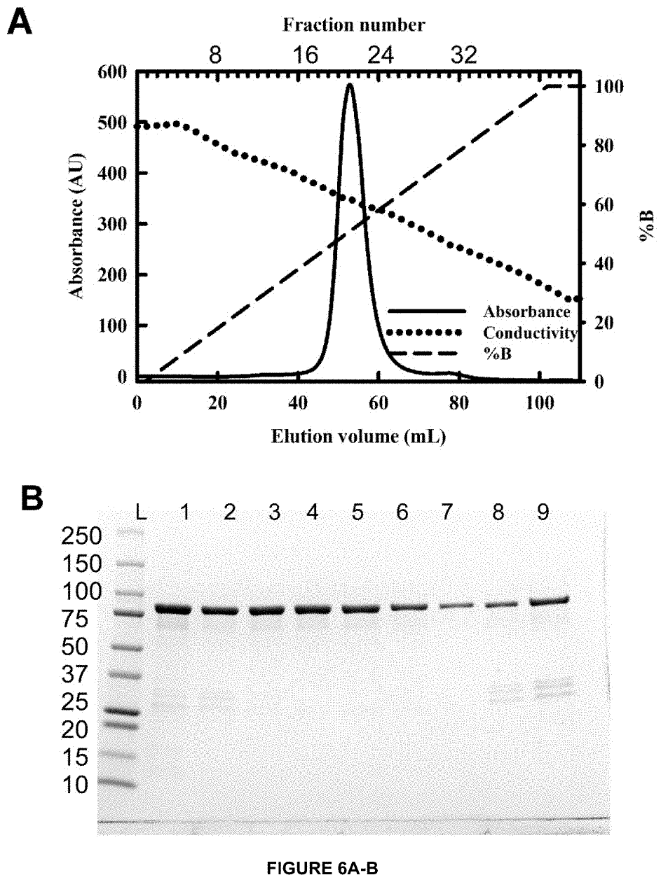

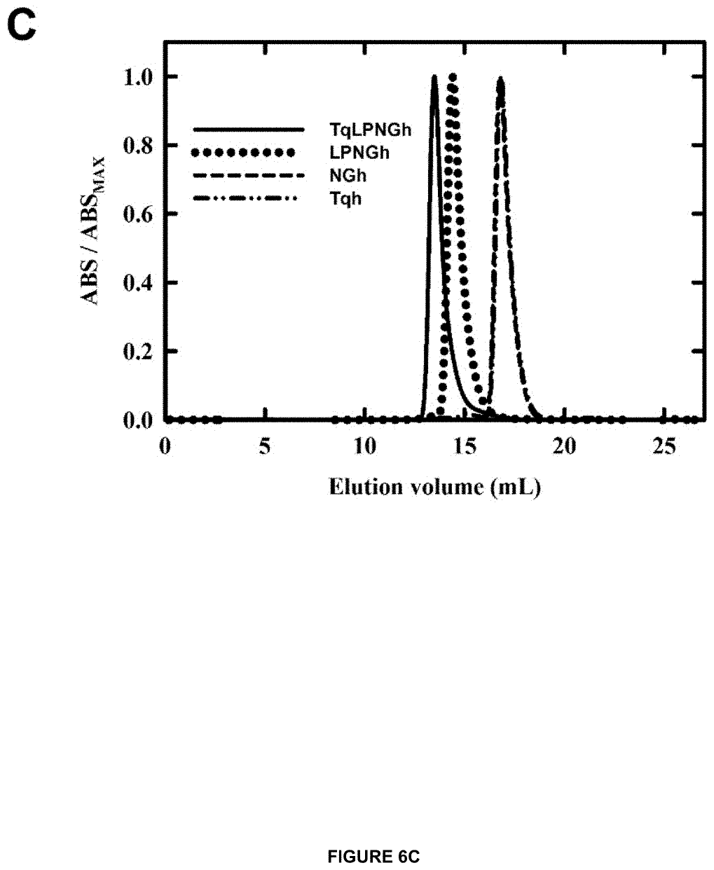

Further purification of TqLPNGh through hydrophobic interaction chromatography (HIC) on HiTrap HP Butyl column yielded nearly homogeneous biosensor preparation (FIGS. 6A and 6B). The HIC purified TqLPNGh had a single peak elution profile in size-exclusion chromatography [performed in 50 mM HEPES (pH 7.5)/0.3 M NaCl/0.1 mM EDTA] corresponding to a 90-100 kDa protein (as expected from TqLPNGh molecular weight, 93.0 kDa). Thus, no further purification was required. Following HIC, TqLPNGh was concentrated and converted into the 50 mM HEPES (pH 7.3)/0.3 M NaCl/50% (v/v) glycerol/˜0.6 mM EDTA buffer for storage at −20° C. The yield of the apparently homogeneous TqLPNGh was about 35 mG from 1 L of the cell culture.

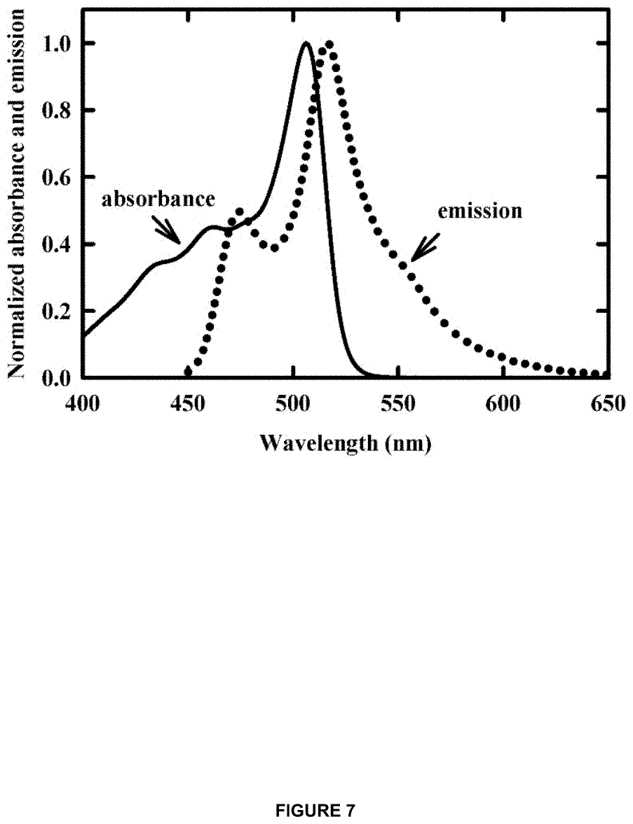

Absorbance and fluorescent emission spectra of the purified TqLPNGh biosensor are presented in FIG. 7. Both spectra exhibit characteristic peaks specific for individual Tq and NG fluorescent proteins. The fluorescence emission of the TqLPNGh has distinctive Tq-donor and NG-acceptor components well separated for further FRET-based application in BAI-2 binding assay as described generally herein.

Notably, the presence of sufficiently high concentration of NaCl (i.e., 0.3 M) in all buffers used for TqLPNGh purification (and storage, especially) is very important, since it prevents accumulation of high order TqLPNG associates.

### Example 3: Mechanism of TqLPNG Action as a Biosensor for BAI-2

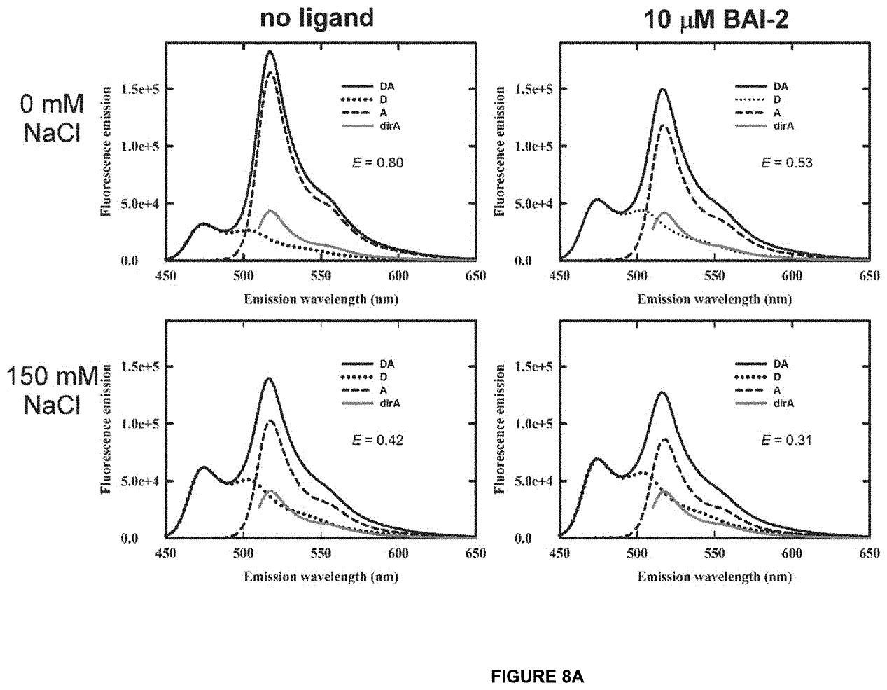

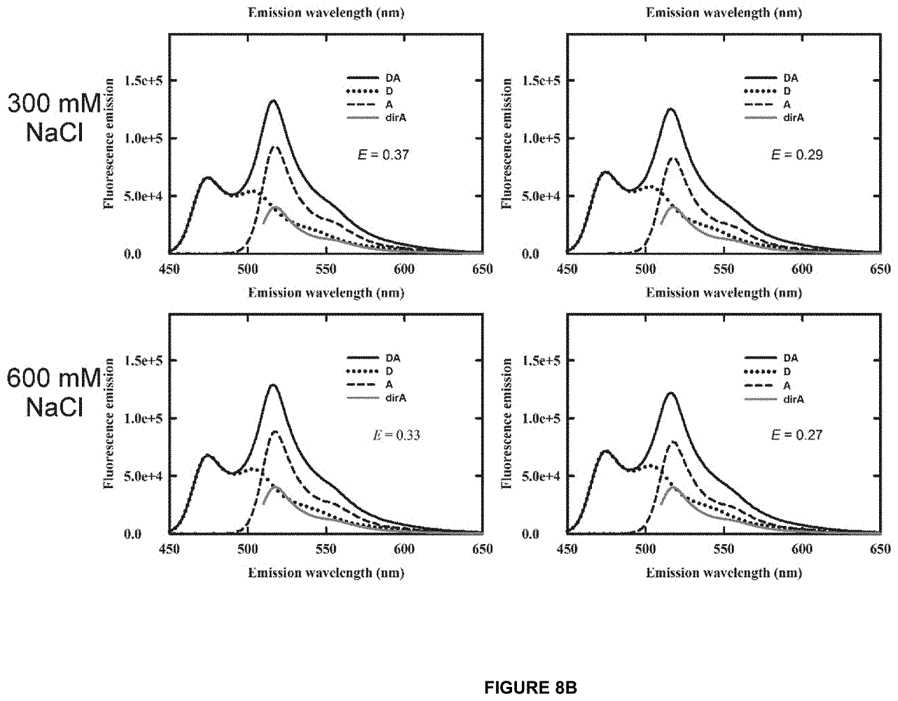

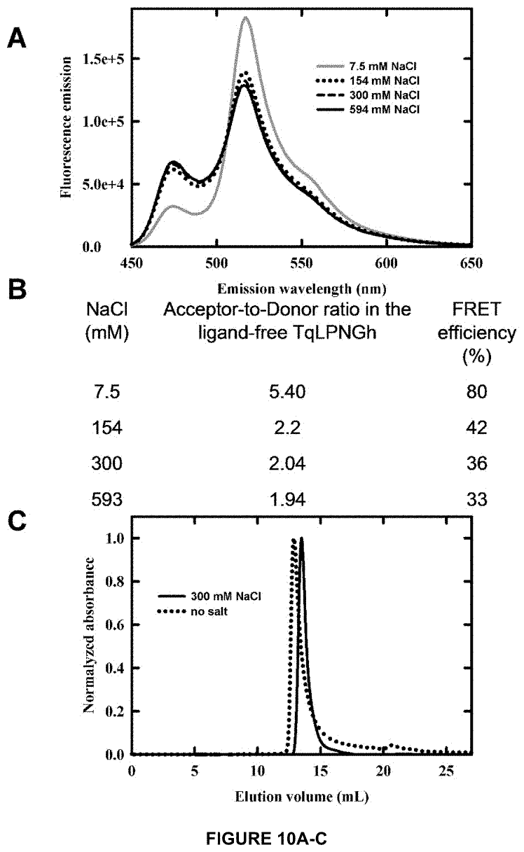

TqLPNG fluorescence response towards BAI-2 binding was examined at different concentrations of NaCl (see FIG. 8). At any salt concentration, BAI-2 binding yielded an increase in the biosensor donor emission and the corresponding decrease in the acceptor emission, originated from the ligand-induced decrease in FRET between the donor and the acceptor chromophores. Indeed, for any salt concentration BAI-2 binding caused a decrease in FRET efficiency (follow the E-values in FIG. 8) and associated decrease in the acceptor-to-donor emission ratio. The decrease in the acceptor-to-donor emission ratio corresponding to the fully saturated TLPNG, i.e., the observable amplitude, was strongly dependent on the salt concentration (see FIG. 9). The observable BAI-2 binding amplitude was maximal at low salt buffer (−49%), strongly dropped at 0.15 M and became too small (for BAI-2 quantification applications) at 0.3 M and higher NaCl concentrations. Taking into account that at 0.3 M NaCl TqLPNG is apparently monomeric (see FIG. 10C), one may conclude that BAI-2 binding to monomeric TqLPNG is accompanied by quite subtle (if any) decrease in FRET (and associated small decrease in the acceptor-to-donor emission ratio).

The nature of the above noted strong salt dependence in the observable binding amplitude was clarified by simultaneous measurements of ligand-free TqLPNG fluorescence emission spectra. The experimental data on ligand-free TqLPNG are summarized in the FIG. 10. Response of TqLPNG emission spectrum towards decreasing salt concentrations (from 0.3-0.6 M NaCl concentrations, corresponding to essentially monomeric TqLPNG, as follows from its SEC elution profile at 0.3 M NaCl, shown in FIG. 10C) corresponds to the FRET increase (and associated increase in the acceptor-to-donor emission ratio), which is markedly sharp when switching from 0.15 M NaCl to low salt buffer. The most reasonable interpretation of the sharp increase in FRET for the ligand-free TqLPNG is association of the monomeric TqLPNG yielding enhanced FRET due to extra inter-molecular FRET (on top of the FRET within the monomeric biosensor). This interpretation was further supported by SEC for the ligand-free TqLPNG performed at low salt buffer (see the dotted line on FIG. 10C). The low-salt elution profile of TqLPNG was shifted to the smaller elution volume (as compared to that for the monomeric TqLPNG at 0.3 M NaCl) and approximately corresponded to TqLPNG dimers.

Based on the above experiments, the inventors propose the following simplified model for the TqLPNG emission response towards BAI-2 binding schematically shown in FIG. 11. TqLPNG has a monomer-to-dimer equilibrium, which is strongly dependent on environment salt concentration. Specifically, high salt shifts the equilibrium towards the monomeric state, while low salt favors the dimeric state. The dimeric state features enhanced FRET (due to extra inter-molecular FRET) as compared to the monomeric state. So, transferring of TqLPNG to low (or lower) salt conditions leads to increased FRET. Binding of the BAI-2 to monomeric TqLPNG does not significantly affect donor-to-acceptor distance and/or donor-acceptor mutual orientation yielding small (if any) change in FRET for the monomeric biosensor. In contrast, BAI-2 induced structural changes cause perturbations within the dimer interface yielding dissociation of a dimer into monomer, which is accompanied by a large change in FRET (and associated decrease in the acceptor-to-donor emission ratio). Thus, the presence of a fraction of TqLPNG in the dimeric form may be especially important for sensing of BAI-2 molecules. Furthermore, it is the fraction of dimeric TqLPNG that determines the biosensor emission response (or, in other words, amplitude) upon BAI-2 binding (expressed as the decrease in the acceptor-to-donor emission ratio, corresponding to the fully saturated TqLPNG). In summary, the basis (modus operandi) of BAI-2 quantification is BAI-2 concentration dependent dissociation of TqLPNG dimers detected through the corresponding decrease in the biosensor acceptor-to-donor emission ratio.

### Example 4: FRET Based BAI-2 Bioassay

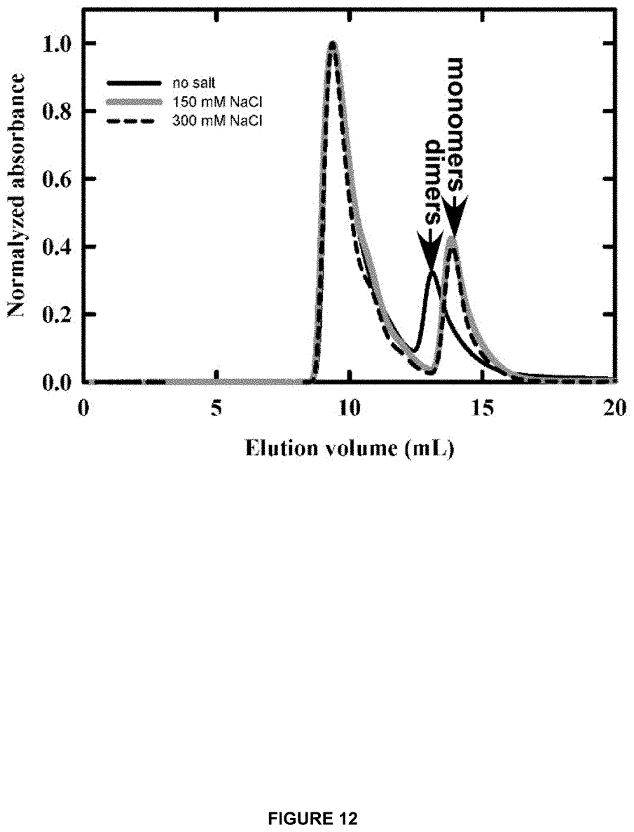

The reasonable question that follows deciphering the origin of the TqLPNG fluorescence emission response towards BAI-2 binding is to understand the optimal conditions for the biosensor-based determination of BAI-2 concentrations. Low NaCl conditions provide an increased change in acceptor-to-donor emission ratio for the BAI-2 binding favorable for accurate BAI-2 quantification. However, prolonged storage of TqLPNG in low salt buffer (even at low temperature, i.e., 4° C.) yields accumulation of high-order TqLPNG associates which do not respond to BAI-2 binding (most likely due to enhanced stability of high order associates towards dissociation) (see FIG. 12). Thus, there should by a compromise between the binding amplitude of the observable signal (i.e., the decrease in the acceptor-to-donor emission ratio corresponding to fully saturated biosensor) and stability of the TqLPNG preparation (against the formation of high order associates). So, at 0.3 M NaCl there is no formation of ‘inactive’ high-order associates for prolonged storage at 4° C., yet the fraction of dimer species and associated ligand binding amplitude are too small to get accurate BAI-2 quantification. A good compromise is the use of HEPES (pH 7.5) buffer containing 0.15 M NaCl, which provides both a good stability and acceptable binding amplitude.

To verify the suitability of purified TqLPNGh as biosensor and the suggested assay conditions (i.e., the optimal NaCl concentration), the present inventors evaluated the effect of BAI-2 on the biosensor emission spectrum in 50 mM HEPES (pH 7.5)/0.4 mM boric acid/0.1 mM EDTA buffer containing 0.15 M NaCl. They found that titration of TqLPNGh solution with increasing BAI-2 concentrations was accompanied by an enhancement in donor fluorescence intensity and corresponding quenching of the acceptor component of the TqLPNGh fluorescence emission spectrum. Opposite changes in fluorescent intensity of D and A result in decrease of the acceptor-to-donor emission ratio (see FIGS. 9A and 12). The pattern of the changes was consistent with the expected decrease in the D-to-A FRET efficiency associated with dissociation of TqLPNG dimers present induced by the ligand binding. The magnitude of the observed changes (˜17%) is sufficient for the accurate determination of BAI-2 concentration in environment samples.

To determine the sensitivity of the suggested TqLPNG-based BAI-2 assay, the present inventors determined both BAI-2 concentration corresponding to the half-transition (C_{50%}) in the TqLPNG emission response under the suggested assay conditions (50 nM TqLPNG, 50 mM HEPES (pH 7.5)/0.1 mM EDTA/0.15 M NaCl buffer containing 0.4 mM boric acid) and the BAI-2•TqLPNG dissociation constant under the suggested assay conditions (i.e., 50 nM TqLPNG, 50 mM HEPES (pH 7.5)/0.1 mM EDTA/0.15 M NaCl buffer containing 0.4 mM boric acid) (see FIG. 13). The values for D-to-A fluorescence ratio values BAI-2 concentration were plotted and then fitted with the phenomenological Hill equation or with the simple single site binding model. The fit values were C_{50%}=24±1 nM and K_{d}=9±4 nM. The presence of boric acid in the assay buffer (i.e., 50 mM HEPES (pH 7.5)/0.1 mM EDTA/0.15 M NaCl/0.4 mM boric acid) may be beneficial. Deficiency in boric acid may cause strong reduction in the apparent ligand affinity, yielding enhanced K_{d}-value and, therefore, a strong decrease in biosensor sensitivity.] Thus, the suggested assay revealed a great sensitivity for BAI-2. The suggested BAI-2 assay demonstrates the improved utility of the TqLPNGh protein construct as a biosensor for determination of BAI-2 concentration. Indeed, the TqLPNG biosensor revealed more than 10 fold higher sensitivity towards BAI-2 concentration as compared to the mCLPY biosensor reported by Rajamani (10 nM for the BAI-2•TqLPNG dissociation constant versus 270 nM for the mCPLY).

### Example 5: Molecular Modeling of the TqLPNG Biosensor

To investigate the molecular mechanisms of function for the TqLPNG biosensor, a computational technique called protein-protein docking was used to build a molecular model of this protein construct. The main questions addressed using this approach are whether either or both fluorescent proteins (FPs) form a complex with LuxP in this biosensor, and whether these complex/es are driven mainly by salt-bridge and other polar contacts. Docking of the two FPs to LuxP suggested that both make a tight complex with separate lobes of LuxP (FIG. 17). In this molecular model, mTurquoise2 interacts mainly with the C-terminal lobe of LuxP with a predicted binding affinity of around 1.1 nM. The other FP, mNeonGreen, interacts mainly with the N-terminal lobe of LuxP with a predicted binding affinity of around 5.9 nM. Both LuxP-FP complexes have an extensive interaction interface that comprise almost an almost equal mix of polar (i.e. hydrophilic) and nonpolar (i.e., hydrophobic) contacts. Note that the C-terminus of mTurquoise2 and the N-terminus of mNeonGreen are connected in this model to the N- and C-termini, respectively, of LuxP (via the corresponding linkers) such that the correct domain order of the biosensor construct is maintained (mTurquoise2-LuxP-mNeonGreen).

The predicted distance between the center of masses (COMs) of the fluorophores from both FPs is around 60.6 Ain the molecular model containing ligand-free LuxP (black dashed line in FIG. 17). Upon binding of the BAI-2, the ligand-induced conformational transition in LuxP does not lead to any steric clashes with either FP and thus both FPs remain bound to their respective lobes from LuxP. The conformational change in LuxP upon BAI-2 binding alters the inter-COM distance between both fluorophores by around 2 Å, which corresponds to a relatively small fractional change in FRET of around 10% for this FP pair. This would correspond to a relatively small change in the observable output amplitude if this FRET change is used as a measurable signal.

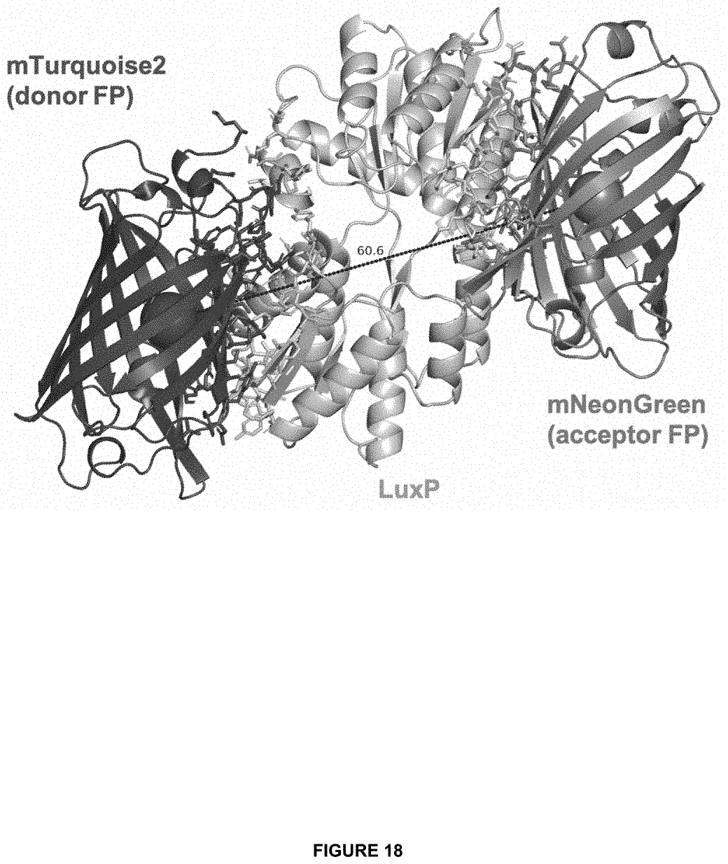

Because of the observed salt-dependent formation of dimers in experiments, protein-protein docking was used to investigate the molecular mechanism leading to this dimer formation. Since monomer-inducing mutations are present in both FPs, the focus here was on the possible homodimerization of LuxP. Docking of two LuxP molecules suggested that a tight symmetric homodimer interface is formed between the N-terminal lobes (FIG. 18). The predicted binding affinity for this LuxP-LuxP interface was around 6.7 nM. Due to the ligand-induced conformational changes in LuxP upon binding of BAI-2, the predicted binding affinity of the LuxP homodimer decreases by around four orders of magnitude to around 64 μM. Unlike the binding interfaces between LuxP and either FP, the LuxP homodimer interface comprises mostly salt bridges and other polar contacts (˜87% polar vs. ˜13% nonpolar). The highly hydrophilic interface of the LuxP homodimer can account for the observed prevalence of monomers at high salt concentrations (due to increased ionic screening of polar residues leading to reduced formation of contacts) and the presence of dimers at low salt concentrations.

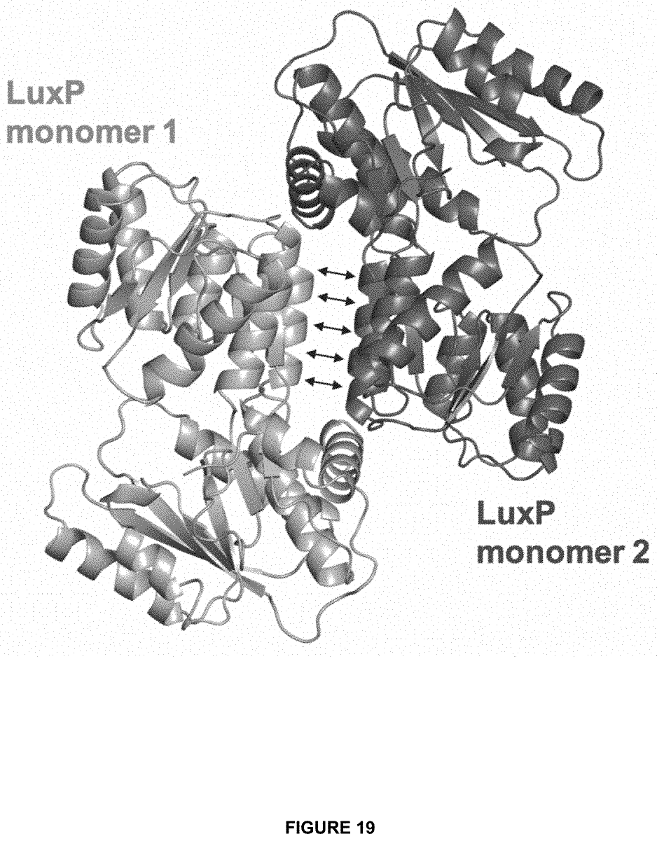

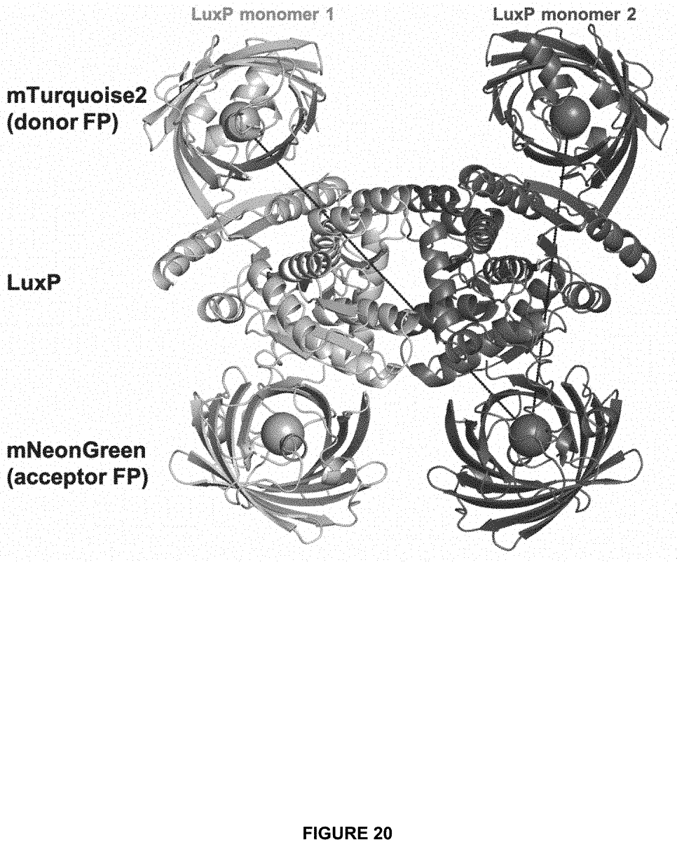

The structural model for the TqLPNG biosensor dimer is given in FIGS. 19-20. The FPs from one monomer does not exhibit steric clashes with FPs from the other monomer. This holds for both the ligand-free and ligand-bound states of LuxP in the biosensor dimer. Note that both mTurquoise2 domains are located on one side of the biosensor dimer, while both mNeonGreen domains are found on the other side. The predicted inter-COM distance between the donor fluorophore on one monomer and the acceptor fluorophore from the other monomer is around 77.6 Å. The biosensor dimer will thus exhibit intermonomer FRET in addition to intramonomer FRET for an overall larger observable and measurable signal. As noted above, either binding of BAI-2 ligand to LuxP or high salt conditions will induce the formation of monomeric biosensor (due to destabilization of the LuxP homodimer interface) that exhibits only intramonomer FRET.

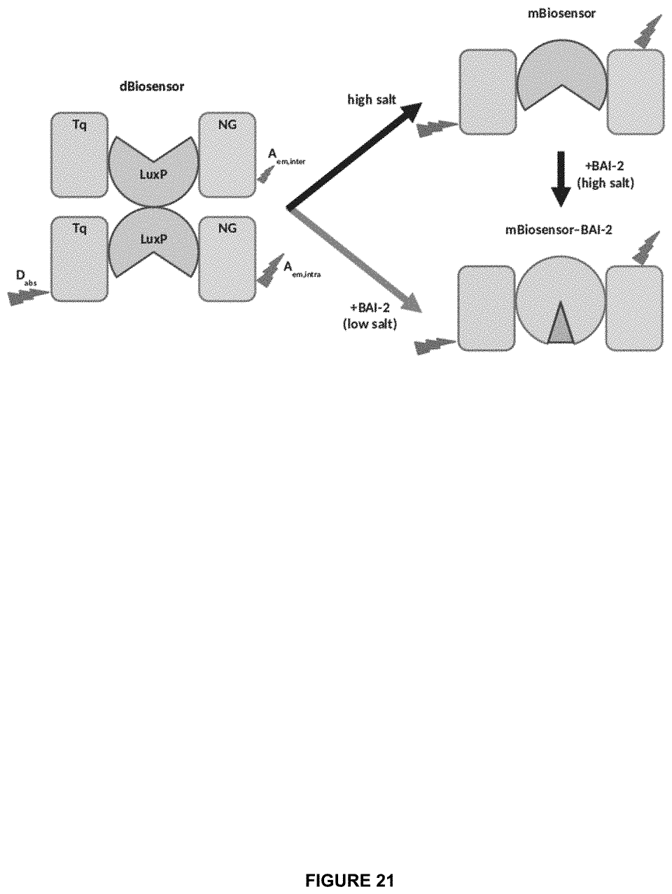

As highlighted in FIG. 21, in a preferred embodiment, the TqLPNG biosensor can form dimers, through the homodimerization of LuxP, at low salt concentrations and with ligand-free LuxP. High salt conditions destabilize the highly polar LuxP-LuxP dimerization interface, leading to a prevalence of TqLPNG monomers. Binding of BAI-2 to LuxP also destabilizes this interface, leading to ligand-bound TqLPNG monomers even at low salt conditions. In this preferred embodiment, the observable FRET signal from the ligand-free TqLPNG dimer comprises both intramonomer and intermonomer FRET. Binding of BAI-2 shifts towards ligand-bound TqLPNG monomer that exhibits only intramonomer FRET; the loss of intermonomer FRET would correspond to the larger decrease in the measured FRET ratio at low salt conditions. Note that at high salt conditions, the biosensor exists predominantly as a monomer in either the presence or absence of bound ligand. Due to the small shift in the distances between fluorophores upon binding of ligand in the biosensor monomer, a smaller decrease in the measured FRET ratio would be observed at high salt conditions.

### Example 6: Experimental Material and Methods

Materials

(4S)-4,5-Dihydroxy-2,3-pentadion (DPD) known as autoinduer-2 (AI-2) was purchased from Omm Scientific (Texas) as 4.47 mM solution in 0.5 mM NaHSO₄ (pH 1.56) and stored frozen at −80° C. unless required.

Strains and Plasmids Design and Construction

All the strains and plasmids used in this invention are listed below in Table 1. All plasmids were made by GENESCRIPT (NJ, USA) although any generic bacterial plasmid may be substituted for the expression of the biosensor protein. As generally shown in FIG. 4, plasmid descriptions for certain preferred embodiments are as follows:

As generally shown in FIG. 4A, the exemplary expression vector pET21-TqLPNGh encodes full length biosensor insert that consists of DNA sequences encoding the monomeric Turquoise fluorescent protein (mTurquoise2) linked to the LuxP receptor and the monomeric Neon-Green fluorescent protein. Sequences of fluorescent proteins were codon-optimized for E. coli expression using the SnapGene program suit. Resulting sequence was ordered to be inserted into pET21a plasmid between NdeI and XhoI restriction sites. As result, in this embodiment the biosensor construct is expressed under the control of the T7 promoter as six-histidine-tagged fusion protein.

As generally shown in FIG. 4B, the exemplary expression vector pET21-Tqh—was used for expression of control donor fluorescent protein (donor only). It was constructed as generally described above, but in this embodiment a monomeric Turquoise fluorescent protein gene was inserted into a pET21a expression vector. Thus, in this embodiment the Turquoise gene is under control of T7 promoter and could be expressed as a His6 fusion protein.

As generally shown in FIG. 4C, the exemplary expression vector pET21-NGh—was used for expression of the control acceptor fluorescent protein (acceptor only). It was constructed as generally described above, but in this instance a monomeric NeonGreen fluorescent protein gene was inserted into a pET21a expression vector. Thus, in this embodiment the protein gene is under control of T7 promoter and is expressed as His-tagged fusion protein.

Protein Expression

In one preferred embodiment, E. coli BL21 (DE 3) (luxS⁻) were transformed with the pET21-TqLPNGh plasmid by using home-made electrocompetent cells and a standard protocol (Maniatis et al., 1982). 1 μL of 10 ng/μl plasmid was added to 50 μL suspension of competent cells. Following the transformation and post-transformation growing of the cells in 1 mL SOC medium, the cells were plated onto LB-agar plate (containing carbenicillin 50 mg/L) and incubated at 37° C. for 16 h. Next day, bacterial colonies from the LB-agar plate were washed out from the plates using 10 mL LB, and the wash was used for inoculation of 0.5 L LB-carbenicillin medium (in 2 L conical flask). The culture was grown at 28° C. with aeration (200 rpm) till OD600˜ 0.6, and then transferred into a refrigerated shaker at 21° C. (200 rpm) for about 40 min to cool it down prior to induction with 1 mM IPTG. Following IPTG addition, the culture was further grown at 21° C. for 20 h, and then transferred to the cold room at 4° C.) for 4 h with periodic agitation (one per hour) to facilitate its saturation with air oxygen. The cells were collected by (3,200 g×15 min, 4° C.)-centrifugation, transferred to the 50 mL Falcon tube, and stored at −80° C. until needed. Typically, ˜3.4 g of wet cell pellet was collected from the 0.45 L culture.

Protein Purification

In preferred embodiment, buffer solutions used for the protein purification contained an EDTA-free protease inhibitor cocktail (Roche, Germany) at concentrations according to the manufacturer recommendations. All protein purifications steps were performed at 4° C. A portion of the frozen cells was thawed on ice for ˜2 hrs, and re-suspended with 30 mL of 50 mM HEPES-NaOH (pH 7.5) buffer, containing 0.3 M NaCl and 5 mM mercaptoethanol (ME) (the HS300 buffer). The cells were disrupted by sonication while keeping the cell suspension in the ice-water mix. To obtain the soluble fraction, disrupted cells were centrifuged at 30,000 g for 1 h. The supernatant (31 mL) was loaded onto 5 mL Talon Metal Affinity resin (Clontech) packed into the plastic gravity column and equilibrated with the binding buffer (HS300+5 mM ME). The flow-through fraction containing the unbound species was collected for further SDS-PAGE analysis, and the resin was washed with 40 mL HS300+5 mM ME buffer. Next, the resin was washed with ˜20 mL HS300+0.5 M NaCl+5 mM ME, and then with ˜30 mL HS300+10 mM imidazole-HCl (pH 7.5). No fluorescent protein species were detected in the washes. Finally, the bound proteins were eluted with HS300+200 mM imidazole-HCl (pH 7.5). The eluate was fractionated into five fractions—Fr. 1 (the first 0.45 mL eluate), Fr. 2 (further 3.1 mL of the eluate), Fr. 3 (2.1 mL), Fr. 4 (1.6 mL) and Fr. 5 (the last 1.8 mL of the eluate). The eluted fractions were supplemented with 1 mM EDTA (to inhibit metal-dependent proteases), and analyzed by PAGE/SDS. The less contaminated fractions (Fr. 1 through Fr.3 containing about 27 mG TqLPNGh) were combined and used for further TqLPNGh purification. The combined TqLPNGh solution was exchanged into HS300 buffer containing 0.1 mM EDTA and 0.25 mM DTT (HS300eD) using two 10 mL Econo-Pac 10 DG columns (Bio-Rad) equilibrated in HS300eD. The exchanged TqLPNGh (˜7 mL) was precipitated by mixing with 10 mL saturated ammonium sulfate (AS) till 2.35 M AS (as final concentration). The AS suspension were equally distributed among 151.5 mL-Eppendorf tubes, and the protein was collected by (21,000 g×15 min, 4° C.)-centrifugation. The protein precipitate was re-dissolved in ˜15 mL 50 mM Tris-HCl (pH 7.5)/0.6 M AS/0.1 mM EDTA/0.5 mM DTT (i.e., ˜1 mL per each tube), clarified by (21,000 g×10 min, 4° C.)-centrifugation, and applied for further purification through hydrophobic interaction chromatography (HIC) on 5 mL HiTrap HP Butyl column (GE Healthcare Life Sciences). To avoid overloading, about ⅓ of the clarified TqLPNGh solution (corresponding to 8-9 mG TqLPNGh) was loaded onto the column equilibrated with the 50 mM Tris-HCl (pH 7.5)/0.7 M AS/0.1 mM EDTA/0.5 mM DTT buffer (the binding buffer). Following the column wash with ˜25 mL binding buffer, the bound protein species were eluted with linear gradient of the ammonium-free buffer containing 0.3 M NaCl, i.e., with 50 mM Tris-HCl (pH 7.5)/0.3 M NaCl/0.1 mM EDTA/0.5 mM DTT using 1 mL/min flow rate. The eluate was fractionated in 2.5 mL portions, and analyzed through absorbance spectrum measurements and SDS-PAGE.

The less contaminated fractions from the three HIC runs were combined (˜40 mL), and mixed with 5.7 mL 50 mM HEPES (pH 7.3)/80% (v/v) glycerol/1 mM EDTA to get 10% (v/v) glycerol as final concentration prior to concentration TqLPNGh solution on 30 kDa cutoff membrane (i.e., on Ultra 15 centrifugal unit, 30 kDa cutoff membrane, Millipore). The TqLPNGh solution was exhaustively exchanged into the 50 mM HEPES (pH 7.3)/0.6 M NaCl/10% (v/v) glycerol/0.1 mM EDTA/0.5 mM TCEP/1 mM DTT on the centrifugal unit by using multiple concentration-dilution steps. ˜1.9 mL exchanged/concentrated TqLPNGh solution was mixed with 2.53 mM 50 mM HEPES (pH 7.3)/1 mM EDTA/80% (v/v) glycerol to get 50% (v/v) glycerol at the final concentration of glycerol for further storage of the TqLPNGh at −20° C. The final buffer composition for TqLPNGh storage was 50 mM HEPES (pH 7.3)/0.3 M NaCl/50% (v/v) glycerol/˜0.6 mM EDTA/0.21 mM TCEP/0.42 mM DTT. The final storage concentration of TqLPNGh (as judged from absorbance spectrum of its diluted solution) was 44.6 μM.

The partial purification of the hTqLPNG on Talon Metal Affinity resin was performed as described for the TqLPNGh construct and as generally described above.

Individual fluorescent proteins, mTurquoise2 and mNeonGreen, and LuxP-NGh fusion were expressed in E. coli BL21 (DE 3) (luxS⁻) transformed with appropriate plasmids (pET21-Tqh, pET21-NGh or pET21-LPNGh, respectively). Preparation of cells for protein purification was performed in the same way as described above for the purification of the TqLPNG biosensor. Due to the high level of the protein expression for each protein (which were 200-250 mG/L of the cell culture), purification included affinity chromatography on Talon resin (which was very similar as that described for TqLPNGh purification) and size exclusion chromatography on Superdex 200 (GE Healthcare Life Sciences).

General Spectroscopic Measurements

A Cary 300 spectrophotometer (Agilent/Varian Technologies) and FluoroMax fluorescence spectrophotometer (Horiba Scientific) equipped with Peltier-based temperature controlled cell holders were used for absorbance and fluorescence measurements, respectively. To characterize fluorescent proteins, all measurements (including TqLPNGh titration with BAI-2) were performed at 20° C. with proteins prepared in 50 mM HEPES buffer (pH 7.5)/0.1 mM EDTA, containing different NaCl concentrations. Fluorescence emission spectra were recorded in a 4 mm fluorescence cell by using 2 nm slit width at both excitation and emission monochromators. Typically, 50 nM protein concentrations were employed for fluorescence measurements. The protein stock solutions were prepared in 50 mM HEPES buffer (pH 7.5)/0.1 mM EDTA/0.3 M NaCl at 2 μM (for the TqLPNG) or 10 μM (for Tqh or NGh) concentration. The protein concentrations were determined based on absorption measurements, and the published values of the molar extinction coefficients—ε_{434 nm}=30,000 M⁻¹ cm⁻¹ and ε_{505 nm}=116,000 M⁻¹ cm⁻¹ for mTurquoise2 (Goedhart et al., 2012) and mNeonGreen Shaner et al., 2013), respectively. Due to negligible absorption of mTurquoise2 at 505 nm, the protein concentration for TqLPNGh biosensor was determined by using molar extinction of mNeonGreen.

Forster Distances and Evaluation for the FRET Efficiency in TqLPNG

The Förster distance (R₀)—is the important characteristic of the donor-acceptor FRET pair which can be phenomenologically determined as the donor-to-acceptor distance (R) at which 50% excited donor molecules are deactivated through the FRET mechanism, so that FRET efficiency (E) equals 0.5 (Van der Meer et al., 1994). Thus, the Förster distance determines sensitivity of donor-acceptor resonance coupling:

E=R₀⁶/(R₀⁶+R⁶)

The Förster distance can be determined as:

R₀=0.211×(κ2×n−4×Q_{D}×J_{DA})^{1/6}(in Å)

where κ²—is the orientation factor between the donor emission transition dipole moment and the acceptor absorbance transition dipole moment; n—is the refractive index (taken from the tabulated data available for water solutions of NaCl); Q_{D}—is the mTurquoise2-donor quantum yield [taken as 0.93 from the reported data—Merola et al. (2014)]; and J_{DA}—is the overlap integral between the donor fluorescence emission F_{D}(λ) spectrum and the acceptor absorption ε_{A}(λ) spectrum (in units of molar extinction, M⁻¹ cm⁻¹): J_{DA}=Σ F_{D}(λ)ε_{A}(λ)λ⁴Δλ/Σ F_{D}(λ)Δλ.

To calculate R₀-values for Tq-NG donor-acceptor pair corresponding to different salt conditions, Tq-donor fluorescence emission (corrected to the spectral sensitivity of the emission detector) and NG-acceptor absorption spectra were measured in the 50 mM HEPES (pH 7.5)/0.1 mM EDTA buffer containing different concentrations of NaCl (0, 0.15, 0.30 and 0.60 M). Although both the donor emission and the acceptor absorption featured a small red shift [which did not exceed 1 nm in the case of the donor emission spectra, and was about 2 nm in the case of the acceptor absorption spectra] when increasing the salt concentration, both the donor quantum yield, the overlap integral and R0-values were practically unaffected by NaCl (see Table 3).

FRET efficiency in TqLPNG was determined under variety of solution conditions by measurements of the emission spectra of TqLPNG (DA-species) alternatively excited at 440 nm and 505 nm. The first wavelength, 440 nm, corresponds to the excitation of the Tq-donor, and, therefore, to the conditions for FRET to occur. The second wavelength, 505 nm, is the wavelength for the selective excitation of NG-acceptor (at which no excitation of the donor happens due to the absence of the donor absorption at 500 nm or larger). Emission spectrum of acceptor at 505 nm, A-DA (λ,λ_{A}), is required for determination the acceptor emission directly excited at 440 nm, dirA-DA (λ, λ_{D}), which is in turn necessary for the determination of FRET efficiency (E_{FRET} or simply, E). The dirA-DA (λ, λ_{D}) spectrum can be easily determined from A-DA (λ,λ_{A}) by taking into account the ratio for the excitation light intensity at 440 and 505 nm, I_{EX}(λ_{D})/I_{EX}(λ_{A}), and the absorption ratio of the acceptor, ε_{A}(λ_{D})/ε_{A}(λ_{A}):

dirA-DA(λ,λ_{D})=[I_{EX}(λ_{D})/I_{EX}(λ_{A})]×[ε_{A}(λ_{D})/ε_{A}(λ_{A})]×A-DA(λ,λ_{A}),

where the excitation light intensity ratio, I_{EX}(λ_{D})/I_{EX}(λ_{A}), is determined through the reading of the reference detector (corrected for the spectral sensitivity); and the acceptor absorption ratio, ε_{A}(λ_{D})/ε_{A}(λ_{A}), is determined from the corrected excitation spectrum of A-species, which is NG-acceptor prepared under corresponding buffer conditions.

In general, emission spectrum of DA at the donor excitation, DA(λ, λ_{D}) [or simply DA(λ)] consists of the donor emission component, DC-DA(λ), and the acceptor emission component, AC-DA(λ):

DA(λ)=DC-DA(λ)+AC-DA(λ)

Both the donor and acceptor emission are affected by FRET—the donor emission in DA is quenched as compared with the donor emission in the absence of FRET [i.e., in the absence of the acceptor], while the acceptor emission is enhanced [as compared with the directly excited acceptor emission, which is the acceptor emission in the absence of FRET, in the absence of the donor] due to extra excitation from FRET:

DC-DA(λ)=k I_{EX}(λ_{D})ε_{D}c_{DA}(1−E)Q_{D}F_{D}(λ)

AC-DA(λ)=dirA(λ)+A_{FRET}(λ)=k I_{EX}(λ_{D}) ε_{A} c_{DA} Q_{A} F_{A}(λ)+k ε_{D} E c_{DA} Q_{A} F_{A}(λ), where k—is the geometric factor [which determines the fraction of the total emission detected]; ε_{D}(λ_{EX}) and ε_{A}(λ_{EX})—are molar extinction of the donor and the acceptor, respectively; c_{DA}—is the biosensor molar concentration; E—is the FRET efficiency; Q_{D} and Q_{A}—are the emission efficiency (aka quantum yield) of the donor and the acceptor, respectively; F_{D}(λ) and F_{A}(λ)—are the spectra [i.e., distribution of the emitted photons over emission wavelengths] of the donor and the acceptor, respectively.

The FRET efficiency can be determined as:

E=[AC-DA(λ)/dirA(λ)−1]/(ε_{D}/ε_{A})

The acceptor emission component can be easily determined through subtraction of the donor emission component from the TqLPNG emission spectrum: AC-DA(λ)=DA(λ)−DC-DA(λ). The donor emission component is in turn determined from the emission spectrum donor in D-species, Tq-D(λ) [the so-called D_{ONLY}-species], normalized to the emission intensity of the donor in DA:

DC-DA(λ)=F_{NORM}×D-D(λ),

where normalization factor is calculated as an average of the constant level for DA(λ)/D(λ) corresponding to the initial spectral range of the donor emission (i.e., 450-480 nm), which does not have acceptor emission contribution. Thus, in the case of TqLPNG, deconvolution of its emission spectrum into the donor and the acceptor emission components is evident.

The donor-to-acceptor absorption ratio, ε_{D}/ε_{A}, at the donor excitation (i.e., at 440 nm) was calculated from the corrected excitation spectra of the donor and the acceptor (recorded for the Tq-D or NG-A species under appropriate buffer conditions), which were normalized at their excitation maximums to the values the their molar extinctions (30,000 and 116,000 M⁻¹ cm⁻¹, respectively). The validity of using excitation spectra for the determination of the donor-to-acceptor absorption ratio, ε_{D}/ε_{A}, as well as for the determination of the acceptor absorption ratio, ε_{A}(λ_{D})/ε_{A}(λ_{A}), was proved by practical identity of the normalized excitation and absorption spectra for either Tq-D or NG-A species.

TqLPNG Biosensor Response for BAI-2 Binding

For measurements associated with examination of ligand-induced response of TqLPNG biosensor fluorescence emission 0.1 mM BAI-2 stock solution was freshly prepared and contained 1 mM DPD and 4 mM boric acid in 50 mM HEPES buffer (pH 7.5) (Semmelhack et al., 2005). To test the biosensor fluorescence response for BAI-2 binding 400 μL samples having the same biosensor concentration (50 nM) and variable BAI-2 concentrations in 50 mM HEPES (pH 7.5)/0.4 mM boric acid/0.1 mM EDTA/0.15 M NaCl were prepared from the protein and the ligand stock solutions using analytical balances. Fluorescence emission spectra were taken following 3 min temperature equilibration of the sample within the temperature controlled cell holder (set for 20° C.). Control experiments for photo-bleaching showed no changes in the fluorescence spectrum during three consecutive spectrum records. Control experiments for the Linker sequences were added to the corresponding domain termini, which were then fixed using the ModLoop server (Fiser et al., 2000; Fiser and Sali, 2003) to combine as a single protein construct. Computational estimates of the binding affinities at each interface, as well as quantification of the number of polar vs. nonpolar contacts at each interface, was done using the PRODIGY web server (Vangone and Bonvin 2015; Xue et al., 2016).

To generate a model of ligand-bound TqLPNG monomer, structural overlaps were done between ligand-free LuxP with the BAI-2-bound crystal structure of LuxP (PDB ID 1JX6) (Chen et al., 2002). In particular, the C-terminal lobes from both LuxP structures were structurally overlapped to get the position and orientation of mTurquoise2 in the ligand-bound model, and similarly a structural overlap of the N-terminal lobes from both LuxP structures gave the location of mNeonGreen. Domain termini and linker sequences were again fixed using ModLoop, followed by binding affinity estimates and interface contact analysis using PRODIGY.

Protein-Protein Docking for the LuxP Homodimer

Docking of two ligand-free LuxP molecules (PDB 1ZHH) or two ligand-bound LuxP molecules (PDB 1JX6) was performed using the same protocol as above with HADDOCK2.2. Binding affinity estimates and interface contact analysis of the top predicted poses were then performed using PRODIGY.

### Tables

TABLE 1
Pathogenic organisms that use BAI-2 for QS

| BACTERIA | HOST/DISEASE |
|---|---|
| Vibrio harveyi | Fish and aquatic animals |
| Vibrio parahaemolyticus | (for example, Early Mortality Syndrome) |
| Vibrio cholerae | Humans (cholera) |
| Salmonella ser. typhimurium | Humans and Animals (food poisoning) |
| E. coli H0157:H7 | Humans (food poisoning) |
| Helicobacter pylori | Humans (ulcer) |
| Streptococcus mutans | Humans (infections) |

TABLE 2
Bacterial strains and plasmids

| STRAINS | GENOTYPE | ORIGIN |
|---|---|---|
| BL21 DE3 (luxS) | ΔluxS | Chen et al. |
| | | (2002) |
| BL21-TqLPNGh | BL21 DE3 (luxS) transformed | This invention |
| | with pET21-TqLPNGh | |
| BL21-Tqh | BL21 DE3 (luxS) transformed | This invention |
| | with pET21-Tqh | |
| BL21-NGh | BL21 DE3 (luxS) transformed | This invention |
| | with pET21-NGh | |
| PLASMID | DESCRIPTION | ORIGIN |
| pET21a(+) | Low copy plasmid vector for | Novagen |
| | expression of proteins under the | |
| | control of T7 promoter. Ap^{R} | |
| pET21-TqLPNGh | Plasmid for expression of bio | This invention |
| | sensor protein | |
| pET21-Tqh | Plasmid for expression of donor | This invention |
| | fluorescent protein | |
| pET21-NGh | Plasmid for expression of | This invention |
| | acceptor fluorescent protein | |

TABLE 3
Förster distances for mTurquoise2-mNeonGreen donor-acceptor
pair at different salt concentrations

| | | Overlap integral, | Forster |
|---|---|---|---|
| Buffer | Refractive | J_{DA} (M⁻¹ cm⁻¹ | distance, R₀ |
| composition | index | nm⁴ units) | (Å) |
| no salt | 1.3330 | 2.9386 x 10¹⁵ | 60.88 |
| 0.15M NaCl | 1.3345 | 2.9750 × 10¹⁵ | 60.96 |
| 0.30M NaCl | 1.3360 | 2.9790 × 10¹⁵ | 60.93 |
| 0.60M NaCl | 1.3390 | 2.9879 × 10¹⁵ | 60.87 |

The Förster distance for Tq-NG donor-acceptor pair were measured in 50 mM HEPES (pH 7.5)/0.1 mM EDTA buffer containing different concentration of NaCl (at 20° C.). mTurquoise2 quantum yield was taken as 0.93 [Merola et al. (2014)]. The value of the orientation factor was taken as 2/3.

### REFERENCES

[1] Chen, X., S. Schauder, N. Potier, A. Van Dorsselaer, I. Pelczer, B. L. Bassler & F. M. Hughson, (2002) Structural identification of a bacterial quorum-sensing signal containing boron. Nature 415: 545-549.
[2] Defoirdt, T., (2017) Quorum-Sensing Systems as Targets for Antivirulence Therapy. Trends Microbiol.
[3] Goedhart, J., D. von Stetten, M. Noirclerc-Savoye, M. Lelimousin, L. Joosen, M. A. Hink, L. van Weeren, T. W. Gadella, Jr. & A. Royant, (2012) Structure-guided evolution of cyan fluorescent proteins towards a quantum yield of 93%. Nat Commun 3: 751.
[4] Maniatis, T., E. F. Fritsch & J. Sambrook, (1982) Molecular cloning: a laboratory manual. Cold spring harbor laboratory Cold Spring Harbor, N.Y.
[5] Merola, F., Fredj, A., Betolngar, D.-B., Ziegler C., Erard, M. & Pasquier H. (2014) Newly engineered cyan fluorescence proteins with enhanced performances for live cell FRET imaging. Biotechnol. J. 9: 180-191.
[6] Neiditch, M. B., M. J. Federle, S. T. Miller, B. L. Bassler & F. M. Hughson, (2005) Regulation of LuxPQ receptor activity by the quorum-sensing signal autoinducer-2. Mol Cell 18: 507-518.
[7] Pettersen, E. F., T. D. Goddard, C. C. Huang, G. S. Couch, D. M. Greenblatt, E. C. Meng & T. E. Ferrin, (2004) UCSF Chimera—a visualization system for exploratory research and analysis. J Comput Chem 25: 1605-1612.
[8] Rajamani, S., J. Zhu, D. Pei & R. Sayre, (2007) A LuxP-FRET-Based Reporter for the Detection and Quantification of AI-2 Bacterial Quorum-Sensing Signal Compounds. Biochemistry 46: 3990-3997.
[9] Raut, N., S. Joel, P. Pasini & S. Daunert, (2015) Bacterial autoinducer-2 detection via an engineered quorum sensing protein. Anal Chem 87: 2608-2614.
[10] Semmelhack, M. F., S. R. Campagna, M. J. Federle & B. L. Bassler, (2005) An expeditious synthesis of DPD and boron binding studies. Org Lett 7: 569-572.
[11] Shaner, N. C., G. G. Lambert, A. Chammas, Y. Ni, P. J. Cranfill, M. A. Baird, B. R. Sell, J. R. Allen, R. N. Day, M. Israelsson, M. W. Davidson & J. Wang, (2013) A bright monomeric green fluorescent protein derived from Branchiostoma lanceolatum. Nat Methods 10: 407-409.
[12] Thompson, J. A., R. A. Oliveira, A. Djukovic, C. Ubeda & K. B. Xavier, (2015) Manipulation of the quorum sensing signal AI-2 affects the antibiotic-treated gut microbiota. Cell Rep 10: 1861-1871.
[13] Van Der Meer, B. W., G. Coker & S.-Y. S. Chen, (1994) Resonance energy transfer: theory and data. Wiley-VCH.
[14] Whiteley, M., S. P. Diggle & E. P. Greenberg, (2017) Progress in and promise of bacterial quorum sensing research. Nature 551: 313-320.
[15] Zhu, J. & D. Pei, (2008) A LuxP-Based Fluorescent Sensor for Bacterial Autoinducer II. ACS Chem Biol 3: 110-119.
[16] U.S. patent application Ser. No. 11/789,479
[17] U.S. Pat. No. 5,998,204 (expired)
[18] U.S. Pat. No. 6,469,154
[19] Chen, X., S. Schauder, N. Potier, A. Van Dorsselaer, I. Pelczer, B. L. Bassler & F. M. Hughson, (2002) Structural identification of a bacterial quorum-sensing signal containing boron. Nature 415: 545-549.
[20] Clavel, D., G. Gotthard, D. von Stetten, D. De Sanctis, H. Pasquier, G. G. Lambert, N. C. Shaner & A. Royant, (2016) Structural analysis of the bright monomeric yellow-green fluorescent protein mNeonGreen obtained by directed evolution. Acta Crystallogr D Struct Biol 72: 1298-1307.
[21] de Vries S. J. & A. M. J. J. Bonvin, (2011) CPORT: a consensus interface predictor and its performance in predictoin-driven docking with HADDOCK. PLoS One 6: e17695. Dominguez, C., R. Boelens & A. M. J. J. Bonvin, (2003) HADDOCK: a protein-protein docking approach based on biochemical and/or biophysical information. J Am Chem Soc 125: 1731-1737.
[22] Fiser, A., R. K. Do & A. Sali, (2000) Modeling of loops in protein structures. Protein Sci 9: 1753-1773.
[23] Fiser, A. & A. Sali, (2003) ModLoop: automated modeling of loops in protein structures. Bioinformatics 19: 2500-2501.

SEQUENCE IDENTIFICATION
DNA
TqLPNGh biosensor protein
Artificial
SEQ ID NO: 1
CATATGGTATCAAAGGGTGAAGAACTTTTCACCGGTGTTGTGCCAATTTTGGTCGAGTTAGACGGTGATG
TGAATGGCCACAAATTCTCTGTCTCCGGCGAAGGTGAGGGTGATGCAACCTATGGCAAACTGACGCTGAA
ATTTATTTGCACCACCGGGAAGCTTCCCGTTCCTTGGCCTACTTTGGTTACCACCTTGTCTTGGGGTGTG
CAGTGCTTCGCACGTTATCCTGACCACATGAAACAGCACGATTTTTTTAAAAGTGCAATGCCGGAAGGCT
ATGTCCAGGAACGTACTATTTTTTTTAAGGATGATGGCAACTATAAAACCCGTGCCGAGGTCAAATTTGA
AGGCGACACCTTGGTTAACCGAATTGAACTGAAAGGTATTGATTTTAAAGAAGATGGTAATATTCTGGGT
CACAAATTGGAGTATAACTATTTCAGCGACAACGTTTATATTACCGCAGATAAACAAAAAAACGGTATCA
AAGCCAACTTTAAAATTAGACACAATATCGAAGATGGCGGTGTCCAATTAGCCGACCACTATCAGCAGAA
CACGCCGATCGGTGATGGTCCTGTGCTGCTGCCGGATAATCATTACCTTTCAACACAGAGCAAACTGAGC
AAAGACCCGAACGAGAAACGTGACCACATGGTACTGCTGGAATTTGTGACAGCTGCGGGCATTACGCTTG
GGATGGACGAGCTATATAAAACGGCCGGGGGTGTGCTTAATGGCTATTGGGGCTATCAGGAGTTTCTGGA
TGAGTTCCCAGAACAGCGCAATCTTACGAACGCCCTGTCTGAAGCGGTTCGGGCGCAGCCTGTGCCTCTG
TCTAAGCCGACTCAACGTCCTATCAAGATTTCGGTCGTATACCCTGGGCAGCAAGTTTCCGATTACTGGG
TGCGTAACATCGCAAGCTTTGAAAAACGTCTGTATAAACTGAACATTAATTACCAACTGAACCAAGTGTT
CACCCGACCCAATGCCGATATTAAACAACAGTCACTCAGTCTGATGGAGGCCCTGAAATCCAAATCGGAC
TACCTGATCTTTACCTTGGATACGACTCGCCACCGTAAATTTGTGGAGCACGTACTCGATAGTACAAATA
CGAAACTGATTTTACAGAATATAACGACGCCGGTACGGGAATGGGACAAACACCAGCCGTTCCTGTATGT
GGGTTTTGACCATGCTGAAGGTTCCCGAGAACTGGCAACCGAATTCGGTAAATTTTTCCCGAAACATACG
TATTACTCTGTGCTCTATTTCTCGGAAGGCTACATCAGCGATGTACGTGGAGATACCTTTATCCATCAGG
TGAATCGGGATAATAACTTTGAGCTTCAGTCCGCTTACTACACCAAGGCGACCAAGCAATCCGGTTATGA
TGCTGCCAAGGCGAGCTTGGCAAAGCACCCTGACGTTGACTTTATTTATGCCTGCTCGACGGACGTAGCT
CTGGGTGCAGTAGATGCACTGGCAGAGCTCGGGAGAGAGGATATTATGATCAACGGTTGGGGAGGGGGGT
CCGCGGAATTAGACGCGATACAGAAAGGTGATCTGGATATTACCGTCATGCGTATGAACGATGACACGGG
TATTGCCATGGCAGAAGCGATTAAATGGGATCTGGAAGACAAACCAGTACCGACCGTATATAGTGGCGAC
TTTGAAATCGTAACCAAAGCGGATTCCCCGGAACGAATCGAAGCGCTGAAGAAGCGTGCTTTTCGCTATT
CCGATAACGGTGGGGCGGCTGCCATGGCAAGCCTTCCGGCTACCCATGAGCTTCATATTTTCGGCAGTAT
TAATGGCGTAGACTTTGACATGGTGGGTCAGGGAACCGGAAATCCCAATGATGGCTATGAAGAGTTGAAC
CTGAAATCTACCAAGGGCGACCTCCAGTTTTCACCTTGGATTTTGGTGCCACACATCGGTTATGGATTTC
ATCAGTATTTACCTTATCCGGATGGAATGAGTCCGTTCCAGGCGGCCATGGTTGATGGTTCCGGTTATCA
GGTCCATCGCACGATGCAGTTCGAGGACGGGGCCTCTCTGACGGTGAATTATCGTTATACCTATGAAGGT
TCACATATAAAAGGTGAAGCTCAGGTAAAAGGCACGGGTTTTCCGGCGGATGGCCCGGTGATGACCAACT
CCCTCACGGCGGCGGACTGGTGCCGGAGCAAAAAGACGTATCCGAATGATAAAACCATCATTAGCACCTT
TAAATGGTCATACACGACTGGCAACGGTAAACGCTACAGATCCACCGCTCGCACCACATATACTTTTGCT
AAACCTATGGCCGCAAACTATTTGAAAAACCAGCCTATGTACGTTTTCCGTAAAACCGAACTGAAGCACA
GTAAAACTGAACTGAATTTCAAAGAATGGCAAAAAGCATTTACGGATGTTATGGGTATGGATGAGCTGTA
TAAGCATCACCATCATCATCATTAATAGCTCGAG
Amino Acid
TqLPNGh biosensor protein
Artificial
SEQ ID NO: 2
MVSKGEELFTGVVPILVELDGDVNGHKFSVSGEGEGDATYGKLTLKFICTTGKLPVPWPTLVTTLSWGVQ
CFARYPDHMKQHDFFKSAMPEGYVQERTIFFKDDGNYKTRAEVKFEGDTLVNRIELKGIDFKEDGNILGH
KLEYNYFSDNVYITADKQKNGIKANFKIRHNIEDGGVQLADHYQQNTPIGDGPVLLPDNHYLSTQSKLSK
DPNEKRDHMVLLEFVTAAGITLGMDELYKTAGGVLNGYWGYQEFLDEFPEQRNLTNALSEAVRAQPVPLS
KPTQRPIKISVVYPGQQVSDYWVRNIASFEKRLYKLNINYQLNQVFTRPNADIKQQSLSLMEALKSKSDY
LIFTLDTTRHRKFVEHVLDSTNTKLILQNITTPVREWDKHQPFLYVGFDHAEGSRELATEFGKFFPKHTY
YSVLYFSEGYISDVRGDTFIHQVNRDNNFELQSAYYTKATKQSGYDAAKASLAKHPDVDFIYACSTDVAL
GAVDALAELGREDIMINGWGGGSAELDAIQKGDLDITVMRMNDDTGIAMAEAIKWDLEDKPVPTVYSGDF
EIVTKADSPERIEALKKRAFRYSDNGGAAAMASLPATHELHIFGSINGVDFDMVGQGTGNPNDGYEELNL
KSTKGDLQFSPWILVPHIGYGFHQYLPYPDGMSPFQAAMVDGSGYQVHRTMQFEDGASLTVNYRYTYEGS
HIKGEAQVKGTGFPADGPVMTNSLTAADWCRSKKTYPNDKTIISTFKWSYTTGNGKRYRSTARTTYTFAK
PMAANYLKNQPMYVFRKTELKHSKTELNFKEWQKAFTDVMGMDELYKHHHHHH
Amino Acid
TqLPNGh biosensor protein lacking H6 sequence at C-terminal end
Artificial
SEQ ID NO: 3
MVSKGEELFTGVVPILVELDGDVNGHKFSVSGEGEGDATYGKLTLKFICTTGKLPVPWPTLVTTLSWGVQ
CFARYPDHMKQHDFFKSAMPEGYVQERTIFFKDDGNYKTRAEVKFEGDTLVNRIELKGIDFKEDGNILGH
KLEYNYFSDNVYITADKQKNGIKANFKIRHNIEDGGVQLADHYQQNTPIGDGPVLLPDNHYLSTQSKLSK
DPNEKRDHMVLLEFVTAAGITLGMDELYKTAGGVLNGYWGYQEFLDEFPEQRNLTNALSEAVRAQPVPLS
KPTQRPIKISVVYPGQQVSDYWVRNIASFEKRLYKLNINYQLNQVFTRPNADIKQQSLSLMEALKSKSDY
LIFTLDTTRHRKFVEHVLDSTNTKLILQNITTPVREWDKHQPFLYVGFDHAEGSRELATEFGKFFPKHTY
YSVLYFSEGYISDVRGDTFIHQVNRDNNFELQSAYYTKATKQSGYDAAKASLAKHPDVDFIYACSTDVAL
GAVDALAELGREDIMINGWGGGSAELDAIQKGDLDITVMRMNDDTGIAMAEAIKWDLEDKPVPTVYSGDF
EIVTKADSPERIEALKKRAFRYSDNGGAAAMASLPATHELHIFGSINGVDFDMVGQGTGNPNDGYEELNL
KSTKGDLQFSPWILVPHIGYGFHQYLPYPDGMSPFQAAMVDGSGYQVHRTMQFEDGASLTVNYRYTYEGS
HIKGEAQVKGTGFPADGPVMTNSLTAADWCRSKKTYPNDKTIISTFKWSYTTGNGKRYRSTARTTYTFAK
PMAANYLKNQPMYVFRKTELKHSKTELNFKEWQKAFTDVMGMDELYK
DNA
Δ24-365 LuxP
Vibrio harveyi
SEQ ID NO: 4
ATGGTATCAAAGGGTGTGCTTAATGGCTATTGGGGCTATCAGGAGTTTCTGGATGAGTTCCCAGAACAGC
GCAATCTTACGAACGCCCTGTCTGAAGCGGTTCGGGCGCAGCCTGTGCCTCTGTCTAAGCCGACTCAACG
TCCTATCAAGATTTCGGTCGTATACCCTGGGCAGCAAGTTTCCGATTACTGGGTGCGTAACATCGCAAGC
TTTGAAAAACGTCTGTATAAACTGAACATTAATTACCAACTGAACCAAGTGTTCACCCGACCCAATGCCG
ATATTAAACAACAGTCACTCAGTCTGATGGAGGCCCTGAAATCCAAATCGGACTACCTGATCTTTACCTT
GGATACGACTCGCCACCGTAAATTTGTGGAGCACGTACTCGATAGTACAAATACGAAACTGATTTTACAG
AATATAACGACGCCGGTACGGGAATGGGACAAACACCAGCCGTTCCTGTATGTGGGTTTTGACCATGCTG
AAGGTTCCCGAGAACTGGCAACCGAATTCGGTAAATTTTTCCCGAAACATACGTATTACTCTGTGCTCTA
TTTCTCGGAAGGCTACATCAGCGATGTACGTGGAGATACCTTTATCCATCAGGTGAATCGGGATAATAAC
TTTGAGCTTCAGTCCGCTTACTACACCAAGGCGACCAAGCAATCCGGTTATGATGCTGCCAAGGCGAGCT
TGGCAAAGCACCCTGACGTTGACTTTATTTATGCCTGCTCGACGGACGTAGCTCTGGGTGCAGTAGATGC
ACTGGCAGAGCTCGGGAGAGAGGATATTATGATCAACGGTTGGGGAGGGGGGTCCGCGGAATTAGACGCG
ATACAGAAAGGTGATCTGGATATTACCGTCATGCGTATGAACGATGACACGGGTATTGCCATGGCAGAAG
CGATTAAATGGGATCTGGAAGACAAACCAGTACCGACCGTATATAGTGGCGACTTTGAAATCGTAACCAA
AGCGGATTCCCCGGAACGAATCGAAGCGCTGAAGAAGCGTGCTTTTCGCTATTCCGATAACGG
Amino Acid
Δ24-365 LuxP
Vibrio harveyi
SEQ ID NO: 5
MVSKGVLNGYWGYQEFLDEFPEQRNLTNALSEAVRAQPVPLSKPTQRPIKISVVYPGQQVSDYWVRNIAS
FEKRLYKLNINYQLNQVFTRPNADIKQQSLSLMEALKSKSDYLIFTLDTTRHRKFVEHVLDSTNTKLILQ
NITTPVREWDKHQPFLYVGFDHAEGSRELATEFGKFFPKHTYYSVLYFSEGYISDVRGDTFIHQVNRDNN
FELQSAYYTKATKQSGYDAAKASLAKHPDVDFIYACSTDVALGAVDALAELGREDIMINGWGGGSAELDA
IQKGDLDITVMRMNDDTGIAMAEAIKWDLEDKPVPTVYSGDFEIVTKADSPERIEALKKRAFRYSDNG
DNA
mNeonGreen-His6 fluorescent protein
Branchiostoma lanceolatum
SEQ ID NO: 6
ATGGCAAGCCTTCCGGCTACCCATGAGCTTCATATTTTCGGCAGTATTAATGGCGTAGACTTTGACATGG
TGGGTCAGGGAACCGGAAATCCCAATGATGGCTATGAAGAGTTGAACCTGAAATCTACCAAGGGCGACCT
CCAGTTTTCACCTTGGATTTTGGTGCCACACATCGGTTATGGATTTCATCAGTATTTACCTTATCCGGAT
GGAATGAGTCCGTTCCAGGCGGCCATGGTTGATGGTTCCGGTTATCAGGTCCATCGCACGATGCAGTTCG
AGGACGGGGCCTCTCTGACGGTGAATTATCGTTATACCTATGAAGGTTCACATATAAAAGGTGAAGCTCA
GGTAAAAGGCACGGGTTTTCCGGCGGATGGCCCGGTGATGACCAACTCCCTCACGGCGGCGGACTGGTGC
CGGAGCAAAAAGACGTATCCGAATGATAAAACCATCATTAGCACCTTTAAATGGTCATACACGACTGGCA
ACGGTAAACGCTACAGATCCACCGCTCGCACCACATATACTTTTGCTAAACCTATGGCCGCAAACTATTT
GAAAAACCAGCCTATGTACGTTTTCCGTAAAACCGAACTGAAGCACAGTAAAACTGAACTGAATTTCAAA
GAATGGCAAAAAGCATTTACGGATGTTATGGGTATGGATGAGCTGTATAAGCATCACCATCATCATCAT
Amino Acid
mNeonGreen-His6 fluorescent protein
Branchiostoma lanceolatum
SEQ ID NO: 7
MVSKGEEDNMASLPATHELHIFGSINGVDFDMVGQGTGNPNDGYEELNLKSTKGDLQFSPWILVPHIGYG
FHQYLPYPDGMSPFQAAMVDGSGYQVHRTMQFEDGASLTVNYRYTYEGSHIKGEAQVKGTGFPADGPVMT
NSLTAADWCRSKKTYPNDKTIISTFKWSYTTGNGKRYRSTARTTYTFAKPMAANYLKNQPMYVFRKTELK
HSKTELNFKEWQKAFTDVMGMDELYKHHHHHH
DNA
mTurquoise-His6 fluorescent protein
Aequorea victoria
SEQ ID NO: 8
ATGGTATCAAAGGGTGAAGAACTTTTCACCGGTGTTGTGCCAATTTTGGTCGAGTTAGACGGTGATGTGA
ATGGCCACAAATTCTCTGTCTCCGGCGAAGGTGAGGGTGATGCAACCTATGGCAAACTGACGCTGAAATT
TATTTGCACCACCGGGAAGCTTCCCGTTCCTTGGCCTACTTTGGTTACCACCTTGTCTTGGGGTGTGCAG
TGCTTCGCACGTTATCCTGACCACATGAAACAGCACGATTTTTTTAAAAGTGCAATGCCGGAAGGCTATG
TCCAGGAACGTACTATTTTTTTTAAGGATGATGGCAACTATAAAACCCGTGCCGAGGTCAAATTTGAAGG
CGACACCTTGGTTAACCGAATTGAACTGAAAGGTATTGATTTTAAAGAAGATGGTAATATTCTGGGTCAC
AAATTGGAGTATAACTATTTCAGCGACAACGTTTATATTACCGCAGATAAACAAAAAAACGGTATCAAAG
CCAACTTTAAAATTAGACACAATATCGAAGAT GGCGGTGTCCAATTAGCCGACCACTATCAGCAGAACAC
GCCGATCGGTGATGGTCCTGTGCTGCTGCCGGATAATCATTACCTTTCAACACAGAGCAAACTGAGCAAA
GACCCGAACGAGAAACGTGACCACATGGTACTGCTGGAATTTGTGACAGCTGCGGGCATTACGCTTGGGA
TGGACGAGCTATATAAACATCACCATCATCATCATTAATAG
Amino Acid
mTurquoise-His6 fluorescent protein
Aequorea victoria
SEQ ID NO: 9
MVSKGEELFTGVVPILVELDGDVNGHKFSVSGEGEGDATYGKLTLKFICTTGKLPVPWPTLVTTLSWGVQ
CFARYPDHMKQHDFFKSAMPEGYVQERTIFFKDDGNYKTRAEVKFEGDTLVNRIELKGIDFKEDGNILGH
KLEYNYFSDNVYITADKQKNGIKANFKIRHNIEDGGVQLADHYQQNTPIGDGPVLLPDNHYLSTQSKLSK
DPNEKRDHMVLLEFVTAAGITLGMDELYKHHHHHH
DNA
Plasmid - pET-21a(+)-TqLPNGh
Artificial
SEQ ID NO: 10
TGGCGAATGGGACGCGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAGCTGATTTA
ACAAAAATTTAACGCGAATTTTAACAAAATATTAACGTTTACAATTTCAGGTGGCACTTTTCGGGGAAAT
GTGCGCGGAACCCCTATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGCCATACCAAACGAC
GAGCGTGACACCACGATGCCTGCAGCAATGGCAACAACGTTGCGCAAACTATTAACTGGCGAACTACTTA
CTCTAGCTTCCCGGCAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCA
AGTTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAACTGGCTTC
AGCAGAGCGCAGATACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCTGATTCTGTGGA
TAACCGTATTACCGCCTTTGAGTGAGCTGATACCGCTCGCCGCAGCCGAACGACCGAGCGCAGCGAGTCA
GTGAGCGAGGAAGCGGAAGAGCGCCTGATGCGGTATTTTCTCCTTACGCATCTGTGCGGTATTTCACACC
GCATATATGGTGCACTCTCAGTACAATCTGCTCTGATGCCGCATAGTTAAGCCAGTATACACTCCGCTAT
CGCTACGTGACTGGGTCATGGCTGCGCCCCGACACCCGCCAACACCCGCTGACGCGCCCTGACGGGCTTG
TCTGCTCCCGGCATCCGCTTACAGACAAGCTGTGACCGTCTCCGGGAGCTGCATGTGTCAGAGGTTTTCA
CCGTCATCACCGAAACGCGCGAGGCAGCTGCGGTAAAGCTCATCAGCGTGGTCGTGAAGCGATTCACAGA
TGTCTGCCTGTTCATCCGCGTCCAGCTCGTTGAGTTTCTCCAGAAGCGTTAATGTCTGGCTTCTGATAAA
GCGGGCCATGTTAAGGGCGGTTTTTTCCTGTTTGGTCACTGATGCCTCCGTGTAAGGGGGATTTCTGTTC
ATGGGGGTAATGATACCGATGAAACGAGAGAGGATGCTCACGATACGGGTTACTGATGATGAACATGCCC
GGTTACTGGAACGTTGTGAGGGTAAACAACTGGCGGTATGGATGCGGCGGGACCAGAGAAAAATCACTCA
GGGTCAATGCCAGCGCTTCGTTAATACAGATGTAGGTGTTCCACAGGGTAGCCAGCAGCATCCTGCGATG
CAGATCCGGAACATAATGGTGCAGGGCGCTGACTTCCGCGTTTCCAGACTTTACGAAACACGGAAACCGA
AGACCATTCATGTTGTTGCTCAGGTCGCAGACGTTTTGCAGCAGCAGTCGCTTCACGTTCGCTCGCGTAT
CGGTGATTCATTCTGCTAACCAGTAAGGCAACCCCGCCAGCCTAGCCGGGTCCTCAACGACAGGAGCACG
ATCATGCGCACCCGTGGGGCCGCCATGCCGGCGATAATGGCCTGCTTCTCGCCGAAACGTTTGGTGGCGG
GACCAGTGACGAAGGCTTGAGCGAGGGCGTGCAAGATTCCGAATACCGCAAGCGACAGGCCGATCATCGT
CGCGCTCCAGCGAAAGCGGTCCTCGCCGAAAATGACCCAGAGCGCTGCCGGCACCTGTCCTACGAGTTGC
ATGATAAAGAAGACAGTCATAAGTGCGGCGACGATAGTCATGCCCCGCGCCCACCGGAAGGAGCTGACTG
GGTTGAAGGCTCTCAAGGGCATCGGTCGAGATCCCGGTGCCTAATGAGTGAGCTAACTTACATTAATTGC
GTTGCGCTCACTGCCCGCTTTCCAGTCGGGAAACCTGTCGTGCCAGCTGCATTAATGAATCGGCCAACGC
GCGGGGAGAGGCGGTTTGCGTATTGGGCGCCAGGGTGGTTTTTCTTTTCACCAGTGAGACGGGCAACAGC
TGATTGCCCTTCACCGCCTGGCCCTGAGAGAGTTGCAGCAAGCGGTCCACGCTGGTTTGCCCCAGCAGGC
GAAAATCCTGTTTGATGGTGGTTAACGGCGGGATATAACATGAGCTGTCTTCGGTATCGTCGTATCCCAC
TACCGAGATATCCGCACCAACGCGCAGCCCGGACTCGGTAATGGCGCGCATTGCGCCCAGCGCCATCTGA
TCGTTGGCAACCAGCATCGCAGTGGGAACGATGCCCTCATCAGCATTTGCATGGTTTGTTGAAAACCGGA
CATGGCACTCCAGTCGCCTTCCCGTTCCGCTATCGGCTGAATTTGATTGCGAGTGAGATATTTATGCCAG
CCAGCCAGACGCAGACGCGCCGAGACAGAACTTAATGGGCCCGCTAACAGCGCGATTTGCTGGTGACCCA
ATGCGACCAGATGCTCCACGCCCAGTCGCGTACCGTCTTCATGGGAGAAAATAATACTGTTGATGGGTTC
TGGTCAGAGACATCAAGAAATAACGCCGGAACATTAGTGCAGGCAGCTTCCACAGCAATGGCATCCTGGT
CATCCAGCGGATAGTTAATGATCAGCCCACTGACGCGTTGCGCGAGAAGATTGTGCACCGCCGCTTTACA
GGCTTCGACGCCGCTTCGTTCTACCATCGACACCACCACGCTGGCACCCAGTTGATCGGCGCGAGATTTA
ATCGCCGCGACAATTTGCGACGGCGCGTGCAGGGCCAGACTGGAGGTGGCAACGCCAATCAGCAACGACT
GTTTGCCCGCCAGTTGTTGTGCCACGCGGTTGGGAATGTAATTCAGCTCCGCCATCGCCGCTTCCACTTT
TTCCCGCGTTTTCGCAGAAACGTGGCTGGCCTGGTTCACCACGCGGGAAACGGTCTGATAAGAGACACCG
GCATACTCTGCGACATCGTATAACGTTACTGGTTTCACATTCACCACCCTGAATTGACTCTCTTCCGGGC
GCTATCATGCCATACCGCGAAAGGTTTTGCGCCATTCGATGGTGTCCGGGATCTCGACGCTCTCCCTTAT
GCGACTCCTGCATTAGGAAGCAGCCCAGTAGTAGGTTGAGGCCGTTGAGCACCGCCGCCGCAAGGAATGG
TGCATGCAAGGAGATGGCGCCCAACAGTCCCCCGGCCACGGGGCCTGCCACCATACCCACGCCGAAACAA
GCGCTCATGAGCCCGAAGTGGCGAGCCCGATCTTCCCCATCGGTGATGTCGGCGATATAGGCGCCAGCAA
CCGCACCTGTGGCGCCGGTGATGCCGGCCACGATGCGTCCGGCGTAGAGGATCGAGATCTCGATCCCGCG
AAATTAATACGACTCACTATAGGGGAATTGTGAGCGGATAACAATTCCCCTCTAGAAATAATTTTGTTTA
ACTTTAAGAAGGAGATATACATATGGTATCAAAGGGTGAAGAACTTTTCACCGGTGTTGTGCCAATTTTG
GTCGAGTTAGACGGTGATGTGAATGGCCACAAATTCTCTGTCTCCGGCGAAGGTGAGGGTGATGCAACCT
ATGGCAAACTGACGCTGAAATTTATTTGCACCACCGGGAAGCTTCCCGTTCCTTGGCCTACTTTGGTTAC
CACCTTGTCTTGGGGTGTGCAGTGCTTCGCACGTTATCCTGACCACATGAAACAGCACGATTTTTTTAAA
AGTGCAATGCCGGAAGGCTATGTCCAGGAACGTACTATTTTTTTTAAGGATGATGGCAACTATAAAACCC
GTGCCGAGGTCAAATTTGAAGGCGACACCTTGGTTAACCGAATTGAACTGAAAGGTATTGATTTTAAAGA
AGATGGTAATATTCTGGGTCACAAATTGGAGTATAACTATTTCAGCGACAACGTTTATATTACCGCAGAT
AAACAAAAAAACGGTATCAAAGCCAACTTTAAAATTAGACACAATATCGAAGATGGCGGTGTCCAATTAG
CCGACCACTATCAGCAGAACACGCCGATCGGTGATGGTCCTGTGCTGCTGCCGGATAATCATTACCTTTC
AACACAGAGCAAACTGAGCAAAGACCCGAACGAGAAACGTGACCACATGGTACTGCTGGAATTTGTGACA
GCTGCGGGCATTACGCTTGGGATGGACGAGCTATATAAAACGGCCGGGGGTGTGCTTAATGGCTATTGGG
GCTATCAGGAGTTTCTGGATGAGTTCCCAGAACAGCGCAATCTTACGAACGCCCTGTCTGAAGCGGTTCG
GGCGCAGCCTGTGCCTCTGTCTAAGCCGACTCAACGTCCTATCAAGATTTCGGTCGTATACCCTGGGCAG
CAAGTTTCCGATTACTGGGTGCGTAACATCGCAAGCTTTGAAAAACGTCTGTATAAACTGAACATTAATT
ACCAACTGAACCAAGTGTTCACCCGACCCAATGCCGATATTAAACAACAGTCACTCAGTCTGATGGAGGC
CCTGAAATCCAAATCGGACTACCTGATCTTTACCTTGGATACGACTCGCCACCGTAAATTTGTGGAGCAC
GTACTCGATAGTACAAATACGAAACTGATTTTACAGAATATAACGACGCCGGTACGGGAATGGGACAAAC
ACCAGCCGTTCCTGTATGTGGGTTTTGACCATGCTGAAGGTTCCCGAGAACTGGCAACCGAATTCGGTAA
ATTTTTCCCGAAACATACGTATTACTCTGTGCTCTATTTCTCGGAAGGCTACATCAGCGATGTACGTGGA
GATACCTTTATCCATCAGGTGAATCGGGATAATAACTTTGAGCTTCAGTCCGCTTACTACACCAAGGCGA
CCAAGCAATCCGGTTATGATGCTGCCAAGGCGAGCTTGGCAAAGCACCCTGACGTTGACTTTATTTATGC
CTGCTCGACGGACGTAGCTCTGGGTGCAGTAGATGCACTGGCAGAGCTCGGGAGAGAGGATATTATGATC
AACGGTTGGGGAGGGGGGTCCGCGGAATTAGACGCGATACAGAAAGGTGATCTGGATATTACCGTCATGC
GTATGAACGATGACACGGGTATTGCCATGGCAGAAGCGATTAAATGGGATCTGGAAGACAAACCAGTACC
GACCGTATATAGTGGCGACTTTGAAATCGTAACCAAAGCGGATTCCCCGGAACGAATCGAAGCGCTGAAG
AAGCGTGCTTTTCGCTATTCCGATAACGGTGGGGCGGCTGCCATGGCAAGCCTTCCGGCTACCCATGAGC
TTCATATTTTCGGCAGTATTAATGGCGTAGACTTTGACATGGTGGGTCAGGGAACCGGAAATCCCAATGA
TGGCTATGAAGAGTTGAACCTGAAATCTACCAAGGGCGACCTCCAGTTTTCACCTTGGATTTTGGTGCCA
CACATCGGTTATGGATTTCATCAGTATTTACCTTATCCGGATGGAATGAGTCCGTTCCAGGCGGCCATGG
TTGATGGTTCCGGTTATCAGGTCCATCGCACGATGCAGTTCGAGGACGGGGCCTCTCTGACGGTGAATTA
TCGTTATACCTATGAAGGTTCACATATAAAAGGTGAAGCTCAGGTAAAAGGCACGGGTTTTCCGGCGGAT
GGCCCGGTGATGACCAACTCCCTCACGGCGGCGGACTGGTGCCGGAGCAAAAAGACGTATCCGAATGATA
AAACCATCATTAGCACCTTTAAATGGTCATACACGACTGGCAACGGTAAACGCTACAGATCCACCGCTCG
CACCACATATACTTTTGCTAAACCTATGGCCGCAAACTATTTGAAAAACCAGCCTATGTACGTTTTCCGT
AAAACCGAACTGAAGCACAGTAAAACTGAACTGAATTTCAAAGAATGGCAAAAAGCATTTACGGATGTTA
TGGGTATGGATGAGCTGTATAAGCATCACCATCATCATCATTAATAGCTCGAGCACCACCACCACCACCA
CTGAGATCCGGCTGCTAACAAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAGGAGGAACTATATCCGGAT
DNA
Plasmid - pET21-hTqLPNG
Artificial
SEQ ID NO. 11
TGGCGAATGGGACGCGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAGCTGATTTA
ACAAAAATTTAACGCGAATTTTAACAAAATATTAACGTTTACAATTTCAGGTGGCACTTTTCGGGGAAAT
GTGCGCGGAACCCCTATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGCCATACCAAACGAC
GAGCGTGACACCACGATGCCTGCAGCAATGGCAACAACGTTGCGCAAACTATTAACTGGCGAACTACTTA
CTCTAGCTTCCCGGCAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCA
AGTTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAACTGGCTTC
AGCAGAGCGCAGATACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCTGATTCTGTGGA
TAACCGTATTACCGCCTTTGAGTGAGCTGATACCGCTCGCCGCAGCCGAACGACCGAGCGCAGCGAGTCA
GTGAGCGAGGAAGCGGAAGAGCGCCTGATGCGGTATTTTCTCCTTACGCATCTGTGCGGTATTTCACACC
GCATATATGGTGCACTCTCAGTACAATCTGCTCTGATGCCGCATAGTTAAGCCAGTATACACTCCGCTAT
CGCTACGTGACTGGGTCATGGCTGCGCCCCGACACCCGCCAACACCCGCTGACGCGCCCTGACGGGCTTG
TCTGCTCCCGGCATCCGCTTACAGACAAGCTGTGACCGTCTCCGGGAGCTGCATGTGTCAGAGGTTTTCA
CCGTCATCACCGAAACGCGCGAGGCAGCTGCGGTAAAGCTCATCAGCGTGGTCGTGAAGCGATTCACAGA
TGTCTGCCTGTTCATCCGCGTCCAGCTCGTTGAGTTTCTCCAGAAGCGTTAATGTCTGGCTTCTGATAAA
GCGGGCCATGTTAAGGGCGGTTTTTTCCTGTTTGGTCACTGATGCCTCCGTGTAAGGGGGATTTCTGTTC
ATGGGGGTAATGATACCGATGAAACGAGAGAGGATGCTCACGATACGGGTTACTGATGATGAACATGCCC
GGTTACTGGAACGTTGTGAGGGTAAACAACTGGCGGTATGGATGCGGCGGGACCAGAGAAAAATCACTCA
GGGTCAATGCCAGCGCTTCGTTAATACAGATGTAGGTGTTCCACAGGGTAGCCAGCAGCATCCTGCGATG
CAGATCCGGAACATAATGGTGCAGGGCGCTGACTTCCGCGTTTCCAGACTTTACGAAACACGGAAACCGA
AGACCATTCATGTTGTTGCTCAGGTCGCAGACGTTTTGCAGCAGCAGTCGCTTCACGTTCGCTCGCGTAT
CGGTGATTCATTCTGCTAACCAGTAAGGCAACCCCGCCAGCCTAGCCGGGTCCTCAACGACAGGAGCACG
ATCATGCGCACCCGTGGGGCCGCCATGCCGGCGATAATGGCCTGCTTCTCGCCGAAACGTTTGGTGGCGG
GACCAGTGACGAAGGCTTGAGCGAGGGCGTGCAAGATTCCGAATACCGCAAGCGACAGGCCGATCATCGT
CGCGCTCCAGCGAAAGCGGTCCTCGCCGAAAATGACCCAGAGCGCTGCCGGCACCTGTCCTACGAGTTGC
ATGATAAAGAAGACAGTCATAAGTGCGGCGACGATAGTCATGCCCCGCGCCCACCGGAAGGAGCTGACTG
GGTTGAAGGCTCTCAAGGGCATCGGTCGAGATCCCGGTGCCTAATGAGTGAGCTAACTTACATTAATTGC
GTTGCGCTCACTGCCCGCTTTCCAGTCGGGAAACCTGTCGTGCCAGCTGCATTAATGAATCGGCCAACGC
GCGGGGAGAGGCGGTTTGCGTATTGGGCGCCAGGGTGGTTTTTCTTTTCACCAGTGAGACGGGCAACAGC
TGATTGCCCTTCACCGCCTGGCCCTGAGAGAGTTGCAGCAAGCGGTCCACGCTGGTTTGCCCCAGCAGGC
GAAAATCCTGTTTGATGGTGGTTAACGGCGGGATATAACATGAGCTGTCTTCGGTATCGTCGTATCCCAC
TACCGAGATATCCGCACCAACGCGCAGCCCGGACTCGGTAATGGCGCGCATTGCGCCCAGCGCCATCTGA
TCGTTGGCAACCAGCATCGCAGTGGGAACGATGCCCTCATTCAGCATTTGCATGGTTTGTTGAAAACCGG
ACATGGCACTCCAGTCGCCTTCCCGTTCCGCTATCGGCTGAATTTGATTGCGAGTGAGATATTTATGCCA
GCCAGCCAGACGCAGACGCGCCGAGACAGAACTTAATGGGCCCGCTAACAGCGCGATTTGCTGGTGACCC
AATGCGACCAGATGCTCCACGCCCAGTCGCGTACCGTCTTCATGGGAGAAAATAATACTGTTGATGGGTG
TCTGGTCAGAGACATCAAGAAATAACGCCGGAACATTAGTGCAGGCAGCTTCCACAGCAATGGCATCCTG
GTCATCCAGCGGATAGTTAATGATCAGCCCACTGACGCGTTGCGCGAGAAGATTGTGCACCGCCGCTTTA
CAGGCTTCGACGCCGCTTCGTTCTACCATCGACACCACCACGCTGGCACCCAGTTGATCGGCGCGAGATT
TAATCGCCGCGACAATTTGCGACGGCGCGTGCAGGGCCAGACTGGAGGTGGCAACGCCAATCAGCAACGA
CTGTTTGCCCGCCAGTTGTTGTGCCACGCGGTTGGGAATGTAATTCAGCTCCGCCATCGCCGCTTCCACT
TTTTCCCGCGTTTTCGCAGAAACGTGGCTGGCCTGGTTCACCACGCGGGAAACGGTCTGATAAGAGACAC
CGGCATACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTCTCCCTT
ATGCGACTCCTGCATTAGGAAGCAGCCCAGTAGTAGGTTGAGGCCGTTGAGCACCGCCGCCGCAAGGAAT
GGTGCATGCAAGGAGATGGCGCCCAACAGTCCCCCGGCCACGGGGCCTGCCACCATACCCACGCCGAAAC
AAGCGCTCATGAGCCCGAAGTGGCGAGCCCGATCTTCCCCATCGGTGATGTCGGCGATATAGGCGCCAGC
AACCGCACCTGTGGCGCCGGTGATGCCGGCCACGATGCGTCCGGCGTAGAGGATCGAGATCTCGATCCCG
CGAAATTAATACGACTCACTATAGGGGAATTGTGAGCGGATAACAATTCCCCTCTAGAAATAATTTTGTT
TAACTTTAAGAAGGAGATATACATATGGGCAGCAGCCATCATCATCATCATCACGGAAGCAGTGGCCGTA
AAGGCGAGGAGCTGTTCACAGGCGTCGTGCCGATCCTGGTAGAACTCGATGGGGATGTCAATGGACATAA
GTTTAGCGTCTCGGGAGAAGGGGAGGGTGACGCCACGTATGGGAAGCTTACGCTGAAATTTATTTGCACC
ACGGGCAAATTACCGGTCCCGTGGCCTACCCTTGTAACCACTCTCTCTTGGGGAGTGCAGTGCTTCGCGA
GGTACCCAGATCACATGAAACAACATGATTTTTTCAAATCGGCGATGCCGGAAGGCTATGTGCAAGAGCG
CACAATCTTTTTTAAAGATGACGGTAATTACAAGACCCGAGCCGAAGTAAAATTTGAGGGAGATACACTG
GTGAACCGTATTGAACTGAAAGGTATTGACTTTAAAGAGGACGGCAATATCCTGGGCCATAAGCTGGAAT
ACAATTATTTTTCTGATAACGTATATATTACGGCCGACAAACAGAAGAACGGTATCAAAGCGAATTTTAA
AATTCGCCACAATATCGAAGACGGGGGTGTTCAGTTGGCCGATCATTATCAACAGAACACTCCGATTGGG
GATGGCCCCGTACTGCTGCCAGATAATCATTACCTCTCTACTCAGTCAAAGCTGAGCAAAGATCCAAACG
AGAAACGTGATCACATGGTTTTACTGGAGTTTGTCACGGCCGCCGGTATTACCCTGGGAATGGATGAACT
GTATAAGACCGCAGGGGGCGTGCTGAACGGCTATTGGGGCTATCAGGAATTCCTGGACGAATTTCCGGAA
CAGCGAAATTTAACCAACGCACTGAGCGAAGCAGTACGCGCCCAACCGGTGCCTCTGTCTAAACCCACCC
AACGCCCGATTAAGATTTCTGTTGTCTACCCGGGGCAGCAGGTGTCTGATTACTGGGTCCGCAACATTGC
CTCCTTTGAGAAGAGGCTGTATAAGCTTAATATCAATTACCAGCTGAACCAAGTGTTCACTCGCCCTAAT
GCGGATATCAAACAGCAATCGCTTTCGCTGATGGAAGCTCTGAAAAGTAAATCTGACTATCTCATCTTTA
CCCTGGACACGACCCGTCACAGAAAGTTCGTCGAACACGTCCTTGATTCAACAAACACAAAACTAATTCT
TCAGAACATCACTACGCCAGTGCGTGAATGGGATAAACATCAGCCGTTTCTTTATGTGGGCTTTGACCAT
GCCGAAGGCAGCCGTGAGTTAGCAACGGAGTTTGGGAAGTTTTTCCCGAAACATACATACTACTCTGTTC
TGTATTTTAGCGAGGGATATATCTCGGACGTGCGCGGTGATACCTTTATTCATCAGGTAAACCGCGATAA
CAACTTTGAACTGCAAAGCGCGTATTATACGAAAGCTACCAAACAAAGCGGCTATGACGCAGCGAAAGCG
TCCCTGGCGAAGCATCCGGATGTCGATTTTATTTACGCATGCAGTACCGACGTAGCATTAGGGGCGGTTG
ACGCACTGGCTGAGTTAGGTCGCGAGGATATTATGATTAATGGTTGGGGTGGGGGTAGTGCCGAATTGGA
CGCTATTCAGAAAGGTGACCTGGATATTACAGTAATGCGTATGAACGATGATACTGGCATTGCCATGGCA
GAGGCAATCAAATGGGATCTGGAAGATAAACCTGTTCCCACGGTATATTCCGGTGATTTTGAAATTGTCA
CTAAAGCAGACAGTCCGGAACGTATAGAAGCACTCAAAAAGCGTGCTTTCCGTTACTCAGATAATGGGGG
CGCTGCCGCCATGGCCTCTCTGCCAGCCACCCATGAACTGCACATTTTTGGTTCGATTAACGGTGTTGAC
TTCGACATGGTAGGGCAGGGTACCGGCAATCCGAATGACGGCTATGAAGAATTGAACCTGAAATCAACGA
AAGGCGATTTGCAGTTTTCTCCGTGGATCCTGGTGCCGCACATTGGCTACGGATTTCACCAGTATCTTCC
GTACCCCGATGGGATGTCACCATTTCAGGCGGCTATGGTGGATGGTTCCGGCTATCAGGTACATCGCACG
ATGCAGTTTGAAGACGGCGCGAGCCTGACGGTTAATTATCGCTACACGTATGAGGGTAGCCACATCAAAG
GCGAAGCGCAAGTAAAAGGTACCGGTTTTCCGGCCGATGGACCGGTTATGACGAACAGTCTAACGGCAGC
CGACTGGTGTCGGTCGAAGAAAACGTATCCGAATGATAAGACCATTATTTCGACGTTCAAGTGGTCCTAT
ACGACCGGAAACGGTAAGCGCTATCGGTCTACCGCACGCACGACATACACTTTCGCTAAACCGATGGCTG
CCAACTACCTTAAAAATCAGCCGATGTATGTATTCCGAAAAACAGAATTAAAACATTCTAAAACCGAACT
TAATTTTAAAGAATGGCAAAAAGCCTTTACAGATGTGATGGGCATGGACGAATTGTACAAATAATAGCTC
GAGCACCACCACCACCACCACTGAGATCCGGCTGCTAACAAAGCCCGAAAGGAAGCTGAGTTGGCTGCTG
CCACCGCTGAGCAATAACTAGCATAACCCCTTGGGGCCTCTAAACGGGTCTTGAGGGGTTTTTTGCTGAA
AGGAGGAACTATATCCGGAT

1. A biosensor for the detection of quorum sensing molecules comprising:
a LuxP binding domain disposed between a Tq protein donor moiety and a NG protein acceptor moiety forming a fusion biosensor protein wherein said LuxP binding domain binds to a LuxP binding domain of a second fusion biosensor protein forming a homodimer fusion biosensor protein;
wherein binding of a quorum sensing molecule to said LuxP binding domain causes dissociation of said homodimer fusion biosensor protein causing enhanced Tq-donor fluorescence emission and simultaneous quenching of NG-acceptor fluorescence emission resulting in a decrease in the Tq-NG acceptor-to-donor fluorescence emission ratio.

2. The biosensor protein of claim 1, wherein said fusion biosensor protein comprises a fusion biosensor protein selected from the group consisting of: amino acid SEQ ID NOs. 2-3.

3. The biosensor protein of claim 1, wherein said wherein said LuxP binding domain comprises amino acid SEQ ID NO. 5.

4. The biosensor protein of claim 3, wherein said wherein said Tq protein donor moiety comprises amino acid SEQ ID NO. 9.

5. The biosensor protein of claim 3, wherein said, wherein said NG protein acceptor moiety comprises amino acid SEQ ID NO. 7.

6. The biosensor protein of claim 4, wherein said Tq protein donor moiety is connected to the N-terminus of said LuxP binding domain, and wherein said NG protein acceptor moiety is connected to the C-terminus of said LuxP binding domain.

7. (canceled)

8. The biosensor protein of claim 1, wherein said quorum sensing molecule comprises BAI-2.

9. The biosensor protein of claim 1, wherein said homodimer fusion biosensor protein comprises a homodimer fusion biosensor protein formed in a low salt environment.

10-14. (canceled)

15. A tripartite fluorescent ratiometric sensor protein comprising:
a ligand binding domain disposed between a protein donor moiety and a protein acceptor moiety forming a fusion biosensor protein wherein said ligand binding domain binds to a ligand binding domain of a second fusion biosensor protein forming a homodimer fusion biosensor protein;
wherein binding of a ligand to said ligand binding domain causes dissociation of said homodimer fusion biosensor protein causing enhanced donor fluorescence emission and simultaneous quenching of acceptor fluorescence emission.

16. The tripartite fluorescent ratiometric sensor protein of claim 15, wherein said ligand binding domain comprises a LuxP binding domain.

17. The tripartite fluorescent ratiometric sensor protein of claim 16, wherein said LuxP binding domain comprises a truncated BAI-2 receptor (LuxP) from Vibrio harveyi.

18. The tripartite fluorescent ratiometric sensor protein of claim 17, wherein said truncated BAI-2 receptor (LuxP) from Vibrio harveyi comprises amino acid SEQ ID NO. 5.

19. The tripartite fluorescent ratiometric sensor protein of claim 15, wherein said donor protein moiety comprises a mTurquoise2 (Tq) monomeric subunit from Aequorea victoria fused to the N-terminus of said ligand binding domain.

20. The tripartite fluorescent ratiometric sensor protein of claim 19, wherein said mTurquoise2 (Tq) monomeric subunit from Aequorea victoria comprises amino acid SEQ ID NO. 9.

21. The tripartite fluorescent ratiometric sensor protein of claim 15, wherein said acceptor protein moiety comprises a mNeonGreen (NG) monomeric subunit from Aequorea victoria fused to the C-terminus of said ligand binding domain.

22. The tripartite fluorescent ratiometric sensor protein of claim 21, wherein said mNeonGreen (NG) monomeric subunit from Aequorea victoria comprises amino acid SEQ ID NO. 7.

23. The tripartite fluorescent ratiometric sensor protein of claim 15, wherein said fusion biosensor protein comprises a fusion biosensor protein selected from the group consisting of: amino acid SEQ ID NOs. 2-3.

24-25. (canceled)

26. The tripartite fluorescent ratiometric sensor protein of claim 15, wherein said homodimer fusion biosensor protein comprises a homodimer fusion biosensor protein formed in a low salt environment.

27. The tripartite fluorescent ratiometric sensor protein of claim 15, wherein said enhanced Tq-donor fluorescence emission and simultaneous quenching of NG-acceptor fluorescence emission comprises a decrease in the Tq-NG acceptor-to-donor fluorescence emission ratio.

28-36. (canceled)

37. A fluorescent biosensor protein selected from the group consisting of: SEQ ID NOs. 2-3.


SEQUENCE LISTING (11 sequences)

SEQ ID NO: 1 - DNA, 2484 bp, Artificial Sequence
  TqLPNGh biosensor protein
catatggtatcaaagggtgaagaacttttcaccggtgttgtgccaattttggtcgagttagacggtgatgtgaatggccacaaattctctgtctccggcgaaggtgagggtgatgcaacctatggcaaactgacgctgaaatttatttgcaccaccgggaagcttcccgttccttggcctactttggttaccaccttgtcttggggtgtgcagtgcttcgcacgttatcctgaccacatgaaacagcacgatttttttaaaagtgcaatgccggaaggctatgtccaggaacgtactattttttttaaggatgatggcaactataaaacccgtgccgaggtcaaatttgaaggcgacaccttggttaaccgaattgaactgaaaggtattgattttaaagaagatggtaatattctgggtcacaaattggagtataactatttcagcgacaacgtttatattaccgcagataaacaaaaaaacggtatcaaagccaactttaaaattagacacaatatcgaagatggcggtgtccaattagccgaccactatcagcagaacacgccgatcggtgatggtcctgtgctgctgccggataatcattacctttcaacacagagcaaactgagcaaagacccgaacgagaaacgtgaccacatggtactgctggaatttgtgacagctgcgggcattacgcttgggatggacgagctatataaaacggccgggggtgtgcttaatggctattggggctatcaggagtttctggatgagttcccagaacagcgcaatcttacgaacgccctgtctgaagcggttcgggcgcagcctgtgcctctgtctaagccgactcaacgtcctatcaagatttcggtcgtataccctgggcagcaagtttccgattactgggtgcgtaacatcgcaagctttgaaaaacgtctgtataaactgaacattaattaccaactgaaccaagtgttcacccgacccaatgccgatattaaacaacagtcactcagtctgatggaggccctgaaatccaaatcggactacctgatctttaccttggatacgactcgccaccgtaaatttgtggagcacgtactcgatagtacaaatacgaaactgattttacagaatataacgacgccggtacgggaatgggacaaacaccagccgttcctgtatgtgggttttgaccatgctgaaggttcccgagaactggcaaccgaattcggtaaatttttcccgaaacatacgtattactctgtgctctatttctcggaaggctacatcagcgatgtacgtggagatacctttatccatcaggtgaatcgggataataactttgagcttcagtccgcttactacaccaaggcgaccaagcaatccggttatgatgctgccaaggcgagcttggcaaagcaccctgacgttgactttatttatgcctgctcgacggacgtagctctgggtgcagtagatgcactggcagagctcgggagagaggatattatgatcaacggttggggaggggggtccgcggaattagacgcgatacagaaaggtgatctggatattaccgtcatgcgtatgaacgatgacacgggtattgccatggcagaagcgattaaatgggatctggaagacaaaccagtaccgaccgtatatagtggcgactttgaaatcgtaaccaaagcggattccccggaacgaatcgaagcgctgaagaagcgtgcttttcgctattccgataacggtggggcggctgccatggcaagccttccggctacccatgagcttcatattttcggcagtattaatggcgtagactttgacatggtgggtcagggaaccggaaatcccaatgatggctatgaagagttgaacctgaaatctaccaagggcgacctccagttttcaccttggattttggtgccacacatcggttatggatttcatcagtatttaccttatccggatggaatgagtccgttccaggcggccatggttgatggttccggttatcaggtccatcgcacgatgcagttcgaggacggggcctctctgacggtgaattatcgttatacctatgaaggttcacatataaaaggtgaagctcaggtaaaaggcacgggttttccggcggatggcccggtgatgaccaactccctcacggcggcggactggtgccggagcaaaaagacgtatccgaatgataaaaccatcattagcacctttaaatggtcatacacgactggcaacggtaaacgctacagatccaccgctcgcaccacatatacttttgctaaacctatggccgcaaactatttgaaaaaccagcctatgtacgttttccgtaaaaccgaactgaagcacagtaaaactgaactgaatttcaaagaatggcaaaaagcatttacggatgttatgggtatggatgagctgtataagcatcaccatcatcatcattaatagctcgag

SEQ ID NO: 2 - protein, 823 aa, Artificial Sequence
  TqLPNGh biosensor protein
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKTAGGVLNGYWGYQEFLDEFPEQRNLTNALSEAVRAQPVPLSKPTQRPIKISVVYPGQQVSDYWVRNIASFEKRLYKLNINYQLNQVFTRPNADIKQQSLSLMEALKSKSDYLIFTLDTTRHRKFVEHVLDSTNTKLILQNITTPVREWDKHQPFLYVGFDHAEGSRELATEFGKFFPKHTYYSVLYFSEGYISDVRGDTFIHQVNRDNNFELQSAYYTKATKQSGYDAAKASLAKHPDVDFIYACSTDVALGAVDALAELGREDIMINGWGGGSAELDAIQKGDLDITVMRMNDDTGIAMAEAIKWDLEDKPVPTVYSGDFEIVTKADSPERIEALKKRAFRYSDNGGAAAMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKHHHHHH

SEQ ID NO: 3 - protein, 817 aa, Artificial Sequence
  TqLPNGh biosensor protein lacking H6 sequence at C-terminal end
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKTAGGVLNGYWGYQEFLDEFPEQRNLTNALSEAVRAQPVPLSKPTQRPIKISVVYPGQQVSDYWVRNIASFEKRLYKLNINYQLNQVFTRPNADIKQQSLSLMEALKSKSDYLIFTLDTTRHRKFVEHVLDSTNTKLILQNITTPVREWDKHQPFLYVGFDHAEGSRELATEFGKFFPKHTYYSVLYFSEGYISDVRGDTFIHQVNRDNNFELQSAYYTKATKQSGYDAAKASLAKHPDVDFIYACSTDVALGAVDALAELGREDIMINGWGGGSAELDAIQKGDLDITVMRMNDDTGIAMAEAIKWDLEDKPVPTVYSGDFEIVTKADSPERIEALKKRAFRYSDNGGAAAMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 4 - DNA, 1043 bp, Vibrio harveyi
atggtatcaaagggtgtgcttaatggctattggggctatcaggagtttctggatgagttcccagaacagcgcaatcttacgaacgccctgtctgaagcggttcgggcgcagcctgtgcctctgtctaagccgactcaacgtcctatcaagatttcggtcgtataccctgggcagcaagtttccgattactgggtgcgtaacatcgcaagctttgaaaaacgtctgtataaactgaacattaattaccaactgaaccaagtgttcacccgacccaatgccgatattaaacaacagtcactcagtctgatggaggccctgaaatccaaatcggactacctgatctttaccttggatacgactcgccaccgtaaatttgtggagcacgtactcgatagtacaaatacgaaactgattttacagaatataacgacgccggtacgggaatgggacaaacaccagccgttcctgtatgtgggttttgaccatgctgaaggttcccgagaactggcaaccgaattcggtaaatttttcccgaaacatacgtattactctgtgctctatttctcggaaggctacatcagcgatgtacgtggagatacctttatccatcaggtgaatcgggataataactttgagcttcagtccgcttactacaccaaggcgaccaagcaatccggttatgatgctgccaaggcgagcttggcaaagcaccctgacgttgactttatttatgcctgctcgacggacgtagctctgggtgcagtagatgcactggcagagctcgggagagaggatattatgatcaacggttggggaggggggtccgcggaattagacgcgatacagaaaggtgatctggatattaccgtcatgcgtatgaacgatgacacgggtattgccatggcagaagcgattaaatgggatctggaagacaaaccagtaccgaccgtatatagtggcgactttgaaatcgtaaccaaagcggattccccggaacgaatcgaagcgctgaagaagcgtgcttttcgctattccgataacgg

SEQ ID NO: 5 - protein, 348 aa, Vibrio harveyi
MVSKGVLNGYWGYQEFLDEFPEQRNLTNALSEAVRAQPVPLSKPTQRPIKISVVYPGQQVSDYWVRNIASFEKRLYKLNINYQLNQVFTRPNADIKQQSLSLMEALKSKSDYLIFTLDTTRHRKFVEHVLDSTNTKLILQNITTPVREWDKHQPFLYVGFDHAEGSRELATEFGKFFPKHTYYSVLYFSEGYISDVRGDTFIHQVNRDNNFELQSAYYTKATKQSGYDAAKASLAKHPDVDFIYACSTDVALGAVDALAELGREDIMINGWGGGSAELDAIQKGDLDITVMRMNDDTGIAMAEAIKWDLEDKPVPTVYSGDFEIVTKADSPERIEALKKRAFRYSDNG

SEQ ID NO: 6 - DNA, 699 bp, Branchiostoma lanceolatum
atggcaagccttccggctacccatgagcttcatattttcggcagtattaatggcgtagactttgacatggtgggtcagggaaccggaaatcccaatgatggctatgaagagttgaacctgaaatctaccaagggcgacctccagttttcaccttggattttggtgccacacatcggttatggatttcatcagtatttaccttatccggatggaatgagtccgttccaggcggccatggttgatggttccggttatcaggtccatcgcacgatgcagttcgaggacggggcctctctgacggtgaattatcgttatacctatgaaggttcacatataaaaggtgaagctcaggtaaaaggcacgggttttccggcggatggcccggtgatgaccaactccctcacggcggcggactggtgccggagcaaaaagacgtatccgaatgataaaaccatcattagcacctttaaatggtcatacacgactggcaacggtaaacgctacagatccaccgctcgcaccacatatacttttgctaaacctatggccgcaaactatttgaaaaaccagcctatgtacgttttccgtaaaaccgaactgaagcacagtaaaactgaactgaatttcaaagaatggcaaaaagcatttacggatgttatgggtatggatgagctgtataagcatcaccatcatcatcat

SEQ ID NO: 7 - protein, 242 aa, Branchiostoma lanceolatum
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKHHHHHH

SEQ ID NO: 8 - DNA, 741 bp, Aequorea victroria
atggtatcaaagggtgaagaacttttcaccggtgttgtgccaattttggtcgagttagacggtgatgtgaatggccacaaattctctgtctccggcgaaggtgagggtgatgcaacctatggcaaactgacgctgaaatttatttgcaccaccgggaagcttcccgttccttggcctactttggttaccaccttgtcttggggtgtgcagtgcttcgcacgttatcctgaccacatgaaacagcacgatttttttaaaagtgcaatgccggaaggctatgtccaggaacgtactattttttttaaggatgatggcaactataaaacccgtgccgaggtcaaatttgaaggcgacaccttggttaaccgaattgaactgaaaggtattgattttaaagaagatggtaatattctgggtcacaaattggagtataactatttcagcgacaacgtttatattaccgcagataaacaaaaaaacggtatcaaagccaactttaaaattagacacaatatcgaagatggcggtgtccaattagccgaccactatcagcagaacacgccgatcggtgatggtcctgtgctgctgccggataatcattacctttcaacacagagcaaactgagcaaagacccgaacgagaaacgtgaccacatggtactgctggaatttgtgacagctgcgggcattacgcttgggatggacgagctatataaacatcaccatcatcatcattaatag

SEQ ID NO: 9 - protein, 245 aa, Aequorea victroria
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKHHHHHH

SEQ ID NO: 10 - DNA, 7842 bp, Artificial Sequence
  Plasmid - pET-21a(+)-TqLPNGh
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggtatcaaagggtgaagaacttttcaccggtgttgtgccaattttggtcgagttagacggtgatgtgaatggccacaaattctctgtctccggcgaaggtgagggtgatgcaacctatggcaaactgacgctgaaatttatttgcaccaccgggaagcttcccgttccttggcctactttggttaccaccttgtcttggggtgtgcagtgcttcgcacgttatcctgaccacatgaaacagcacgatttttttaaaagtgcaatgccggaaggctatgtccaggaacgtactattttttttaaggatgatggcaactataaaacccgtgccgaggtcaaatttgaaggcgacaccttggttaaccgaattgaactgaaaggtattgattttaaagaagatggtaatattctgggtcacaaattggagtataactatttcagcgacaacgtttatattaccgcagataaacaaaaaaacggtatcaaagccaactttaaaattagacacaatatcgaagatggcggtgtccaattagccgaccactatcagcagaacacgccgatcggtgatggtcctgtgctgctgccggataatcattacctttcaacacagagcaaactgagcaaagacccgaacgagaaacgtgaccacatggtactgctggaatttgtgacagctgcgggcattacgcttgggatggacgagctatataaaacggccgggggtgtgcttaatggctattggggctatcaggagtttctggatgagttcccagaacagcgcaatcttacgaacgccctgtctgaagcggttcgggcgcagcctgtgcctctgtctaagccgactcaacgtcctatcaagatttcggtcgtataccctgggcagcaagtttccgattactgggtgcgtaacatcgcaagctttgaaaaacgtctgtataaactgaacattaattaccaactgaaccaagtgttcacccgacccaatgccgatattaaacaacagtcactcagtctgatggaggccctgaaatccaaatcggactacctgatctttaccttggatacgactcgccaccgtaaatttgtggagcacgtactcgatagtacaaatacgaaactgattttacagaatataacgacgccggtacgggaatgggacaaacaccagccgttcctgtatgtgggttttgaccatgctgaaggttcccgagaactggcaaccgaattcggtaaatttttcccgaaacatacgtattactctgtgctctatttctcggaaggctacatcagcgatgtacgtggagatacctttatccatcaggtgaatcgggataataactttgagcttcagtccgcttactacaccaaggcgaccaagcaatccggttatgatgctgccaaggcgagcttggcaaagcaccctgacgttgactttatttatgcctgctcgacggacgtagctctgggtgcagtagatgcactggcagagctcgggagagaggatattatgatcaacggttggggaggggggtccgcggaattagacgcgatacagaaaggtgatctggatattaccgtcatgcgtatgaacgatgacacgggtattgccatggcagaagcgattaaatgggatctggaagacaaaccagtaccgaccgtatatagtggcgactttgaaatcgtaaccaaagcggattccccggaacgaatcgaagcgctgaagaagcgtgcttttcgctattccgataacggtggggcggctgccatggcaagccttccggctacccatgagcttcatattttcggcagtattaatggcgtagactttgacatggtgggtcagggaaccggaaatcccaatgatggctatgaagagttgaacctgaaatctaccaagggcgacctccagttttcaccttggattttggtgccacacatcggttatggatttcatcagtatttaccttatccggatggaatgagtccgttccaggcggccatggttgatggttccggttatcaggtccatcgcacgatgcagttcgaggacggggcctctctgacggtgaattatcgttatacctatgaaggttcacatataaaaggtgaagctcaggtaaaaggcacgggttttccggcggatggcccggtgatgaccaactccctcacggcggcggactggtgccggagcaaaaagacgtatccgaatgataaaaccatcattagcacctttaaatggtcatacacgactggcaacggtaaacgctacagatccaccgctcgcaccacatatacttttgctaaacctatggccgcaaactatttgaaaaaccagcctatgtacgttttccgtaaaaccgaactgaagcacagtaaaactgaactgaatttcaaagaatggcaaaaagcatttacggatgttatgggtatggatgagctgtataagcatcaccatcatcatcattaatagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 7860 bp, Artificial Sequence
  Plasmid - pET21-hTqLPNG
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgggcagcagccatcatcatcatcatcacggaagcagtggccgtaaaggcgaggagctgttcacaggcgtcgtgccgatcctggtagaactcgatggggatgtcaatggacataagtttagcgtctcgggagaaggggagggtgacgccacgtatgggaagcttacgctgaaatttatttgcaccacgggcaaattaccggtcccgtggcctacccttgtaaccactctctcttggggagtgcagtgcttcgcgaggtacccagatcacatgaaacaacatgattttttcaaatcggcgatgccggaaggctatgtgcaagagcgcacaatcttttttaaagatgacggtaattacaagacccgagccgaagtaaaatttgagggagatacactggtgaaccgtattgaactgaaaggtattgactttaaagaggacggcaatatcctgggccataagctggaatacaattatttttctgataacgtatatattacggccgacaaacagaagaacggtatcaaagcgaattttaaaattcgccacaatatcgaagacgggggtgttcagttggccgatcattatcaacagaacactccgattggggatggccccgtactgctgccagataatcattacctctctactcagtcaaagctgagcaaagatccaaacgagaaacgtgatcacatggttttactggagtttgtcacggccgccggtattaccctgggaatggatgaactgtataagaccgcagggggcgtgctgaacggctattggggctatcaggaattcctggacgaatttccggaacagcgaaatttaaccaacgcactgagcgaagcagtacgcgcccaaccggtgcctctgtctaaacccacccaacgcccgattaagatttctgttgtctacccggggcagcaggtgtctgattactgggtccgcaacattgcctcctttgagaagaggctgtataagcttaatatcaattaccagctgaaccaagtgttcactcgccctaatgcggatatcaaacagcaatcgctttcgctgatggaagctctgaaaagtaaatctgactatctcatctttaccctggacacgacccgtcacagaaagttcgtcgaacacgtccttgattcaacaaacacaaaactaattcttcagaacatcactacgccagtgcgtgaatgggataaacatcagccgtttctttatgtgggctttgaccatgccgaaggcagccgtgagttagcaacggagtttgggaagtttttcccgaaacatacatactactctgttctgtattttagcgagggatatatctcggacgtgcgcggtgatacctttattcatcaggtaaaccgcgataacaactttgaactgcaaagcgcgtattatacgaaagctaccaaacaaagcggctatgacgcagcgaaagcgtccctggcgaagcatccggatgtcgattttatttacgcatgcagtaccgacgtagcattaggggcggttgacgcactggctgagttaggtcgcgaggatattatgattaatggttggggtgggggtagtgccgaattggacgctattcagaaaggtgacctggatattacagtaatgcgtatgaacgatgatactggcattgccatggcagaggcaatcaaatgggatctggaagataaacctgttcccacggtatattccggtgattttgaaattgtcactaaagcagacagtccggaacgtatagaagcactcaaaaagcgtgctttccgttactcagataatgggggcgctgccgccatggcctctctgccagccacccatgaactgcacatttttggttcgattaacggtgttgacttcgacatggtagggcagggtaccggcaatccgaatgacggctatgaagaattgaacctgaaatcaacgaaaggcgatttgcagttttctccgtggatcctggtgccgcacattggctacggatttcaccagtatcttccgtaccccgatgggatgtcaccatttcaggcggctatggtggatggttccggctatcaggtacatcgcacgatgcagtttgaagacggcgcgagcctgacggttaattatcgctacacgtatgagggtagccacatcaaaggcgaagcgcaagtaaaaggtaccggttttccggccgatggaccggttatgacgaacagtctaacggcagccgactggtgtcggtcgaagaaaacgtatccgaatgataagaccattatttcgacgttcaagtggtcctatacgaccggaaacggtaagcgctatcggtctaccgcacgcacgacatacactttcgctaaaccgatggctgccaactaccttaaaaatcagccgatgtatgtattccgaaaaacagaattaaaacattctaaaaccgaacttaattttaaagaatggcaaaaagcctttacagatgtgatgggcatggacgaattgtacaaataatagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat